SEQUENCE LISTING (368 sequences)

SEQ ID NO: 1 - DNA, 401 bp, Homo sapiens
ttcttgtggtgctcgtctttttcttgtttgatgttgctagttgaacacatctcaggttgtgagaagtgcatatttagatgggaacatcttggagagatcttgattggtagagagtaactttatatcagtgagtataatgcttatcagtgaaattctaactcatttaattattacttaattttctgattatgtttttgtatytgagagaagattatttccattatggcaaagtatggatggatggacattgctattgccatttcttgacttagcacacctaggaaggcattttgaaacatactttttaaaaaaagaattgcatatctgtgcacctacaacagtgggtgtgctgaaattctgacttgggttttgatgaaagaattccccaattcagttaaatt

SEQ ID NO: 2 - DNA, 401 bp, Homo sapiens
gcttttcttatgcttccagaatgacttactcagcaatcacttctactttgtattaaaacagttttggttattcaggtaggaagaagagaaaaaaaccaggctgtttccagttgtctggggtttacattatgttacctctcagactgttagtgaaatcagggagactagtggtttttacggcatcagagataccaaatgtayaacagacagatgtcatcttactctttttaagtcatggacaaaaagacagacacattgccctgtaactttcagatcttctgtaacctttttaaacaacataggagagaagttggtacttgtacccaagggaagagaaagttaatgagtagaaaagacagaatttatttaggaaaacgatggaattaagctctggacaaaac

SEQ ID NO: 3 - DNA, 401 bp, Homo sapiens
gtggcattagcataacatggggacttgttagcagtgcaaatgttggtccccacccccgacttactcagtgagaagctctaggggtggggcctgattttaaacaagccctgtagatgattatgatgtatgtgaaagtgtgagaaccagtattctagaagattttaaacaatttgtgttcctatggcagatagctctctggaraactttgtctcctgattcattcccagactaccgtgtgtgtgtacatgtgagtgttggggtcttaccttataacggcatttagtgattggcaaagctcagatgctgggcctctatggggcagaggcacttggacatttgtacctaaggatgtgggccctggagttacatgtacctgaattcagattcttactttgccaagc

SEQ ID NO: 4 - DNA, 401 bp, Homo sapiens
aatttttgtatttttagtagagacgaggtttcaccacgttggccaggttggtctcaaactcctgacctcaagtgatctgcccgcctcggcctcccgaagtgctaggattacaggtgtgagccactgcgcctgacctgaatctttaaattcttaaaatcagttttagtcaccaaagaagatactctgtagcttaataacttygagttactgtaacggaatcaagtcactggtattgagacatttttatttaaaaattttaaattagaatcttactatgtggaaaaatacggtcattgttggaaaatcattttgcctctttttaacagcattcaggcagtttttttttccctttgaaaatagaccttggtctgtgttgttggaaagcatgtaattttcatttg

SEQ ID NO: 5 - DNA, 401 bp, Homo sapiens
tcacctagtagtgcttaatgataccatgtgaatttttatagtcccagctgcactgaagtgtgtgccctgaaacttcggttggattaagaaaagtagcttggtgtgagggctgaaattggtgaaatgggagaacacagggttgcatctatatatattgcaattaatattgacgagtgggcagaagtccaacattatcctagrgggttggctaatgttctttgtacaccaatgcaagtgagtcttttccctccaggtgtgaatagtttaatttagtaggtcgattagtagaagcaaggggtgttttcttgtattccagtttacattatacactcaacattaactagctgtttaaggtacagtgcattgttgagtagttgtggtacaggtatcctgagacgttg

SEQ ID NO: 6 - DNA, 401 bp, Homo sapiens
gtctgtactgctgatgggctgtgtgactttgggcaagtagcttaacttctctgagttcccctgtctctgttttttcatttgtaaaatggagtggaggggacaatattaacttgcaggatggcttgatgatgagaaatgataaatgtcttagtctatattagatcttcagtaaatggtagttgttttgaccactgttactgyaatgagccaaggtggctataagcccttcagtgtttcagtaaggacaagcttacaggtaaccaccaagatcagggcagaacagctgatttaggtctaaacaggttccatcgtgtgtcttcaaaaaggttttcctttttttcctctggagaaaattcagactggtttaagaaggaaactgagagcctcttccctccctatgt

SEQ ID NO: 7 - DNA, 401 bp, Homo sapiens
gtcttactgttctaagaagtgttagcagaaaaatggctaggcattggagtaaccctgtgatttgacattttggggcatcctttcatggtacgatacacctggccaaaagtctcccagctcagaaattctataactagaaatgctttgaataaatatatactgagaaggtattttggggggaaattttaaaattcttatctsacttggctagagcaactgcttacgacactggacttcttaggggtatcgataatggttgtctttgaatggggagtggatttctcagcttcctgggaacagcaacaaaattcccccaacaaaccccagggtgtctgaagcgcctgctttcttctcaaagagcccgatcgaatactcttctgtgggctcaagagctatcaaca

SEQ ID NO: 8 - DNA, 401 bp, Homo sapiens
tttttttgagacagtctccactccgttgcccaggctggagtccagtggcacgatctcagctcactgcaacctctgcctcccaggttcaagtgattttcattcctcagcctcccaagtagctgggactacaggcttgcaccaccgtgcctggctaatacagcttttttttttttttcttaattttatcataggtaagggaaracgatccaatgtgcagagaaggctcaggttttcattttagtctgcgggtgattgatttctttctttcaaggggctggttgaggaggtcagagtcttagaaagggagaagaaatcagggaaaaggagaaaagaaggaatgagatttatgaccctctggatcccagattttatgtcgcgtaaccattcccaatactggaagt

SEQ ID NO: 9 - DNA, 401 bp, Homo sapiens
tggaactcagagcagtgatctgcatgcaaaggcaaaattaaatagtccagcaactgctttgaccgagagacacacatagtactattctgggtcctgagtgctgcttccctttcatttctcccctaggcacagattccactggctcacagagtgatgagttagaccacccagggccacttatgttatactgagggcggatgstgatagtgcttatagaggaattatttcaacaatgagacttttccattattctttctgcaatcaccctctatactcagaatgtggaaaccttggcatctctcacttaagaacactgaattcatggtgttgctcagagagtcctgtcctggttccataacactgtgataataactgaagatctttaaaaatgtatgtatctt

SEQ ID NO: 10 - DNA, 401 bp, Homo sapiens
cattgtagggctaatactgtattgtaatatttgtttcctggtctgtgtcttttactctgaggagagagagggcagggagttaaatcatttgtctttctggccctggttccaccagagggccttggcaatataagcactgggtttgttcagtaaatgagccccagggtgagagtcaggaaattttcttcctagtcttcatcyagtcattaaaggtgaggagtgaattcaggcagtgaatttaggcaagtccctttactttcctgaaaacaagttttctgatcaatgaaacgagtgtgttgagttcagggctagtaaatctgtttttggtgggttgaatactgccggcacagtgttttcaaacacttgaattggaatgtctttagatggaatctgtgccttct

SEQ ID NO: 11 - DNA, 401 bp, Homo sapiens
ttgaattggaatgtctttagatggaatctgtgccttctagtttgccataatccccactgttccctattatattatgttgtatcagcagcctgcttctatcatttgcctgcagagtctataagcatttatgattccttgtaattattgatcatgtggtcttttgttgctatactaagggtctaaatctgattcaggttagcygttgatgcctttgactgtaactgtaattctctaactttcaacccttttatcatcaaggacctcaactattattttttgttccatatttgaaaacttttggtgttccagacacactgcattggttaataactaattttcccgttgtaaaaacagacacgtgtaactgaacacacaaatgagccatcaacagtatgaatata

SEQ ID NO: 12 - DNA, 401 bp, Homo sapiens
tttgtgcaatgagttggttgacgtttcagctagcttgttcagttgtttcatgggtaagtgtatagtttttgtttgaagttttgatatatgaaaaatagttccgaggtcttctgccttattagacatgtgatagacaaaggattctaggtgagaacttagtttattcttttatgagttgctgaactgccctggtcattagarcatcaagtaagttgtttatccacatgcagttcatgtcttttcagttgtctccttattggtcagcttagagagggctctacaaaacctggcttttacattcagtgagctgaaaagagcttcctatatgagtttacccaaaggcccttgttacattttctccctttagtcaagagtactttggaattttacattttctccct

SEQ ID NO: 13 - DNA, 401 bp, Homo sapiens
tgtatttttaatagagatgggttttgccatgttgcccagactggtattgaactcctgggctcaagccatccacctgcctcaccctccccaagcgttgagactacaggcagtgagccactgcatctggccaaacatacatttaaatagagcagatgaaagatctatagtgtagaagtaatctgcatataaaccagtcttaarttgagacgtgacctatgactgttaaagcagtccacatcacacaaaaataaagggcttggaagatagcagcctggtttaaccaggttagatataatttaacaggaagagatagtgtctacattgttgctaaaagcttaggctctggggttcctaatttgggtttaattctgtcttggttacttgttaattctgtcaccttg

SEQ ID NO: 14 - DNA, 401 bp, Homo sapiens
caaaggatttatcatcatattggttatagaagaaacaatgaatattcatgatcatttttatcaagaatataattgaaatatatttctgagcgttgaggtttttctcttaaagaaaagtgtaaaggtatactttcttaagactgctcatacatgctggatcacccatatacatttccaggaaattgtatgttctagcaattstatgttccaaatgtgctgctttcagttttttcgagaaacaaatgatgtttgactttaaaattctagagttggccgggcgcggtggctcacgcctgtaatcccagcactttgggaggccgaggcgggtggatcatgaggtcaggagatcgagaccatcctggctaacaaggtgaaaccccgtctctactaaaaatacaaaa

SEQ ID NO: 15 - DNA, 401 bp, Homo sapiens
cttttgtggcaaaggcaccagggcagtaatcattattaatggatttcattatttgggagagcattttactgactatagagaattcattgcattgatctattgtgccacagttttattataccgggtagtttaaaatgatattctctaaactgttaggctgaaaaattgtccagtgttggcagttatcccatcagtaagacmttccttttactaccgaaaatacatagttctgtcgaaagagaccttttgactggtccccagtgccctgactgtgtttaaatccctcactagtgaaataggaggattactactgccatcattgttctaaaacaaacttccttttggaagataacagcagcggtaaatcagatgttgaagaaagtaccagcttaacttcactt

SEQ ID NO: 16 - DNA, 401 bp, Homo sapiens
taaaccttgtgtggggctgcctgctggggttggagttcttaatgaacatacaagtgaatacactgaggcaaaaaaattaaagctctccaactgtggggtattcattctgttcactgtggccagtgtggtgatcagtactggccacaccagtggccaaagagaactgcattcatcatgtggctgttctatagctgtgagctrtggtgactgttatttttcctagtgatagttttcagtgacagcatagattctggtatcatatccaaggaataaacaaacactgtttttggctttttgtttttttgtttttgacataaaaataataagctatttttggcatatgcagacttttcacaaagtgattgttttcttgagctctggactacttggtaacattcata

SEQ ID NO: 17 - DNA, 401 bp, Homo sapiens
ggttgcagtgggcctgagatgctgcatttctgacaagctcgctgagacatccctgctgtagaacctctgaccacaccctgagtagcaagggtctgctttaccacttagctaactttgtggctttgggtatactgtttaacaactctgcatctcagtttcctaatctataaaattgggatattaatctttgttctgcctacytcattgggttgttatgagaataaaattagataatgtagatgctttttttttttttttttttgagatggagtcttgctctgttgcccgggctggagtgcagtggctcgatctcggctcattgcaacctctgcctcccgggctcaagcagttctcctgattcagcctcccaagtaattggaattacaggcgcctgccaccat

SEQ ID NO: 18 - DNA, 401 bp, Homo sapiens
gtgtcaaataagcataaggcagcaaggacatttgtttatagatacagacattctttggatacctttcatcaaatcagtgtgggctgttctttaataggaggggcctggaactgtgaggcaggagaagggaactggaatccctgcctccctagtacccattctgtgcctttggccaaatcatctcccctggtcccctgttgygtcatctttaaaataagggcttgacctaggttagtgctcttaaagggtagttgcaagactagcagcatcagcatcactgggaaacttgctagaaatgcaggttagattcctagccccttccgagacctggtgaatcagaaactctgggaatgcagtccagcaatctgatttctaatgagctcttcaggtgattctgatgc

SEQ ID NO: 19 - DNA, 401 bp, Homo sapiens
tctagcttatccagttttggggctgctttagtagcccacaaacgctgactaatgcagatgactgtacacactgagaaagttctcttccattttagtttcctccttactctcttcctgttccccgaatggttgtcatcatgatatagcaccaagtcacattttatagacttttcaataactttctaaagttgatcacaatcwagattatttgcattgttgtgagatgagggaaactgagaaagtaaaatacatgttttgttttgttttgagacagagcctcactctgttgtccaggctggagtgcagtggcacaatctcagctcactgcaacctctgcctcccaggttcaagtgactctcctgcctcagcctcttgagtagcagcgattacaggcactcgcc

SEQ ID NO: 20 - DNA, 401 bp, Homo sapiens
ctttctctctagacatgcgccatgtgcaacacacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacmcccctctataatcctgtatacttaaattcctaaatagttgtttgggtgttcactatattggaacgctttaacttgtgttcttaataatatctttaggaaaagattaaagcatgtttctgcatataataatattagtaacaaatgatggaagattttgctccaaaatgagttaatgtagaaaacaggtagtgattaaagtg

SEQ ID NO: 21 - DNA, 401 bp, Homo sapiens
ttctttggtgacctggtggtgtgctatggaagcgaaattggtgtgcctgttgaaaaagttagtagtcagaaaaaaagacatacttttataagtgagactgtgactattatgctggacacctgcattctaactagcaaaacagaaaatactttgttggttttaatggtgctttgtttttatactgcatggtatctatttttrtatgctggggtcttaaaatgcttggagcatctagagaggtaactaaaggaatgaagtaggccatagacatagcaacagcccatgtcccattgaaaggccatgagtcccagttgaggccccttggctctagccaattggtgctgtgtgaaaatctgggcccagtgttgtcagttcttttaattttttcctaagaatctgga

SEQ ID NO: 22 - DNA, 401 bp, Homo sapiens
ttgtggcttagctggaatatattgatgataagaatggctcagcagacagaagtcttgggactctcaaaaaggctccaagtgtgctttcttttaaaaaagttatttaggcccatcctttataaacacccaagtagatggtctgatggggtcatggtaacaaagattcagcttctatctaggtggatggtaagacccgctaayatcttggcaaaccgtgttattgggccattaaggaccagtgcttgaattctggggctgaaaattcaacgtattcccttataagaaaatgtctgctcatgataagaagtcacacaaagtacaacctcactatagtacaggatttagaatctttatttctccatctcatcttaaacccattggaagttagcatggattagagg

SEQ ID NO: 23 - DNA, 401 bp, Homo sapiens
gtgtgctttcttttaaaaaagttatttaggcccatcctttataaacacccaagtagatggtctgatggggtcatggtaacaaagattcagcttctatctaggtggatggtaagacccgctaacatcttggcaaaccgtgttattgggccattaaggaccagtgcttgaattctggggctgaaaattcaacgtattcccttrtaagaaaatgtctgctcatgataagaagtcacacaaagtacaacctcactatagtacaggatttagaatctttatttctccatctcatcttaaacccattggaagttagcatggattagagggtcggttgactcttctcaatgacggggctggcatataagagctaaaatttttattattgagttactactcaaggtttt

SEQ ID NO: 24 - DNA, 401 bp, Homo sapiens
tataaagcataaatatgtaatttgttcagctgttgtaattaaatatgtatgtgtgaaacagccaccattcaggtcattaatgatgcgccatgccaaattagagcttacagacagtaatgtacattgttgtgcaatgagggaattgcaaataacatggctaagcctttcctagtaaagggatgcattcagcagctttaaaargaatatttacatttgtaacataatttttatttagaaggtacatttttgttcattgtgaaagtctgtaagatggaattactttcatctccactttagttttattattgttttaacattttatcatacaaatgcaacagactttattaaacatgctgcttggtgataagtgttaagtatctacttacatataaaacagcagt

SEQ ID NO: 25 - DNA, 401 bp, Homo sapiens
tgaaacagccaccattcaggtcattaatgatgcgccatgccaaattagagcttacagacagtaatgtacattgttgtgcaatgagggaattgcaaataacatggctaagcctttcctagtaaagggatgcattcagcagctttaaaaagaatatttacatttgtaacataatttttatttagaaggtacatttttgttcaktgtgaaagtctgtaagatggaattactttcatctccactttagttttattattgttttaacattttatcatacaaatgcaacagactttattaaacatgctgcttggtgataagtgttaagtatctacttacatataaaacagcagttacccctggttttctacatggctgtgatagaactgatgtatcatagcactgtg

SEQ ID NO: 26 - DNA, 401 bp, Homo sapiens
cattcaggtcattaatgatgcgccatgccaaattagagcttacagacagtaatgtacattgttgtgcaatgagggaattgcaaataacatggctaagcctttcctagtaaagggatgcattcagcagctttaaaaagaatatttacatttgtaacataatttttatttagaaggtacatttttgttcattgtgaaagtctrtaagatggaattactttcatctccactttagttttattattgttttaacattttatcatacaaatgcaacagactttattaaacatgctgcttggtgataagtgttaagtatctacttacatataaaacagcagttacccctggttttctacatggctgtgatagaactgatgtatcatagcactgtggaatgtcttgat

SEQ ID NO: 27 - DNA, 401 bp, Homo sapiens
atcataacagtttggcttgctttacctaagttattgttccataatatcaaaaaaattacttaaaataagttcttctcttcatattccccgaagtttttgtccagttttctgtatagctttttggttcagccaaaaagagacatttcatttgcagcattagggaaaagtttaattattgtttatgaagatagaaatgttatrtgaatgacagtgatttaaaaatattattacttatgattgtagtcaaccttttccccgaatattgaaaaccatgaaaaggctttgccctgacagctacatgcttagcattaactatacttgcaagttttccaaaaagatttttttcaagacctgttttcatttacttcctttatcctaattagagatcgtaatcttttgat

SEQ ID NO: 28 - DNA, 401 bp, Homo sapiens
ataacagtttggcttgctttacctaagttattgttccataatatcaaaaaaattacttaaaataagttcttctcttcatattccccgaagtttttgtccagttttctgtatagctttttggttcagccaaaaagagacatttcatttgcagcattagggaaaagtttaattattgtttatgaagatagaaatgttatatgratgacagtgatttaaaaatattattacttatgattgtagtcaaccttttccccgaatattgaaaaccatgaaaaggctttgccctgacagctacatgcttagcattaactatacttgcaagttttccaaaaagatttttttcaagacctgttttcatttacttcctttatcctaattagagatcgtaatcttttgatggg

SEQ ID NO: 29 - DNA, 401 bp, Homo sapiens
atgaacacgaaggaaggaactgaaagaaaacagaggagtttaaagttacttctatgaacttttcccagacataacacacagttctctgacttgacttacattcttttaaccctgaaagttccatctctgtgtctgagcagaatgctggactgcttaacgttaatatgagaactaatgtgagatttaaacacttttaaaagrttttaatgtctaaggatagctgcaaattccaaatatgaaaatttggcaggcttttggggggtaacagaaaactttttaaacttacatgctttatctttgcaccctgacatgtgttaagtgagtcaaatcttcctgttaattactcttgtgacattagcaagttatgtaagcccactatacctgtttccacatatgtataa

SEQ ID NO: 30 - DNA, 401 bp, Homo sapiens
accctgaaagttccatctctgtgtctgagcagaatgctggactgcttaacgttaatatgagaactaatgtgagatttaaacacttttaaaaggttttaatgtctaaggatagctgcaaattccaaatatgaaaatttggcaggcttttggggggtaacagaaaactttttaaacttacatgctttatctttgcaccctgayatgtgttaagtgagtcaaatcttcctgttaattactcttgtgacattagcaagttatgtaagcccactatacctgtttccacatatgtataatgaagacgttaaagaagataggtagtagtcttctgagccctaaagaaattgaatttgaataaacaactggaataagtataaaatgcatttctagtttttatgtggaaa

SEQ ID NO: 31 - DNA, 401 bp, Homo sapiens
cttctgtatctacctttctaaccccactttttttttttttttttttttaaccgtgactccttagatgctccagccatactggtttcctttcagttttcagaacctgccatcccatttgctacttgtctgcttagaatgcatttttcccagctccttttgtggctggcttattcttacctttcagttctttcagttcgaatratagccctgtaaaagagtgttctgtctaaaattgtccttcctgtttatattttccataatactaataacagtctgaaatgctcttgttaatttatttgattactcattttatttatttatcttttgcactatgatgtcagtcccacaaggatgcaaactacgtttactaccttcttttctaccttttgcacttttcctaa

SEQ ID NO: 32 - DNA, 401 bp, Homo sapiens
ccttttgtggctggcttattcttacctttcagttctttcagttcgaataatagccctgtaaaagagtgttctgtctaaaattgtccttcctgtttatattttccataatactaataacagtctgaaatgctcttgttaatttatttgattactcattttatttatttatcttttgcactatgatgtcagtcccacaaggaygcaaactacgtttactaccttcttttctaccttttgcacttttcctaatcacattgggaataaaatgttgggcaagttagaatactccaaaatatttcatttaccttaaattttactcaatcctacattttattacctatactcataagaattgtattataaaatacattgttaaacgaatgttttcagtgctccattga

SEQ ID NO: 33 - DNA, 401 bp, Homo sapiens
ataacagtctgaaatgctcttgttaatttatttgattactcattttatttatttatcttttgcactatgatgtcagtcccacaaggatgcaaactacgtttactaccttcttttctaccttttgcacttttcctaatcacattgggaataaaatgttgggcaagttagaatactccaaaatatttcatttaccttaaattwtactcaatcctacattttattacctatactcataagaattgtattataaaatacattgttaaacgaatgttttcagtgctccattgagagtcggtggagcacactggttgggagaagacagagctgtgagccatccgtctgcctgtgcttgagtcttggctctgccattgactagttgtatgaactgccgcaggtggttc

SEQ ID NO: 34 - DNA, 401 bp, Homo sapiens
agtcccacaaggatgcaaactacgtttactaccttcttttctaccttttgcacttttcctaatcacattgggaataaaatgttgggcaagttagaatactccaaaatatttcatttaccttaaattttactcaatcctacattttattacctatactcataagaattgtattataaaatacattgttaaacgaatgttttyagtgctccattgagagtcggtggagcacactggttgggagaagacagagctgtgagccatccgtctgcctgtgcttgagtcttggctctgccattgactagttgtatgaactgccgcaggtggttcagccactcagaacctctgtaaaagtgagatgtaaaaacactttctacatcataggattattgtgaagattaaat

SEQ ID NO: 35 - DNA, 401 bp, Homo sapiens
agtgtcagagaacagtctcagaaagatctgttcctttctttctagactcagtaccacagactggcctatcctctgcaactttgcttagcagcaggagtagagaagtattgattgcccacaacttgcctttaagtcttgtttctgtggtgcaggatttttaaaaagcatttaatgttttccctgccttgaagacttcagaaycgtataaatgccactgtttaaagtcctgtccctgctgaaaaccagggcaggtctcatcacagccccatctccattttccttttgttgaagtgggtctgtgtgagagcgggctgtgccctccttctccacagggtggggaaaaggcagccctgtagtaaggaggttgaatagcctcgctcactttgcctcctgcttgaggt

SEQ ID NO: 36 - DNA, 401 bp, Homo sapiens
ttggccagcctggtagttttcttacatgaccatctttagatttcagagaaggaagaacatgatcccagaaagcacacagagttacaacatagcaatagcccctccgagctcaacaaaaacatctattgtgtcatgggctgcaaagaaagaggtatgctgggaaccaataacaagatgctaggaatttttttttctgttctrttttagcttgaaaaacttgttttccccatatgagttgtgcatttactcttggatcttaaaagaggacaatttattaatctgggtttaaatcctggcccagccacttggatgctgtttgattttaggcaaattatttgactttgtgctattgtgtcgtcatcgacatcatcatcatcgtcatcttttagctgaattctgtg

SEQ ID NO: 37 - DNA, 401 bp, Homo sapiens
taatttgccattaaaaattgaactgcagggctggccttgcttgacttccaatttgggcaggtgttagattgtgacttacataacattgcaaactcatccattgccctgatattccagaaggataggtgccaatggtaacttaagattgagatttcacctttatggcttgcttcctatttctggtaagagggaattaaagcrttatctccctatagtgtaattatggtcataaacttgattagagttctttaaataaaatccaactaggaaaaaggcaggaaaacactgtggtcaattttggggaaggaggatggaatacaggcatgaccttcaccttggtcaaaatatgttccctaagactctgggaagtcttgtatatataagattcatatggcagggtt

SEQ ID NO: 38 - DNA, 401 bp, Homo sapiens
ggtgtggaagtcctttctgtttgttagttttccttctaacagacaggaccctcagctgcaggtctgttggaataccctgccgtgtgaggtgtcagtgtgcccctgctcggggggtgcctcccagttagactgctcgggggtcaggggtcagggacccccttgaggaggcagtctgcccattctcagatctccagctgcatsctgggagaaccactgctctcttcaaagctgtcagacagggacatttaagtctgcagaagttactgctgtctttttgtttgtctgtgccctgcccccagagatggagcctacagaggcaggcaggcctccttgagctgtggtgggctccacccagtttgagcttcccggctgctttgtttacctaagcaagcctgggcaat

SEQ ID NO: 39 - DNA, 401 bp, Homo sapiens
cccactttttgatggggttgttttttcttgtaaatttcagttccttgtagattctggatattaggcctttgtcagatggatagactggaaaacttttctcccattctgtaggttgcctgttcactctgacgatagtttcttttgctgtgcagaagctctttcttttaattagatcccatttgttatttttggcttttgttrccattgcttttggtgtttcagttgtgaagtctttgcccatgcctatgtcctgaatggtattgcctaggttttcttctagggtttctatggttttaggtcttatgtttaagtctttaatccatcttgagttagtttttctataaggtgtaaagaaggggtccagtttcagttttctgcatatagatagccagttttcccaa

SEQ ID NO: 40 - DNA, 401 bp, Homo sapiens
ctgttatattgaggaatagactttggagggagaaaacaggggcagaggaagggaggttagttaggactattgtaatagcccaggaaagtgactgtgaaagtggtaagaagtgagagtattctggataaatttttaaggtaaagctaatgtcatttgctaacagattgaacatggtacaaaagaaaaagaggagtcaaggaygacctcaaaattttgtctgagtaaccagagaatagaattgatattcattgaagtggggaagactgtgggagaaacaagtatggtaaaggtgagggagaaattaagagttaggttttagacatacatattaagtgtgagtttcttcttagacaccctggtggacatgtcaggtaggcagctggatttataagtgtcaaatt

SEQ ID NO: 41 - DNA, 401 bp, Homo sapiens
ttctggtggagagggaccccctgcagctcagagacatggttcctgtgcctggcagcttagcataaacccctcccatgaagttctgagttacacagagtttccagatctgtgaactgcattaatgtctcctgctttagcttaagttccgtacatggctggcttccttaatagactttattgttgtttaaaaacatctttggyaatagagatcattgatggactttatattagataaagatgattctaggcatctattataatatgtatcgcatgttcatatatattggatgcatccctactggagcagcacaatatactttagacctaagattctaatagcttcacatatgaaggacaacttggttctgcaccaagtacaaccgatgtagacactaaccttg

SEQ ID NO: 42 - DNA, 401 bp, Homo sapiens
gcatttttaacctattcatttgatcttcactagggtgttattcctattaaaaaatctacattgtgtatatgtatacagttttttccttgccaaatggaaaaaaattctaaccaattatgatttgtaaggcctttagagaaacatgaaatattttcataatgttactttcatttttttcccatgcttaaaataacaagatawgaatatttgggagaaagcaggccagagagattaaaggatatgcctaaagttgtatgctatttggtaaacagaatgagaccagaacttcaggaaagtcattctacatcagtacaaatattggaggtggacattcttcttcagtggaaaagcagcctggaggaaccaaatagtcttttaaaaaaacactttttcaagtgcag

SEQ ID NO: 43 - DNA, 401 bp, Homo sapiens
aattctgataaggtttctgtgagtttagtgatacattctttaaatgtaatttgaagcttttaaaatactgtaatggcagctcttgacctttaaaaatcacatggagcaagaggtggtgaaaaatgtttatttactctgcattagtggctaacctcctgttccttcctagaaccagtagttcttttatactcgggaactcaygcatgtcaccctcattcttccaacacgaaccagacaggcccacttgagctggagaaacaatggcctgtagccggtcctatgcctcagtgcgaaacaacaatgcaggcctcttgacgaattctggcgtcgcgagtggaatcttgcagttcaacatgtctgctgaacagaagttggaaaaagaaaagatcatcttgtaaaca

SEQ ID NO: 44 - DNA, 401 bp, Homo sapiens
aacaatttatatttctgatatgctttgatagttacatgggaggacaaaaatgcatagtgacaaatgacgataattcaatgcagtggttgcaatatgtcatattattttcttcaattaatatttctccctacatgccatatttttcctttgacctaaaagaatgtaaaaaaggggctttaccataattctaaactaacaaastttctgaggaggggaagagtaaattatggccactcaaattttaaatttatgattatatcaagaatccttctacttctcaccccctccacactgtggcctttctggtccagaccacttccacctctccctggattactgcaactcccaaactggtctccctgtttctgccatgcttccgtagagtgtattttcaacacagg

SEQ ID NO: 45 - DNA, 401 bp, Homo sapiens
tccaagccagagatcacagtggcttcaactctagtagtagcagtagagataagacagaagtgggcagatttgagagatatttatttagaaaacagaatcaacagacatggtgactaaatggagagggagactcagtctctgcacccagtgaggacttgggtttgagcagggtgcagtagtgacacatgattgtagtcccamtgacacaggaggttgaagcaggagcattacttgagcctaggagttcaagtacaacctgggcaagactggactatctcttttttcttttttttttagagttgtcacttagtatcttaggcatgtttgcttgggctctttaaacttcaatttatttatctgtacagtgataacaccaccaccatctcaaaagggtactatga

SEQ ID NO: 46 - DNA, 401 bp, Homo sapiens
aatcttctcttttctatttcatattcttttttttttttccttttgatcagaagtgattccttcctagataattccacatggttctgtttttcctattgtcttgacattgtgtaagaatttgaaaaagaatgttgcaatctgtttctctgaactgtggtttatcaagaggatctgttttaagccaaagagagtttccttgtrtgggatcatgctaggaggttgtgcgcgccttgcaggcaaatggaggactgcagcctcatggaattgtgctgctgtgggggctgtaggctcctggccctcttggagggtggatactccatgccttgtatcatatagcttttagctgccagcatctgctaggctttggaatacccaactctgattttctgggaggcttttat

SEQ ID NO: 47 - DNA, 401 bp, Homo sapiens
cttgcaggctaggtgcagtggttcatgcctataatcccagcactttgggaggccaaggcaggcagatcacttgaggtcaggagttcgagaccagcctggccaacatggggaaccccatctttactaaaaatacaaaaaatgagccaggtgtggtggcacacacctgtagtcccagctacttgggagactgaggcacgagamtcacttgagcccgggggggcagaggttgcagtgagctgagattgcaccactttattctagcctgggtgacagagcaagaccctgtttccaaaacaaaacaaacaaacaacaacaacaacaaaaagactggcaaaatgaccactagctaggtcagtctcagtggtcccagagtgagctagtcaggaccccatttcttatct

SEQ ID NO: 48 - DNA, 401 bp, Homo sapiens
aggactcctacattctcacagccaggagcatccaaaagctgatggggcctcatcccagttctggctgcaggacacttccctcacaagagcatcggctatttcagtttattttcatctaactataaagggatatagaaacagcataataaaaaatgcttgcgctgtagaatttacaatctagctagtgatgtggaatagagmacagatgacaatctatggtcatgtaaagttaatgagaacagtacaggcagtctagtccatacctgcaaagtacaatgaagtcaatcaaggaaggtttcttgaaggcatgaattttgggtgctgctttagagggtgattcttatgggagcaggggattccttgggggtcaaaatggacattgccaaggtcaaatgagggga

SEQ ID NO: 49 - DNA, 401 bp, Homo sapiens
ttttgatttgcatttctctgatggccaatgatgatgagcattttttcatgtgttttttggcacataaatgtcttcttttgagaagtgtctgttcatatccttcacccactttttgatggggttgtttgtttttttcttgtaaatttgtttgagttcattgtagattctggatattagccctttgtcagatgagtagattgygaaaattttctcccattttgtaggttgcctgttcactctgatggtagtttcttttgctgtgcagaagctctttagtttaattagatcccatttgtcaattttggctttaaaacagataatttttaacagtagttatttctaggtggtgaaatataggggctttattctaaaaatgtatttatatattatatatttgtgta

SEQ ID NO: 50 - DNA, 401 bp, Homo sapiens
ctttctccaatgaacagatttcttcatcgctagcacaggagcaaaatgtgtaattattggccatcattggggcgatttttcagtgattcttttgtgcatcgtgaataaagcataatgacatattttattagagggcaaatagctgtttgttaatggaaatgatttgaaatccaaaaaactagaagctaggagcatgttttyggtatcagcgtgaattcaaatgtattttcttttaactctttataaaaggagcatagaaatcaaatctaaactgagcataatatatttaatgatatgtacaaacacagcattgattaattacctcaaatgctttcagtagtacaccatttattttgtttcagtcatgtgttaaattttagtatttcatatttaaatcctta

SEQ ID NO: 51 - DNA, 401 bp, Homo sapiens
tttgccagacttgcctcactggggatatcagcttttccagccctgccattacatgtctgtgccaccctcttaagtttgtacttaatattatgtctcctcttgctgcgactttccatcatttgtacatgttttcaagtctgggctcaccagggtttccttgtgaactcccctttattctgtgtcagggtctgaacaactgcrttttttaaagttctcaacattcttaagaagcctccctttttttttctttttttttattattattatactttaagttttagggtacatgtgcacaatgtgcaggttagttacatacgtatacatgtgccatgctggtgtgctgcacccattaactcgtcatttagcattaggtatatctcctagtgctatccctcccccct

SEQ ID NO: 52 - DNA, 401 bp, Homo sapiens
cacatggttcgcattttccaccctcccccgcctctcgcgccgaggcagcctcagcccggcttgctcacttggagagtgcggccggggctggacttggggcgcagcccgggaggcccgagcctgcttggggctgccggctgcagactccgctgtgggcagagcagcttgcttggggatcactacggccgggagaagtctggscgggaggagtccagcacgccttggaaattgaagtattctccgattctggtaatcaggccaaatttgctcaggcaggaagttcaaatgtcacctaattggtttcgttcttatgcttcacttcattttcctcggaaatggaggtcccgaagttactactagtaacttgcatgtaactcattcccagacgaagtcatattcac

SEQ ID NO: 53 - DNA, 401 bp, Homo sapiens
caaaatataaaagaataaaattaattaagttggcactggacttccggtggtcagtcatgtgtgtcatctgtcacgtttttcgggctctggtggaaatggatctgtctgtcttctctcataggtggtattcacagccaacgactccggcccccgccgctacaccattgccgccctgctgagcccctactcctattccaccayggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattt

SEQ ID NO: 54 - DNA, 401 bp, Homo sapiens
attctgtgtcacgctctgtgtaagtgactccatgatcaaagctttcctgttgtaattgtgtggatttacttgttgcccactgtccccataccctccacccccacatggtgtgctttctccagaaagggactacttctgctgaccacacagaagacgtgtgtaaagtctgtgtatcaatgaatggattctcatctttcatagtttttttttaaatagttttatgtgtgtttaacttaatttcacttaaaaagatatttaccagaagctgaaagtagggtgtgatgaggttgggttcaggaaggactggtatcacatggcttccctaagttgtatattacattgttaggacacctgacagagctgtggattagtgaatcttacggatggctcttttcagttga

SEQ ID NO: 55 - DNA, 410 bp, Homo sapiens
cacttgaattggaatgtctttagatggaatctgtgccttctagtttgccataatccccactgttccctattatattatgttgtatcagcagcctgcttctatcatttgcctgcagagtctataagcatttatgattccttgtaattattgatcatgtggtcttttgttgctatactaagggtctaaatctgattcaggttagccgttgatgcctttgactgtaactgtaattctctaactttcaacccttttatcatcaaggacctcaactattattttttgttccatatttgaaaacttttggtgttccagacacactgcattggttaataactaattttcccgttgtaaaaacagacacgtgtaactgaacacacaaatgagccatcaacagtatgaatataaaagtg

SEQ ID NO: 56 - DNA, 401 bp, Homo sapiens
ggttctttcccctctcaacccaggcttccttagtcacgtgactggaatttaattatcagtgccataaataatcttgtgaatggaagcagtgtatttggcagtgaatttctgcttcctaaagagaaaggaacctttagaagttatttgaaataattctgtattagccacgatcctggaggcaaatggtcacagaagcagagsatggtatccccagagaaaagtgggttttagatgagtcagataatgtggatatgtgctggtgacgaatgacatgaaggttggatgtattttttaaaatacaaatttaaagcaggctgtatttagaagtttatttataattggttttaggataaagccagcctgttgatgcataacagagttgatcttttggttccattagc

SEQ ID NO: 57 - DNA, 401 bp, Homo sapiens
gatcaaatacttgacataatattcatgttagaatgtagctacataggaataagttgtaggatctgagacactttatggaatgtttctcaaaaacaatcaaataattttggtcttttctttgtgaagcaatataacctgtggttacgagttatgtgtcttagaaagacttagatttgaattctctgtctgctgcttgttagytgtgccattttggatcagttcttttctttgagactcactttattttaaagcaaagttaatatacctacctaattggttttttttgtaaaggattaaacatcatggtatttgtaaagcattcagcactttgcctagaattttatagttccagtaaatagtagctatttttatcattattgtcaccatgatattgtttttag

SEQ ID NO: 58 - DNA, 413 bp, Homo sapiens
ctagacacaagtctgatttttcattccagagcagcaaataaagtcatagtggacagctgcttcagtctggaaactagaaacaaacaagaggtgttagctggcagctgaacaatgaagaaagacatggagacactgtccaagaggtcgagatggatagtagcttgagatcctctctttctctctagacatgcgccatgtgcaacacacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacccctctataatcctgtatacttaaattcctaaatagtt

SEQ ID NO: 59 - DNA, 405 bp, Homo sapiens
aaccattctctttcttttctttttttcaaaattagagacagggtcttaatttgtcacccaggctggagtgcaatggcacgatcctagctcactacagcctcgaactcctgggcttaagggatcctcctgccccagccgcatgagtagcaagtgcatgccaccatgcctggttaatttctttcttttctttttctttctttctttttttttttttttttggatgagatatgggtctaattatgttgaccaggctggtctcgaactcctggcctcaagcagtcttctcaccctaggcccccagaatgctgggattacaggctttagcaaccacacccagcctgaaccatttcctttctgatttaacttaggaaagtttgctgcatagtaggagctcagctaacattt

SEQ ID NO: 60 - DNA, 404 bp, Homo sapiens
tctttctctctagacatgcgccatgtgcaacacacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacccctctataatcctgtatacttaaattcctaaatagttgtttgggtgttcactatattggaacgctttaacttgtgttcttaataatatctttaggaaaagattaaagcatgtttctgcatataataatattagtaacaaatgatggaagattttgctccaaaatgagttaatgtagaaaacaggtagtgattaaagtggt

SEQ ID NO: 61 - DNA, 404 bp, Homo sapiens; identical to SEQ ID NO: 60

SEQ ID NO: 62 - DNA, 401 bp, Homo sapiens
tcaaagcatcataggatgttatagttataagggaccataggtttcatttaacgcacattttagcatccaggtcctacataaaatgtatattttgtgattgtgaatgggagttttccatatttaaaagttaagtttattaatgtgtcattttacccatcatgtatttgtggccatttttttaataccacgaatgggaacatmctgtatcatagtttctttttatttgtctggatgcttgtgtatgccttttagtctactaatatgctcacatgtgcaaactaacaagagaagatggcaatccggtattataaactggtaaagagttatagtgcagtaagactggattaactgtgctttcagcagtcaagttgctgtaagagttatattgtaaagttttagac

SEQ ID NO: 63 - DNA, 401 bp, Homo sapiens
caggttttgaacacttttgtgaagctgcttttgtaaattgtttttaatattttgtttttaactctaatttctagtatagaaatacagttactttgtgtatagtgatcttatattcagcgatatttacatatttttagccaataatgtattggtaagttcttttttgtttactatttcgataatcatatcaaatgcaaacartgagaaagatgttcatttctattctatgcttttatcttccttacccttctcattgcactgagatctgcaactcagtgttaaattaacctgtgatagtgggcattcttgatttttttttcttgatttcctatttatttttaatccctgaatgaatcagagttaatgccttaatatttaaatattactttcgctgctcacat

SEQ ID NO: 64 - DNA, 401 bp, Homo sapiens
tacctgtttgaactatacaggctataacatatgacttagagtcttttagttatgaccactcttaagattgatgactaagttgaagtatttattagttgttattatggtggattgataatcatgcattttaacatagtaaactaaagtctgtgtcgtagaattagctataatttactttttaggctggatgaggttactgaygcttgtaatcccagcacatcgaaaggctgaggtgggaggattgtttgagtccaggagttcaagacaggcctgggcaacatagggagaccctgtctctattatcataaataaaaacaaatttttttaaacaagtgtatatatgcatatgcttatgtgttgtaataataatatgtcccagagttgcacatctggaagagaag

SEQ ID NO: 65 - DNA, 401 bp, Homo sapiens
cccgagtagctgggattacagacattgcaccactatgccctgctaatttttttgtattttaatagagataaggtttcaccatgttggccaggctagtctcgaactgctggcctcaagtgatccacctgcctcggcctcccaaagtgctgggattatagatgtgagccactgtgcctggccctaattcccctttatgttttytctcacctctctggccagggctctgaagatattcctgggagaatgagggtacatgccacagagggagccttgggtttacagtcagcatagccctgcccatttccagctgcgtaaccaggagaagtggtggcttttctcagcctcagtttattctgtacagctggggtgagggtggtacctaagagaattaagtgaggtaa

SEQ ID NO: 66 - DNA, 401 bp, Homo sapiens
agaaagactattgaatccaaaaataataatcctttatatttgtacagtatttgcagttttataaggaactcttcacgttcattttctcatttgatttttctgacaactccattagggtagagagggcaggtattgttacctccaaattacagatgaggaacctgaaacctgagtgcttgtaagtcctacccagggtttcayagccactcagtgactgggactggagccccctagattttaccctagctcaaaagccagtgctctttccactattccgttttgatacccttggaaagttcactgcttatctaggaaagaaaaattggtagttcttgctgaggaacgtggaaatctgaaggagagacagatctgagagagtcacattggctagtaagtaaaac

SEQ ID NO: 67 - DNA, 403 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgcatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 68 - DNA, 401 bp, Homo sapiens
ttctactttgtacacataatatagaagagatcacctaagcctagttttgctaaccagaccctagacttaaaattagaggtcatgatgtctagccaccttcctctgtaggaacctctcgtgataccctgaaagcctctgcttaaatacttccagagaaaagtgaggaaggtaggggtggggatgaaggtttgcaggaactcrtgttgagtacctagtatctgcaagatactagagtaggtactttatcgccatctcatctgacttatgtatgagtgcaggttttataaccctcaagtttacaagtgaggaaactaagtttatataagtgtcacgaaacttgcttcagttcagatgtctagcttgaggcaaatctgaaattagaacctaggtccatcttcttg

SEQ ID NO: 69 - DNA, 401 bp, Homo sapiens
tccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaaggtgaacaggataggttaacttgtagtctatgtataagtttcygaagttgcatgcagaatttagcctatatgtgaatttttctggaaaaatggtcagtaactattgtcagattccttttttttttttttttttttttttgaagacaaagtcttgctctcttgcccaggctggagtgcagtggtgtgatcttggctcactgcaacctccacctcccaggttcaagtgattatcgtgcctcagcc

SEQ ID NO: 70 - DNA, 401 bp, Homo sapiens
ttatgcctgtttatacgatcactcgctgtagcagtatacaaaaaattctgttgatctgcattctctccagaatttggcactgccagatttttctttttgccaatcttgaggctaaaaaagagtatttcattgtgtttttaatttgcatttataatttgattactaatgagactaaacatctttttgtatatgtatgagccactttactgtggaataaatgtttttgtcatttattcatttttttctattttattgcttattgtttacttattggtttgtaggagttctttatagattctgcattctaatttttggccagtgtacgtttgccaatatattttcgtagtttctggcttgttttaaaattttcttcatgttatctttgatcaacaaaaattctt

SEQ ID NO: 71 - DNA, 403 bp, Homo sapiens
cttgggttttgatgaaagaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctctcatgcatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttatttta

SEQ ID NO: 72 - DNA, 401 bp, Homo sapiens
gcatgtgatgggtgaatgagtgtttcagtgaaatgacataagtctgtataatttggagggtaatgatgccttagaacaagaataaatctggagcgatggaaaggctccatattctagatgaatgcatgcttcctcttatgactctgaaaaataaaattaaatctttatttatacaaatccagtgaggggggaaggctacatggtttggcttaatgatatatttcagaacaggaatattagccttaacctctttcctcacattgcatatgatatttaatccatcatctttgttttaaacaaacaatacacaagctgttgctggcattggtataaagctgatggtccatctggagagcaggaatatagatcaggaaaataagagaattgaaattgggtgcaag

SEQ ID NO: 73 - DNA, 401 bp, Homo sapiens
aaggactggggtagaactctctttttcattttcttttaatcctgaagttacatcactgtgcagtcagctcaacttgtgtcattgcagtaggaagatatgtaggtggaaagctattccagaaggaggctggagctaccttccctgacaagaaaaaaaatccaagcaaatacatacataaaaagacactcaaagcatcatagratgttatagttataagggaccataggtttcatttaacgcacattttagcatccaggtcctacataaaatgtatattttgtgattgtgaatgggagttttccatatttaaaagttaagtttattaatgtgtcattttacccatcatgtatttgtggccatttttttaataccacgaatgggaacatactgtatcatagttt

SEQ ID NO: 74 - DNA, 401 bp, Homo sapiens
gaaacatacttcttcttagggacatttttaaataccaaatgagaatatgtttggtgggccaggtgcaggggatcacacctggaatcccagcactttgggtggccaaggcaggtggattgcctgaggtcaggagtttgagaccagcctggccaacatggcaaaacccagtctcttctaaaaatacaaaaaaattactaggcrtggtggcaggcacctgtaatcccagctactctggaggctgaggcaggagaattgcttgaaccgttgaggcggaggttgtaatgagctgagagtgcaccattgcactccagcctgggcaacaagagtgaaactccatctcagaaaaaaaaaaaaaaaaaaaagagaatatgtttggtagaaatctgaaagagaatttatgc

SEQ ID NO: 75 - DNA, 402 bp, Homo sapiens
caccattgcactccagcctgggcaacaagagtgaaactccatctcagaaaaaaaaaaaaaaaaaaaagagaatatgtttggtagaaatctgaaagagaatttatgctgaattgagaccatttggaaggcttcttgggtaaaactgatttgagttgtggatgaaggattgtttggaaatgagagaatgagcagaggccatggtggagagaagagttggagagcaggtgaaaggcgtgagcacagctgcagaagcagacatatgcacgatttgtcctagagcaggtcggttggcgagtttggttagaatggggggtttacatagcggagggtattgaatgccaatttaaagatctaggcagtaagaatcatgtagggtttttgaacagggatatgacatgcttc

SEQ ID NO: 76 - DNA, 401 bp, Homo sapiens
tttatttagatcttattcttggttacactgaattttatttttcaccaggttttgaacacttttgtgaagctgcttttgtaaattgtttttaatattttgtttttaactctaatttctagtatagaaatacagttactttgtgtatagtgatcttatattcagcgatatttacatatttttagccaataatgtattggtaarttcttttttgtttactatttcgataatcatatcaaatgcaaacagtgagaaagatgttcatttctattctatgcttttatcttccttacccttctcattgcactgagatctgcaactcagtgttaaattaacctgtgatagtgggcattcttgatttttttttcttgatttcctatttatttttaatccctgaatgaatc

SEQ ID NO: 77 - DNA, 410 bp, Homo sapiens; identical to SEQ ID NO: 55

SEQ ID NO: 78 - DNA, 401 bp, Homo sapiens
ataaagaattttattttccccagtagaccgggagctcctcaagggcagggacctttgcaagtctttgactccctagcactgaacccagcatctggcaaatcttatttcatgtgactttattttggctgaatggcctaaaaatgcgcttgtactgagcacctaatacatttaatttaatttttaaatttttattcaataatrtagacgtggagtccccctatgttgcccacgctggctcaaactcctggcctcaaggatgttcctgcctcagtctcccaaagtgctgggattacaggcatgagccactgcaccaagccctaatacatttaataaattgtaaggaggaagaacagtggaccgcagtgagatatggtctacagggaaaatgaagaacctcttca

SEQ ID NO: 79 - DNA, 401 bp, Homo sapiens
caggattatgaaattgtaaaggttactttctgctctctaattcctttactcgtaattttagttttcttaacggtataacttgatgcaaatatatacacagtagatactaactttcactgaagtgttttcgggagggaggggcactttacaagatgtgttgcctttagtttttccggtaaggagacaggaagacacagacasaggctcttagggcacatggaaagcgcctgcccctgtgccaagaacttaagagagagccggggatggaccctccttgctgtggctcctgaacagtgcagcctctcttctgatgcactcaccctggcgaggagaaccgcttgtgtggcgactgcttggcccaagagcgagtaggattgttgactcaactctcttcgtgtctc

SEQ ID NO: 80 - DNA, 401 bp, Homo sapiens
agggctaataaaaagcttgagtagtataaaaaggctgggggaggtgctgtggagcctttctaacgcctatgagaggcaaattaaaaccaattgcgaaacatggaaactggatgaatttcaatattgatttagaaattaattttattatgttgttctaaatataaaaataaaggctatacttattaatcttccagaatttgrcatgttaacacctatgcacaaagcattttatggatctttatatgctgggagacacaattatgggctgacttggaagttttggcagacctgggttcaaattctagcaacttcccctactagtttttttgtttgggggggttttttcggtgcacattacttcacctttgtgaaatccaatttcatcatctataaaatgagat

SEQ ID NO: 81 - DNA, 401 bp, Homo sapiens
gtggaccaaggtcaggttagttaatttgtttgcttttattggaataacttttcccattccaattcatcacttaattttaagtggttctactaaaatacccattaaagtcctttacatttctcacagactagggctatgacagattcataagacgtttcttttttcctcaaggcacaaaaggtagcttttctggtcatcaayctagtcttccttgttcctaatttcacaacagtggggcaacattttatataataccaatatgaactctaattgcaaagagactaaatgaaatcttcacacgaagcaagattaattaaaatataaaatgtgcttgactgtggtaaaacatttcttttaaaaaaaatagtatgacttttttttttttttttgctaaatctttc

SEQ ID NO: 82 - DNA, 401 bp, Homo sapiens
gttggtacttgtacccaagggaagagaaagttaatgagtagaaaagacagaatttatttaggaaaacgatggaattaagctctggacaaaacatgcatcagtgatgcataacatttttgttaattgggtcttaacaggtttctgactcatatggtaatcattgtagaagtgggtcttactagaccacgccagtggcaaatygcgaagctgtaagaactcttacctccctgtgtgtgtgcacctgagtgtgtgggggggcgtacgtttctgtagcttaatttaggttccacatacacttcagtggttaaacctgagctagactcaaatagtttgtttccattaagaaaatgaatcttttatgtggaccaaggtcaggttagttaatttgtttgcttttattg

SEQ ID NO: 83 - DNA, 401 bp, Homo sapiens
gaggtacaagtatcttaggctactggtccgggcaggctttgctgaggggctccgtgcagcttgctggtgcagccgagcaaatgggcctgtagccgactcttaatccaggttggtgctattcaaagagatcatctttcacccgagggatttctgggcatctattttgcggatcagaaagtagagaaagaaggtaactttgcygaaagctagtctggggagttagtagctgatacagatcagcatttcctaactatgagatttcataatattctctcttgtctcgattctgagtcactggtgcctgctgtggtggcattgttcatgaacatgtacagttattgggaagtgatcttgtctttcctcctgccttcaggcactgctacctaaattacaccccgaca

SEQ ID NO: 84 - DNA, 401 bp, Homo sapiens
ttaatctgtaaaacagaaataattcttaacacatttggttactctgaggataaaagtggaggaaaaaacatgcagaggatagtccggaactccccatgtagatcccattattaggtaactgaggtacaagtatcttaggctactggtccgggcaggctttgctgaggggctccgtgcagcttgctggtgcagccgagcaartgggcctgtagccgactcttaatccaggttggtgctattcaaagagatcatctttcacccgagggatttctgggcatctattttgcggatcagaaagtagagaaagaaggtaactttgccgaaagctagtctggggagttagtagctgatacagatcagcatttcctaactatgagatttcataatattctctcttgtct

SEQ ID NO: 85 - DNA, 401 bp, Homo sapiens
acgggcagttgaagaaggcatgtctgtaaaattgaagaagagatttcttagaatgacctcagtaacaagcttaaatatttaagtgtctggtgaaagtaggggtgggatagtctctgatgtgctacaagatcgaagaaggagttaagtccttctctagagcctcaaacccctgctcagcatgaaaaaaacaacagaaacccragttaacatctccttgcaatatctgatctgtttttccaatacatctgctcatcttgtttcaaaacaagtagctgtcaccattcttaaccctgtcgtccaaaccagaaaccgggcatcatctttgactggtcccctttactcagggggaaaaaaaaccatgtcttttaaagtcagcgcctataatactggtctttggttta

SEQ ID NO: 86 - DNA, 401 bp, Homo sapiens
atcatattatgtgacgggcagttgaagaaggcatgtctgtaaaattgaagaagagatttcttagaatgacctcagtaacaagcttaaatatttaagtgtctggtgaaagtaggggtgggatagtctctgatgtgctacaagatcgaagaaggagttaagtccttctctagagcctcaaacccctgctcagcatgaaaaaamcaacagaaacccaagttaacatctccttgcaatatctgatctgtttttccaatacatctgctcatcttgtttcaaaacaagtagctgtcaccattcttaaccctgtcgtccaaaccagaaaccgggcatcatctttgactggtcccctttactcagggggaaaaaaaaccatgtcttttaaagtcagcgcctataatact

SEQ ID NO: 87 - DNA, 401 bp, Homo sapiens
tttgtgaaatccaatttcatcatctataaaatgagataaataattctattcctgaagaggctttataagaattttagaaaattaaaatagtaagtatagaacacttggtacaatgcctggcacataataggtgttcagaaataggtaccccatatttagaaggaacttccactgagttaagagtggtttcaagcaaactgycctataaatatgtgagaattggattatgaatgtaaatattgtgtgcgtaatcttatggatcaagataagtttaagggaaaatgctagcggacagaaacttatagcttttggaagaaaaggtgactcaaacataagaagcaaattatttgaagcccacagtagccaaatggagagagtaggttgaaacatttgttaagtgc

SEQ ID NO: 88 - DNA, 401 bp, Homo sapiens
aagtctgttcttaattggttgattaatgtacgagaagtccttttcccccctccatctctacagatggcaaatggtaggtcccaactgtcattgttcacaaaaaaggttatggttcaaagtcaaagattcagagataccacaataatcaatcataggaacttgtctcagagtgcccagccaggcaaaagttaggcagagtartaatatttactgagaatctcttatgagtatttttttttggtgtgttctttattttatttagaaaatattatttaattaattgaaatgcctctgaatttagtgacaagcatttaaataaatatgaaaaataatggtcaaaaagttttctgtttatcggttttatcagatagtgctagaatacataattttaaaatgggtgt

SEQ ID NO: 89 - DNA, 401 bp, Homo sapiens
ctaggattacaggcatgagccactgtgccttgccttcatatgttatttctgatccactaggtttggaacctatcccaggacacctggccatatagagtagctatatcgagtctatattcagcaagtgcagggtaagctctgattccatggtccttccaatatgccataccaccgaggttgagaaaggtggtgttaacagtmcccatacgttgacataaggcctctgagaggccaaaggatatgccacagtattctttaaggtgcttaagcccttaatcatgaaatgttttcctaggccacagtaagaatctacttagtttacacacaattctaataaatccggttatctgtttttcaaatacaaagcagattgattcattcagcaaatattttctgaatac

SEQ ID NO: 90 - DNA, 401 bp, Homo sapiens
ttgcttttctctctccaggatccagcacctggcctggcacagggtacatgctcagagaacaagtctttgaaagaatgggtagatgtttattttcctttgtattagccattagctcaaggtctgcagctacttaattccaacctgggtccatttttagcagaagaaaaaagaataatgggactcagcatcaaggcgcacctgacacagagtcctcttggaaatgtgtgacctgcctcagtttagccactgcttttacttcatcctcatcagtcagagtatgacattgccttcccctttacctcttaattttggaatatttcaagtgcctctaaaattttatttaattaaggggcttccaaatctgcttgtagatattttattcttgaaatgcttgtggcatt

SEQ ID NO: 91 - DNA, 401 bp, Homo sapiens
taagttaatcatagctaccacttagaacttcttactcactagacatgtagctgaacacttcatatgtcattctgctttttgtttttttaaagacagggtccctcccactctgtcacccaggctggagtatagtggtgcagtctcagctcattgcaacctctgacccccaggttcaagcagtcctcccacctcagcctcccrggtaactggggctacaggtgtctgccaccacacctggttaatttttgtatttttttgtacagatggggtttcaccatgttgcccaggctggtctagaacttctggactcaagtgatctgcccaccttggcctcccaaagtgctaggattacaggcatgagccactgtgccttgccttcatatgttatttctgatccacta

SEQ ID NO: 92 - DNA, 401 bp, Homo sapiens
agaaatttaggatggagatttttgtttttaacctattagtcagcgtggcatcagagaaccacatgtgccccacagtcagtggcatgctcagtaaatatttgttgaatattatttaagtgaatgactgtttgctgaagaacaagatttctctgatgaccttgacaaacgtatgtttgtgattaagttaatcatagctaccayttagaacttcttactcactagacatgtagctgaacacttcatatgtcattctgctttttgtttttttaaagacagggtccctcccactctgtcacccaggctggagtatagtggtgcagtctcagctcattgcaacctctgacccccaggttcaagcagtcctcccacctcagcctcccaggtaactggggctacaggtg

SEQ ID NO: 93 - DNA, 401 bp, Homo sapiens
tgggttccagaaatctccctcctcagcctcccaagtagctgggattacaggcatgtgctactgcactcagactaattttttgtatctttagtagagacatggtttcaccatgttggccatgctggtctcgaactcctggcctcaagtcgtccgcccacctcggcctcccaaagtgctactattacgggtgtgagccaccgygcctggctagaattagtcattttaaattcactggggctgggcgcagtggctcatgcctgtaaccccagcactttgggaggccgaggtaggcagatcacttgaagccaggagatcgagaccagcctggccaacatggtgaaacctcatctctgctaaaaatacaaaaattaactgggtgcctgtaatcccagctacttggg

SEQ ID NO: 94 - DNA, 401 bp, Homo sapiens
gcaacctccgcttcctgggttccagaaatctccctcctcagcctcccaagtagctgggattacaggcatgtgctactgcactcagactaattttttgtatctttagtagagacatggtttcaccatgttggccatgctggtctcgaactcctggcctcaagtcgtccgcccacctcggcctcccaaagtgctactattacrggtgtgagccaccgtgcctggctagaattagtcattttaaattcactggggctgggcgcagtggctcatgcctgtaaccccagcactttgggaggccgaggtaggcagatcacttgaagccaggagatcgagaccagcctggccaacatggtgaaacctcatctctgctaaaaatacaaaaattaactgggtgcctgtaa

SEQ ID NO: 95 - DNA, 401 bp, Homo sapiens
taaaataggaaaaaaattgaagatggacatgtttcaatttcgtgcccttaaattagtgaattagtgagcttttttttgttgttgttgttgtttgagacggagtttagcccttgttttctaggctggagtgcagtggcatgatctcggctcaccgcaatctctgcctccctggttcaagcgattctcctgtctcagcctccygaatagctgtgattacaggcatgcacccccatgcatgtaatttttagtagagacagggtttctccatgttggtcaggctggtctcgaactcctggcctcaggtgttccacctgcctcggcctgccaaagtgctgggattacaggcatgagccaccacgcctggccatgagctttgtttctaactctttaaacttagtcaa

SEQ ID NO: 96 - DNA, 401 bp, Homo sapiens
ccagaaaagacgacaaacatgcaaaatggataaatgtaatcctttgtacttttaccacctacatttttaaactacagtaactctgatttctctagaaaggtcagaggtttcagacagaaagctttgtaattcttcaaagaaagacattttcagtttggctgtataaagactgattatgcatagctttttactttatgtgcrgttgaatcttggaggtgctggagtggggtggggacgatgaataagtgaagctgtgttttgagacattagaattggagggaaacagacagattggagaggaacatagattcatatttttgggctttttactatcaagtcacccagtctaaaactcaggacagagcagcattaaccattcattttgctttgtgatagagcat

SEQ ID NO: 97 - DNA, 401 bp, Homo sapiens
cttgagacaagtcagtgccatctcagatataatttcctcagaaatcaagattttagagcatttgatgtttttaaattagagtcctttcaactaatagcctgtatgaagtttttaaaacacttataaaaaatttatggtgattattattattttgagacagggtcttgctctgttgcctagctggagtgcagtggtgcagtygtagctcactgctgcctcaaacttctgggcccacacaatcctcctacttcagcctcctaagtagctgggactaaaggtgaatcccaacaccctgggctaacttaaaaaaatattttgtggagatagagtctcactacattgcccaggctggtcactgaactcccagcttccagtgatcctcccacctcagcctctcaaag

SEQ ID NO: 98 - DNA, 401 bp, Homo sapiens
taaatgaacttttattgatttcttagcttactgtatagacaaaaatcttacgtttaaaaaaggttttaaaattaatgtattttagagtttcagtaaaatgaagtgggaacaaaaattaacaaaaaagtgccatctcattggatccaggatatgtagtaatacacacacacagttctatgtctgtgtaaccatatatatatrtgtatacacagttctttggagaatgaggatatttcagtccaaaatgttatggttgaagaactaattgttctaaagatatttgcccctggccccagaaaagacgacaaacatgcaaaatggataaatgtaatcctttgtacttttaccacctacatttttaaactacagtaactctgatttctctagaaaggtcagaggtt

SEQ ID NO: 99 - DNA, 401 bp, Homo sapiens
tgtatagagcacagaacaaattatttgttccataattatttttaaaaagcagaaaatgtgtttgtaacatttaagaaatggcatgggattgtcatagaaactcattctgaacactgtaaataaaaatggttggggattagtcacagtaacatgtaggacatcatgaaattcaagacagatttctttgtctaacagtcagtwagagacatgaagcgaacttgatatagttcatgagatattagcagttcagttccttcagagaaagagaaaaccatgttgcttcatattaaataagccatctgtatacaaatctcacaatttacttgatgattttagcaatatttgatgtttcctttcagagtcttatttccaaataatgaaagcttaaaaaatttgaaaat

SEQ ID NO: 100 - DNA, 401 bp, Homo sapiens
tttgggctttttactatcaagtcacccagtctaaaactcaggacagagcagcattaaccattcattttgctttgtgatagagcattcgattaccaactctttaaactcttttggcaattcaaggaggttgcctatctgggtaccatttcttaggatttgctttgttggattttctaaattgtggatgtgagtttctttggktttgcattttttaaaaaaattttttggtcctttttaacttttccaagttttggagaatttttcatgttgggcggtgttggagaagtagagattgtagatgaccttcgttgtggcttactcctgttatttgggaaatagacgttcagagagcatctcacttgaggccacaccaacagttaagaggcaaagccagcatttaa

SEQ ID NO: 101 - DNA, 401 bp, Homo sapiens
aggagagacagatctgagagagtcacattggctagtaagtaaaacaggaaaagaccaagggtgtctagggacctggcctcctgccctggttcattcactgtgtgatcatgggcaagtcactgacttctccttgcttgagatttattttcttctcttttcttttcttttcttttttgacagtctcattctgttgcccacgcwggagtgcagtggcgtgatctcggctcactgcagcttccacctcctgggttcaagtgattctcctgcctcagcctcccgagtagctgggattacagacattgcaccactatgccctgctaatttttttgtattttaatagagataaggtttcaccatgttggccaggctagtctcgaactgctggcctcaagtgatccacc

SEQ ID NO: 102 - DNA, 401 bp, Homo sapiens
acaaggaagaaaagagtagggaaacctatttgtataggaaaatatagtacatttccattcaatatctacctataatgtgagagattatgccagacgcattgaaattgctgtctttaatcttcatcccaatcttataaattagcttttgttaccactgtttccttgatgaggatgccaagtccacaccaaatgacttgcctragatatgtattcattacattttggagttgagctttgaacccagctccgtctgtgttccatactcatttcttttccctgtatcagaagatccacccacactggccaactgagaagaaaaatagcagatgggcttaaatatgggtaaatgtatgataatgtacttagtgaagaaacacctaccaaactacttatagtttata

SEQ ID NO: 103 - DNA, 401 bp, Homo sapiens
ccacctgcctcggcctcccaaagtgctgggattatagatgtgagccactgtgcctggccctaattcccctttatgttttctctcacctctctggccagggctctgaagatattcctgggagaatgagggtacatgccacagagggagccttgggtttacagtcagcatagccctgcccatttccagctgcgtaaccaggaraagtggtggcttttctcagcctcagtttattctgtacagctggggtgagggtggtacctaagagaattaagtgaggtaatggatgtagggtccttggcatagtgactggtactcaatatcctctaaataaatattattctgtgttagggaaaagtgaatagagataaatgctaagggtagaggtgacgagagaagtggat

SEQ ID NO: 104 - DNA, 401 bp, Homo sapiens; identical to SEQ ID NO: 8

SEQ ID NO: 105 - DNA, 401 bp, Homo sapiens
gttgttgttgtttgtttgtttgtttttttgagacagtctccactccgttgcccaggctggagtccagtggcacgatctcagctcactgcaacctctgcctcccaggttcaagtgattttcattcctcagcctcccaagtagctgggactacaggcttgcaccaccgtgcctggctaatacagcttttttttttttttcttrattttatcataggtaagggaagacgatccaatgtgcagagaaggctcaggttttcattttagtctgcgggtgattgatttctttctttcaaggggctggttgaggaggtcagagtcttagaaagggagaagaaatcagggaaaaggagaaaagaaggaatgagatttatgaccctctggatcccagattttatgtcgcgt

SEQ ID NO: 106 - DNA, 403 bp, Homo sapiens
atttctttaccttggaactttagaagagggtctgaactgagcaaaaattagtgtccctgcctttttaacggctggacacttatcacaaagctgtgccaacatcagtgatggtgcacccacaaaggtgtttggtcttgataagcttctaaagaagcagactttgttgttgttttaaacagtaatgaactgtttcagtttcataaaaaaaaaagagacattctttcttaaatagaaaagggcagaaagtttatagagaataatgtctaacttgctaatgcagtgtttgcctttgctctgtggcatgtgtgtgtgtgtgtgtttatgtaggcatgcctacacggctgcttgtgttaatacttagtataaagccttaaaatggataccagattggctatgtaacctt

SEQ ID NO: 107 - DNA, 401 bp, Homo sapiens
atagtcagaggtctgtatgcctttgaaaatatccattcccttacctgtcctcagccagtaacaatttatgtcctgaagatagcaaggagtgctgatttaatttattttacaaacagtgtcctttcatgggagagagcaggctggtaaaattgtttcaagaatcagaacatttttcttggttatttataacataactcgacmatctagctacacagggcttcattcatttgagaaagactattgaatccaaaaataataatcctttatatttgtacagtatttgcagttttataaggaactcttcacgttcattttctcatttgatttttctgacaactccattagggtagagagggcaggtattgttacctccaaattacagatgaggaacctgaaacctg

SEQ ID NO: 108 - DNA, 401 bp, Homo sapiens
tgacattttgcagtttttgttgttgttgtttgtttgtttgtttttttgagacagtctccactccgttgcccaggctggagtccagtggcacgatctcagctcactgcaacctctgcctcccaggttcaagtgattttcattcctcagcctcccaagtagctgggactacaggcttgcaccaccgtgcctggctaatacagcttttttttttttttcttaattttatcataggtaagggaagacgatccaatgtgcagagaaggctcaggttttcattttagtctgcgggtgattgatttctttctttcaaggggctggttgaggaggtcagagtcttagaaagggagaagaaatcagggaaaaggagaaaagaaggaatgagatttatgaccctctggatc

SEQ ID NO: 109 - DNA, 403 bp, Homo sapiens; identical to SEQ ID NO: 106

SEQ ID NO: 110 - DNA, 402 bp, Homo sapiens
ctggaatttaattatcagtgccataaataatcttgtgaatggaagcagtgtatttggcagtgaatttctgcttcctaaagagaaaggaacctttagaagttatttgaaataattctgtattagccacgatcctggaggcaaatggtcacagaagcagaggatggtatccccagagaaaagtgggttttagatgagtcagataatgtggatatgtgctggtgacgaatgacatgaaggttggatgtattttttaaaatacaaatttaaagcaggctgtatttagaagtttatttataattggttttaggataaagccagcctgttgatgcataacagagttgatcttttggttccattagcacccttgaaatatttaacaagaagctgactttagcatctgag

SEQ ID NO: 111 - DNA, 401 bp, Homo sapiens
tctcattctgttgcccacgcaggagtgcagtggcgtgatctcggctcactgcagcttccacctcctgggttcaagtgattctcctgcctcagcctcccgagtagctgggattacagacattgcaccactatgccctgctaatttttttgtattttaatagagataaggtttcaccatgttggccaggctagtctcgaactsctggcctcaagtgatccacctgcctcggcctcccaaagtgctgggattatagatgtgagccactgtgcctggccctaattcccctttatgttttctctcacctctctggccagggctctgaagatattcctgggagaatgagggtacatgccacagagggagccttgggtttacagtcagcatagccctgcccatttcca

SEQ ID NO: 112 - DNA, 401 bp, Homo sapiens
ctttgttttaataggggactcttagagacaaggaagaaaagagtagggaaacctatttgtataggaaaatatagtacatttccattcaatatctacctataatgtgagagattatgccagacgcattgaaattgctgtctttaatcttcatcccaatcttataaattagcttttgttaccactgtttccttgatgaggatrccaagtccacaccaaatgacttgcctgagatatgtattcattacattttggagttgagctttgaacccagctccgtctgtgttccatactcatttcttttccctgtatcagaagatccacccacactggccaactgagaagaaaaatagcagatgggcttaaatatgggtaaatgtatgataatgtacttagtgaagaaa

SEQ ID NO: 113 - DNA, 401 bp, Homo sapiens
aagggtgatttagtgtgaaagtactagggtctttgactgagaagtcaccaaaaatcaatggctgtattgttttgaatgttatctgggcttgcaactgagaataggacctgggggttttactctttttattccttttcattcgttcttcactctctatctgaattgcagtcgtggtcctctggcaggcagcgtctgtatgcraccggaaggcagtgctgaatagtggggtgatgttggcagagcactttccaggcatgctagcacctagcgcacaagcagagaactttgcttctgccaactgtgacttcagaacccaggtctgttcctgaacctggatgcccagctgtcagtgtgtcgccatatgtcaccagcacatggcaccctaattaaaatttcaaatt

SEQ ID NO: 114 - DNA, 403 bp, Homo sapiens
tcctggctaacaaggtgaaaccccgtctctactaaaaatacaaaaaattagccgggcgcggtggtgggtgcctgtagtcccagctactcaggaggctgaggcaggaggatggcgtgaacccgggaagcggagcttgcagtgagccgagattgcgccactgcagtccgcagtccggcctgggcgacagagcgagactccatctcaaaaaaaaaaaaaaaaaatctagagttgaaatttttctcttacatttccttttccctctagatcaatcccaattaaagtttcattgcaaaagtttcacaaactcatattggcattaattattattggtgtgctggtgaaagtcctaaagtgagttcattaagaattaaaaaccttggctgggcgcggtggctcacgcctg

SEQ ID NO: 115 - DNA, 401 bp, Homo sapiens
catcctggctaacaaggtgaaaccccgtctctactaaaaatacaaaaaattagccgggcgcggtggtgggtgcctgtagtcccagctactcaggaggctgaggcaggaggatggcgtgaacccgggaagcggagcttgcagtgagccgagattgcgccactgcagtccgcagtccggcctgggcgacagagcgagactccrtctcaaaaaaaaaaaaaaaaaatctagagttgaaatttttctcttacatttccttttccctctagatcaatcccaattaaagtttcattgcaaaagtttcacaaactcatattggcattaattattattggtgtgctggtgaaagtcctaaagtgagttcattaagaattaaaaaccttggctgggcgcggtggctcacg

SEQ ID NO: 116 - DNA, 406 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 117 - DNA, 401 bp, Homo sapiens
taatgaatccgcttgactttaattctagcccagatgtaatcttttatagcttgatgatttgttctcccaggcactctgatgaaatgctataaactgttcgctagaatccagtgaaaatagtttttattcctcatttctgtttctaattgttgagcaattagattgattcttgatctcttacaatcaccataaaattgtacyatggcaaagattcttaatcatgatctgcatcaccctacaaatagaaaacactgaaacattttgagaaaaaaaagaactttcttaattcatttaattagaaaacataaaaatacaagaagaagaaataaggggtattggtcaaagggtataaaattttagttatgcaagacaatttctggagatctaacgtaaagcataat

SEQ ID NO: 118 - DNA, 401 bp, Homo sapiens
tgagttagtacagctactttaaataccagttgtgtagattcccacacttttctaccaatggagaggtttacacaagcataatttaagttacaattacactaattaacatctcatttgcataaattgttgaagtcaaaacaacaaagaattgtctaagaagcttaatcctatttgtccaaaatagaaagatttttttttttwaaaaaaaaactatgctctaaaaattggcagcttaagtatgtctttagtatgttgagctgtgtccttttaaaaataaatgttttcaattttcttaataatatatttctctattctttttagaatccttcatttttagtatacttttaaaattgaacacacacaacactttggttcctaaagtaatgataacaagaaattaa

SEQ ID NO: 119 - DNA, 401 bp, Homo sapiens
aagaaagaaaaaaggaaggaaagaaagagagagaaagaaaaagaaagaaagaaagagaaagaaagaaaagaaagaaagaaagaaagaaagaaagaaagaaagaaagaaagaaagaaagaaagaaagaaagaaagaggaaatctgtatgatgtgcaggttgcatttgactaagctcctgggggaacaacctcaccgtcctcygcttagggtcaatgaggctgcatcaggcttcctctctctctttctggaaacatgtttgatgggggaagaagaaaatggcttgtgatacagtaccaaaggagcaggctggggatacctgagagcagcattctagaactcagatgtgtccaggggtctggcaggtaacaaaagcctgaagccgacctagtatggtgagattg

SEQ ID NO: 120 - DNA, 401 bp, Homo sapiens
gaatgaaacttttccaaccacttctggcaaggttcacaattgcatatggataagatgcaaagtgtaagataatttgaatgaattacggactatagtgaaaatgctgagattcgaaggctgaagttctagctggcagaagtttgcaacacacagaacttgtagataatatcagcgagctgcaataaagggaaaggaccctarcagatacattagtgtcttctcaattgagctctgaaaagataaatttatatttgggaatggtgcatatatgagggcacctgtatatccatgcaggcctagggcacagtcaggtatttaacaaagttggcctctgttggttcatgaggtattatgacattaagagagtttttttttttctttttttttcggcttacatattc

SEQ ID NO: 121 - DNA, 401 bp, Homo sapiens
ttgtcaactgtatctagttccatactgtatgtatttttatatctctggtaagtgcttatggtggtcccaaataacttgtgagtttccagacatagacagttctcagagcatgtgtattcatataaatttttaaatggctattgctgtagtgctctacaggttaaaaaaagtgaggcgaaattgaaacatttaaaaaatacygtactatttaaaaatttgtatgaatgtctgtgtagcagctgccattttcaggctgtctttgttgctatggtttggttgattttatttccttttgtaattttgccagccagtgattgggtagtacattgcattcatgctgttggggaagatatatctgtggtgagtcagatagcttgatctgagtagtaatttttagtagg

SEQ ID NO: 122 - DNA, 401 bp, Homo sapiens
aattttattttccccagtagaccgggagctcctcaagggcagggacctttgcaagtctttgactccctagcactgaacccagcatctggcaaatcttatttcatgtgactttattttggctgaatggcctaaaaatgcgcttgtactgagcacctaatacatttaatttaatttttaaatttttattcaataatgtagacrtggagtccccctatgttgcccacgctggctcaaactcctggcctcaaggatgttcctgcctcagtctcccaaagtgctgggattacaggcatgagccactgcaccaagccctaatacatttaataaattgtaaggaggaagaacagtggaccgcagtgagatatggtctacagggaaaatgaagaacctcttcaaaaatg

SEQ ID NO: 123 - DNA, 419 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgagtgtgtgtgtgtgtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 124 - DNA, 402 bp, Homo sapiens
gtctttcagaagtgagttagtacagctactttaaataccagttgtgtagattcccacacttttctaccaatggagaggtttacacaagcataatttaagttacaattacactaattaacatctcatttgcataaattgttgaagtcaaaacaacaaagaattgtctaagaagcttaatcctatttgtccaaaatagaaagattttttttttttaaaaaaaaactatgctctaaaaattggcagcttaagtatgtctttagtatgttgagctgtgtccttttaaaaataaatgttttcaattttcttaataatatatttctctattctttttagaatccttcatttttagtatacttttaaaattgaacacacacaacactttggttcctaaagtaatgataa

SEQ ID NO: 125 - DNA, 401 bp, Homo sapiens
gctgtttacttagattgttttaaaaggctagctgtgtggaggaatgaggaaggaagggaggcgggagtggctggagtttttttttttctctgttgacctaatgaatgcagttcttaaaaaataataaatatttatatgtggtcttaatttttaatgagtctcataaactggctggtatagaaaagtgtatgaaaagccatwtatattaaactatagttattataaatacatgcatatgtacactagaatgttttatgaggaagggaacttactagtcttgttcactctttgttttcccagtgtctagaacagtgcctgacatacaactggtactcagtaaatatttgttgagtgaacaaaatgaatgtaaattgacagcattgcctagtgggggtaccatt

SEQ ID NO: 126 - DNA, 401 bp, Homo sapiens
gtatatgtgtgcatgcatgtagataagtgtgtgcatttgcacacataagagttttaagctgctcctgtcatttattgatggtcaaaggtttcttttggctattgctggactcttaagattgtcttgtaattgtctttttgttgttgttgaaaattaagggtgtatattaaaggtagtttttacccagatcttatatgtgtratagctcacatctgtaatcagaaacttactgtttaatggccacccaattgccattagcttcctagagggtgatttaataaactatcttctttaaaactcatttaaaattagagacatgtttgcatacaatggattaatgacgttttcatactaacccacaaaagtctgctgcactttcttttgtaggcctaacattcatt

SEQ ID NO: 127 - DNA, 401 bp, Homo sapiens
ttggctgaatggcctaaaaatgcgcttgtactgagcacctaatacatttaatttaatttttaaatttttattcaataatgtagacgtggagtccccctatgttgcccacgctggctcaaactcctggcctcaaggatgttcctgcctcagtctcccaaagtgctgggattacaggcatgagccactgcaccaagccctaayacatttaataaattgtaaggaggaagaacagtggaccgcagtgagatatggtctacagggaaaatgaagaacctcttcaaaaatgatgatcttcaggcctgtaatcccagaactttgggagccgaggcgggtggatcacttgagcccaggagttcaagaccagcctaggaaacatagcgaaaccacatctctaccgaaaa

SEQ ID NO: 128 - DNA, 401 bp, Homo sapiens
tgggtgtgattcaaatcttgtctgtactgctgatgggctgtgtgactttgggcaagtagcttaacttctctgagttcccctgtctctgttttttcatttgtaaaatggagtggaggggacaatattaacttgcaggatggcttgatgatgagaaatgataaatgtcttagtctatattagatcttcagtaaatggtagttgtttgaccactgttactgcaatgagccaaggtggctataagcccttcagtgtttcagtaaggacaagcttacaggtaaccaccaagatcagggcagaacagctgatttaggtctaaacaggttccatcgtgtgtcttcaaaaaggttttcctttttttcctctggagaaaattcagactggtttaagaaggaaactgagag

SEQ ID NO: 129 - DNA, 401 bp, Homo sapiens
gattaacaggggccagcagtgtgacattgccattcaaaaagctggtgctgtagttgggctgtggtctaaccacagctgggtttttttttttttttttttttttgcccagttttgggcacagaatgctaagaggaacgccaacagaatggagactcgatgccaatgaattgggaaatcatattatgtgacgggcagttgaakaaggcatgtctgtaaaattgaagaagagatttcttagaatgacctcagtaacaagcttaaatatttaagtgtctggtgaaagtaggggtgggatagtctctgatgtgctacaagatcgaagaaggagttaagtccttctctagagcctcaaacccctgctcagcatgaaaaaaacaacagaaacccaagttaacatctcc

SEQ ID NO: 130 - DNA, 401 bp, Homo sapiens
aaaaaccgaaattgtttactgcctccaggatgttggtaaatatctggctgtattttttttttttttggcgggcgggggcggggggaggcaatgggtaggagaaagctagtatacagtgtcccaatttacattataaaatgcaaattttgtactccagaataagaaacaacttagagtagacctgtattgttccaaaactartgagaaattatcatgttttctatgtgtagaaaagttttctcttcgcaatggctttacatattttctgtagggaaaaattctatttataaaatagcctgggatatggagaactcagtgataaaaggggcaagaacattttcagattgttttaccctgctggaatgtagcatgggaaagctccttccaaaaggctattacct

SEQ ID NO: 131 - DNA, 401 bp, Homo sapiens
ttcgtgtgcatgtgcgtgcacatccaggctgcacagagtgctgtttgagcaatgggaatgatttcttttttgaaatgctagtattttaaatacagtgtaattatacagataaggttttcagtgcatatagaatattataatttttataaataaatcgcattagattgttacagtttataattttatgccaaattaattatraaaattattttaatttttaggtaacttcctacattagtttgaggcaacattgcatatccaaaatatattcaatatattcttttagcagctacatgtatgtgtgtttacattcaaatctaggactatacgtttttgtaattaaactttttcagtaacttaataaacaccctagtagaaagttgtgcagttacttcctgtga

SEQ ID NO: 132 - DNA, 402 bp, Homo sapiens
aaaacttcagaggggaaactgagaatgggactcggcttgcttctcctggtgtgggttcaggccgccattttaaggagccagtgaagggcgacgttccgctccttacatggcggctgtatttactcggccgcagccaatcagccggcagtgccaagccacgtgacatgccacgagggcacgcacagccatttccttgtttctaaaaaaacttgctacctccacagagtactttaccttgttttgcatgccaaatgttcttgctgaatgtgtctagcagactggcatttgtccataaagttattttagtaggtaaaaagtctctgagcacttgagctttgtgcattctttatgtaaaatggatttcccttcttggccagaggccaagggtacagcacactcgct

SEQ ID NO: 133 - DNA, 401 bp, Homo sapiens
ctcgcgggcacccggccgggccggcgcgggagcgggaaagggtgcgctatgcctttaacacccgcgtacagtaggcatgtatagtggagtgtagggaaactctaggcggggttaaagttcagctcatggagcggcaatagcgctggctggctggctgcagttgagccgacttggaaatgtgaacgcaagaagcaggcttgatttttttctccccccttctctctctctctctctctctctcttcctctctccctctttctcctctctcacccacactcacgcacacctccaaaccgcacacccagacgcacacgcataccccagcgcccggcagttatgtattctccgctctgtctcacccaggtaagccgcggcgtggatgcggagggcttgggggccgg

SEQ ID NO: 134 - DNA, 401 bp, Homo sapiens
ttgtgcagttacttcctgtgatttgtaattattaataaagtgtgtttggaaaaagaatatacactgtatattgtatacacaatatacacaatatacactgtatattgtaaaactaatatacagtgatatttaaattaggagagtaaggatcaccctatcaaactctagctttaagctcaaggtggattttttggctgatcragctattgtaaattattgatatatggcatatttttagaaattttaatttatcttttaattcttcaaaaaagatagcagtagcagtcaacaaatttgagataaatattgattttacatccttgagtgttttctgtgtaacttgaggatagtaacaatatcctttacctagaatgtagtaacaaaatcctttatctggaatg

SEQ ID NO: 135 - DNA, 401 bp, Homo sapiens
cagtggcatgatctcggctcaccgcaatctctgcctccctggttcaagcgattctcctgtctcagcctcccgaatagctgtgattacaggcatgcacccccatgcatgtaatttttagtagagacagggtttctccatgttggtcaggctggtctcgaactcctggcctcaggtgttccacctgcctcggcctgccaaagygctgggattacaggcatgagccaccacgcctggccatgagctttgtttctaactctttaaacttagtcaagattagttctttttttttttttttttttttttgagacggagtcttgctctgtcacccaggctggagtgcaatggcatgatcttggctcactgcaacctctgcctcctgggttcaaacaattctcctgcct

SEQ ID NO: 136 - DNA, 402 bp, Homo sapiens
tccttctctagagcctcaaacccctgctcagcatgaaaaaaacaacagaaacccaagttaacatctccttgcaatatctgatctgtttttccaatacatctgctcatcttgtttcaaaacaagtagctgtcaccattcttaaccctgtcgtccaaaccagaaaccgggcatcatctttgactggtcccctttactcagggggaaaaaaaaccatgtcttttaaagtcagcgcctataatactggtctttggtttatctccaataactcgattgttaacagcccttgaaggggaggcaatactgttaaacttgataatttctaaagagttttgagctatttagcacgaagtgatgccaagaaaaaggaatactaacattactcacagcagagggaaaaatttt

SEQ ID NO: 137 - DNA, 401 bp, Homo sapiens
ttgatttaatgcaccaaagaggatctttaactcttggtctacactagtgcaaataaaggttaaatctctcactaaccatcatgaactagcctaataactcacgagaggccctggaaaaagaaaccgagagagcaggtgtgcagacttgattaaaaggtgcaaaacccgcacaaccgcaggcaaaggtactttatcctcacrttgcccagtaccggctgcgaggtgcgcaagcaggggaacttgtactgcgccaacagaacgattccgagagccgggcctagtaacacaggggcttttcttcagaacggtgtccagaccggagcttgcgccgaatgtaggggctcctattggccacgcgccgtaggggaggagacgactggcggggagacagactggaggag

SEQ ID NO: 138 - DNA, 401 bp, Homo sapiens
aaatgatagatggatattctttaaaaatttttttttctatgcttgtatacattctttaatatgagcagaatcaaattgcaataaactgatttttaaaaaattcatttattcaagagatacttgggctgggcgcggtggctcatacctgtaatcccagcactttgggaggctgcggcgggcagatcacgaggtcaggagatygagaccatcctggctaacacagtgaaaccccgtctctactaaaagtacaaaaaaaattagctgggcatggtggcgggcgcctgtagtcccagctactcggcaggctgaggcaggagaatagcgtgaacctgggaggcagagcttgcagtgagccaagatcgcaccactgcactccagcctgggcgacagagcgagactct

SEQ ID NO: 139 - DNA, 401 bp, Homo sapiens
gtgagaactcactatcacaagaacagcagcatgtgggtaactatttccaagattcaattacctctcactgggtccctcccacaacctccatgtgggtattatgggaactacaattccggaagagatttgggtagggtcacaacaaacgacatcagcatatctatgacaaaggattaaattcagtttaattcaacaagactkcactggacccttattctaagtaatacaagcataacttaagaattattcatccaacaaatattgaaaaaacaacacatgtgacttactgcgttttgggagacacatagttgatttcgatctagataactctctcaaggaagctgcagtgtggtaggaatgataagacttgtacatagataagtaacaccaatgatagaaaa

SEQ ID NO: 140 - DNA, 401 bp, Homo sapiens
ttttaattattttatgttatacaatttaagtcatggaaagggggatgactgtattgtatcttttaagtataatgtatagcctttaatattcttaaagtggatgttagttaaggacaatttttagttgagagagagtgaaagagagagagataaggggggcagagaggattccattacattcagcacagtatgaaactaagtcaaaggagttttgttaattaaattcaattgccatccattagaccagtggaatgagatgactctgcctggtgctgacacagcacaggtatgcaatttggctaaatggccatttccaaaccatagcacacatttgtctacttgttcacttttttttttttttacatgagagtttttactcttagaaaagtcaaagagtaacc

SEQ ID NO: 141 - DNA, 401 bp, Homo sapiens
aactctgcttttcccagggctctttgaactcttgttttattaggtaatatgctatgctgttattttattgctgtttaaaaaaccttctcttttgggaaagaaaacatgtgaattctttggtttctagatagaaattagcaatcttttgctcggaatgtaaaagtatgctgtattatcacaaactgaccctcctcctccccmagaattctagggagtaaaatgcgtgcacaggaaatcagaaatggatcaacagagttataggttttaaaaaaactgccttgcatttttgccctggaattaattttcagtgaaaattaaaatattttttctgttcatctggaatcagtttttacttcattagcaggaaatggtaaatatacattcagcacaaatagagtttt

SEQ ID NO: 142 - DNA, 401 bp, Homo sapiens
gactgtgtctcaaaaaaaaaaaaaaaaaaaaacacatactcatattaaaaaaaatgtgtgaatgttcattgcagtattattcataataaccaaaaagtggaaacgacccaaatgtccatcagctggtgagtggatagacaaaaatgatatgcatacaatggaagattattcagccagaaaaagaactgacgtgctgacacrtgctacaatgtagataaaccttgaacagttgtgccaaatgaacgaagccagtcacaaatgattctacctatatacaatgtccagaatagacaaatctacagagacagaaagtagattcgtcactgtcaaaatctgagaatgacgactaatagttacaggttttctttgggaggtgataaaaatgttctagaattcgatac

SEQ ID NO: 143 - DNA, 401 bp, Homo sapiens
acgagaagtccttttcccccctccatctctacagatggcaaatggtaggtcccaactgtcattgttcacaaaaaaggttatggttcaaagtcaaagattcagagataccacaataatcaatcataggaacttgtctcagagtgcccagccaggcaaaagttaggcagagtaataatatttactgagaatctcttatgagtatttttttttggtgtgttctttattttatttagaaaatattatttaattaattgaaatgcctctgaatttagtgacaagcatttaaataaatatgaaaaataatggtcaaaaagttttctgtttatcggttttatcagatagtgctagaatacataattttaaaatgggtgtaacacagaaaataacattcttaatatatt

SEQ ID NO: 144 - DNA, 401 bp, Homo sapiens
tattatcacaaactgaccctcctcctccccaagaattctagggagtaaaatgcgtgcacaggaaatcagaaatggatcaacagagttataggttttaaaaaaactgccttgcatttttgccctggaattaattttcagtgaaaattaaaatattttttctgttcatctggaatcagtttttacttcattagcaggaaatgstaaatatacattcagcacaaatagagttttctttatgggacacaggatttgtactccataggtagatgtgtaacataaaagaatctttctgctctccttgtaattttctccctcttaccctcaataagttaaaagaaaggaaaattttttaacctaacctgttaggaaccattttagtgtagcctctagtattttgataa

SEQ ID NO: 145 - DNA, 401 bp, Homo sapiens
tgatcaagctattgtaaattattgatatatggcatatttttagaaattttaatttatcttttaattcttcaaaaaagatagcagtagcagtcaacaaatttgagataaatattgattttacatccttgagtgttttctgtgtaacttgaggatagtaacaatatcctttacctagaatgtagtaacaaaatcctttatctrgaatgtagtgcaaaagatggtagttctaaggcagagagtgtacaggaatagggagaaatagttgttctggagtaaacctttgaatggaagtcagttcatttatggctaagaggttgagtgtgttggtaagaaaatttcccaatgcttctggcttggattttccttgtcttcttttccaccctgtgatgaatagcattaat

SEQ ID NO: 146 - DNA, 401 bp, Homo sapiens
gcctttgctaattttcaagtgacattaggcctaaaagctaaatcacagtgagccatcttcactttttgcagatgaggaattttctaacattagaagtatttttagcagtcttaaaattcagtttgaaagattaataagtgaccacaatttgtcaggtaattgctttaattgttaattagccagtgacaaagcaagttgctrttgtcctaatggcagcagtatgagtacttgttagttttaaggtagaaagagaacacttttgcacggtaatcttttagtgcagtagttactgattgcttccatgctgctaccattattacctttacttaattttgcttgcatcaggaatagcaaagatcctttcaatcactgaaggagctgtttcagtgtctcggaagcct

SEQ ID NO: 147 - DNA, 401 bp, Homo sapiens
agtaatcatgcatcaggttgatacctggtgagtagatatttgtatttaatgctcttaaattttaacagccttgcctctgtatgccctgctcatctgtgtcttaccaactttgatattgtttgaaaaataaacatttgaaggaagaagtttgcatatttagagggttggtagttcattacctgcatgtaggttatgcccacrtcgcaaagaaatctgatctatatgggtaacatatttggataatcaaggccaaggtaaagggattcattcccccttcttgaattttatggtttctgtatgtcaaaccagatttcattggtttaaatcttgtggtgtacacttggccattgatttggtttgagctctgtaatatttgtaattatgtgagatgacaagattga

SEQ ID NO: 148 - DNA, 401 bp, Homo sapiens
aaatccctccatagtgatggaagaatgagccccagagagaagaatgtttctaatgaatcactggattgtgatataggattaacttggtgtccctaataccatttttttttcctcctgaaagtttaaggtcttatgtttaggaactagtttctctccaccttaatcctttattgtcaagtctgcaataatgttaagaacaggaaaaaaaaatgtagattcctggataggcacagtttttatattaatgtaactatataggcatagtttttatattaatgtaactatacagcacctatttttgtgttttactattacttggcagacatcttgagtgttttacaaggttatcgtatatttcactaataatcgttgcttgataatttggtgtcctgacagactgc

SEQ ID NO: 149 - DNA, 401 bp, Homo sapiens
aagtccttctctagagcctcaaacccctgctcagcatgaaaaaaacaacagaaacccaagttaacatctccttgcaatatctgatctgtttttccaatacatctgctcatcttgtttcaaaacaagtagctgtcaccattcttaaccctgtcgtccaaaccagaaaccgggcatcatctttgactggtcccctttactcarggggaaaaaaaaccatgtcttttaaagtcagcgcctataatactggtctttggtttatctccaataactcgattgttaacagcccttgaaggggaggcaatactgttaaacttgataatttctaaagagttttgagctatttagcacgaagtgatgccaagaaaaaggaatactaacattactcacagcagagggaaaaa

SEQ ID NO: 150 - DNA, 401 bp, Homo sapiens; identical to SEQ ID NO: 143

SEQ ID NO: 151 - DNA, 401 bp, Homo sapiens
caagtgattatcgtgcctcagcctcctgagtagctgggactacagatgtgtaccaccacgtccgactgtccgactagtttttgtatttttagtagagatggggtttcgccatgatggccaggctggtctcctcctgacctcaggcagtctgcctgcctcggcttcccaaagcgctgagattaaaatagaatcgcttgaacmcaggaggtggaggttgcagtgagccaagctcgtgccactgcactccagcctgggtgacagaacaaaactcccatctcaaaaaaaaaaaaaaatgccaggtagagcagctcacgcctgtaatcccagcactttgggaggccaaggtggatggatcgcgaggtcaggagatcgagaccatcctggctaacactgtgaaaccc

SEQ ID NO: 152 - DNA, 401 bp, Homo sapiens
atagaacaatgcctagcacatagtagagatacataatcactactactactgctaccagtacaacagcaggtcttatggacctaaggtcatataacttagtctcttccaagattcttgaaatgatttctcaaaacaagagaatataaagaagaaacgttatgaacaaatggtaaataagaataaatgttagtaataaatggtaaaaaaaaaaaaaggatatgaaagccaatagttacatgttctttcctgttaaagctattttacaaatggaaggaagcaaatttactttttcctcttgaacccgtgaactttgaaaatcttctcatctatttgactgagtagtatggtcttttaaatggtatataagataagaagtattcaaaataaagatatagccttta

SEQ ID NO: 153 - DNA, 401 bp, Homo sapiens
gtgggcaatctgaaaaggtgacttgaacatgatgagcatgccgctgtttaaaatcttctactggttttccatcacctacaaaacaaacttctgattctgtaactcagtgcacaaggcccatccttttctctcatctcccctgcagctctggccaccacgccctcctatgtcccctcctttgtctccttattctagccataycaggttgctaaaatttacccaagccggccacactgttccaaacttgaggaccttcgcttttgccatttcttctgtacaaacactgatcccttctccttcttcacccactccctcaatattcaaaactcacctatggcaaggctgaacctttctccaccaccccatgcctccctccctcacaatacacatacatgcacaca

SEQ ID NO: 154 - DNA, 401 bp, Homo sapiens
aatttttggttcactcccatggaaatcacttgcggataaccaggcagttattaaacatacaaggcttgtggaaagacccattcattactgagtccctgcaccagagtttctgattagcaaacattgacatcacagagcctggcacaagaggccaccataaatcctgtcacttagcaataggataagtaaggcagcaccttyggaaggacacaatagaaccgcagatgtagcacacaggtgtttggaaatatatcaatattgaaaatagggccaggcgcagtggctcacgcctataatctcagcactttgggaggctgaggagggcggatcatgaagtcaggagatcgagaccaccctgaccaacatggtgaaacctcgtctctactaaaaatacaaaaatt

SEQ ID NO: 155 - DNA, 401 bp, Homo sapiens
ttgtggcattcttcattaggactgataggcaatttttggttcactcccatggaaatcacttgcggataaccaggcagttattaaacatacaaggcttgtggaaagacccattcattactgagtccctgcaccagagtttctgattagcaaacattgacatcacagagcctggcacaagaggccaccataaatcctgtcacytagcaataggataagtaaggcagcacctttggaaggacacaatagaaccgcagatgtagcacacaggtgtttggaaatatatcaatattgaaaatagggccaggcgcagtggctcacgcctataatctcagcactttgggaggctgaggagggcggatcatgaagtcaggagatcgagaccaccctgaccaacatggtga

SEQ ID NO: 156 - DNA, 401 bp, Homo sapiens
aagtcaggagatcgagaccaccctgaccaacatggtgaaacctcgtctctactaaaaatacaaaaattagctgggcgtggtggcaggcacctgtagttccagctacttaggaggctgaggcaggagaatcgcttgaacccaggaggtggagattgtggtgagccgagatcgtgccactgcactccagcctggtgacaaagsaagactctgtctcaaaaaaaaaaaaaaaaaaaaaagactgaagtgactaagttgcttgtattttccagaaataaacatttcccataatgaaattccaaaccttagaccctgtagtggaagccacaaggccaagacaccactcccttagacacccctgtgatcttcagggcctctcctttggtctcctggtcacgtcacac

SEQ ID NO: 157 - DNA, 401 bp, Homo sapiens
tggaagccacaaggccaagacaccactcccttagacacccctgtgatcttcagggcctctcctttggtctcctggtcacgtcacactcatgcaaaatctgtcagggacactgaaaggttttatactcaggtgtcttgcccatctcaggcgatggtaacccatcagggttatggcacaggccataaacctgggagtcacccytgactccctctctgttctctctgttctcacagtccacattaatacaacaggaaattctatctctatctcctaaatatctctatctcctaaatatccccagaatccaaccatgtccactactcccatccttatccaagctgccgtcctctctcatctggattaggcgactgcctctgaattaggctcctgctttcattctt

SEQ ID NO: 158 - DNA, 401 bp, Homo sapiens
taaatgctatctaaatatttgtggaataaatacatcattgttattatcaagaaaccaatgacttatagagccccccttctgctgtgcattttgaaaggcccaacaagggagtgatttttgccccagtggcataacactaacggtcaggggcaaaggtgatattggggtttcctgatatctccaaaatagtaccctataagmaccagccctttctctctgctgtacttctggctttgcaggacccaaaccagccccttgcttgctgaaaaggtccccagcattctgacaacatataacaaaggtgtgggagggagtggggccacgctgaaaacagctgacccagtggactggctgctgccactggatctatttcccagtgtctcggaagttgctgacttcag

SEQ ID NO: 159 - DNA, 401 bp, Homo sapiens
gatgtatctgtctcctcccaaaactctgttttcatgaccctgcctgcatgttccttccccgtcccatgaccttagattcccagagtcctctgggagctctaaaaaatacaacatgaacacctgatattgatacagtgagttaccctttatagcactctgagtagatagggtaggcattgttaatctcagtttagagttcawgaaaccaaatatcaaagatattaagtgaatttcccaagatcacgcagctagtaagcaggggagcttggattaacccaagtctcttaattcctgatccagtgctcttttcatagcactaagtctctgttatcattaatttcttcacttgcataaagccaagatatgccaggtcactcctgaaaaaaaaatgacctcagctc

SEQ ID NO: 160 - DNA, 401 bp, Homo sapiens
tgctatagcccgtgccctcatggcctgcctctccctgtcttattcatcttagacacttctgcttttccagcctaagctaacacactgaattagccctgccattcagcaccatccaaggcaaaagggagaaagagggcgtcgccagcatcccagccccctctagtcagtatgcttcaaccacctcttgagaaaggacccttktgggccaccctggaaggaatattgtcctgagcactccctgcaaagccagaagtacagcagagagaaagggctgaccagaaccaatgggtgaaaaccagagggcaggctatttctactaactcacaagcattgagtgtttactctgtgccaagcatttcccaataactagatctgtcagaaaaaggaatggactcttagcc

SEQ ID NO: 161 - DNA, 401 bp, Homo sapiens
taggtcaccagactccgcactgctccaaggacttggcctataccctccatccagctgttgatgggaatcagtacaacacactggagaagaattgggagtaggggctgaggagacaggttctaacttgggctttgctactaacttagtctgtgacctcgagcaatttacgtttttgctcgtgtttcagttacctgtacgttrgatagcagttttaaaaaaaaaaaaaaaaaaaaaaaaaaaaaagctacatgggggctcatgtttgtaatctcagcactttgggaggctgaggtgtgaagattgcttcagcccaggagttcaagactagcctggccacagagtgagaccccatctctacaaaaagaaaaaaaatagccacacatggtggcatgcacctgtag

SEQ ID NO: 162 - DNA, 401 bp, Homo sapiens
ccaggtcactcctgaaaaaaaaatgacctcagctcaaaaattttaaatgaacattgcttagagcctgctgtgtggcaggtgctttccaatacatcagcaaatttcatcactacatcaaacccttccgtggcttcccttcaccaagagtgaaatccaagttcttagcttaatatacaagggtccttgtgatcttggccctgyctgcactttggcctcatctccttctaatacaaatcacagccctcgcttcatcttttcattctccaagaagtctgccctgagcctcctctcaccctctgtctgggttgaggctccttttctgtattcctctaacacccggcagttatccttacttgcagtactttctgtactaccttaccctggtctgtttactcatctgt

SEQ ID NO: 163 - DNA, 401 bp, Homo sapiens
cttattggaagtcatccacttgtttaaaaggatgatgcatactctgtgcataatgtttgataacgaattaattgaagtggaatagcatgagcttacagtttgcagtggaccccgaagccaggctttcattgctaaaggagctaatacttgtttctgtggtttgggtttcctcacaagcagactctgaaacaaggttttgartgcaagtattatagtttatttgggaggtgatcccaggaagtatggtgagggcatatactcaataacggatgccctaatgagcagattatcactgtgagagattgggctccctgcctgtgggcacctccctgacagactgtagaacatgcctcattgtttaactgagaagcaactccttgtctttcactagttgagagttg

SEQ ID NO: 164 - DNA, 401 bp, Homo sapiens
caaagtgctgggattacaggcatgagccaccacacccggctaatatctgtttttaggtgctgagcacaaggagattaagtaatttcccaagaccacacaactagtaagaggaatgaggactcagacctaagtctcctggcaccaaagcacttgacctctagtgatgttcagtgtatctccattttcagacaaggaagttcrtgagactgtatccataaggcaaggtgaggcaactaacaagtagtatgagctcaccacatgccaaaccattcctggtgcttcctctatgccttcctattcctcatgacaatcctatgaggcagatgccatacgtattttactgctggggaagctcaaagcacataattgacataattaacttacctgaagtcaccagctta

SEQ ID NO: 165 - DNA, 401 bp, Homo sapiens
tttgctactaacttagtctgtgacctcgagcaatttacgtttttgctcgtgtttcagttacctgtacgttagatagcagttttaaaaaaaaaaaaaaaaaaaaaaaaaaaaaagctacatgggggctcatgtttgtaatctcagcactttgggaggctgaggtgtgaagattgcttcagcccaggagttcaagactagccyggccacagagtgagaccccatctctacaaaaagaaaaaaaatagccacacatggtggcatgcacctgtagttccagctacttgggaggcggaggtaggaggatcacttgagccccggtggtggaggctgaggtacgctatgattctgccactgcaatctagcttgggcaacagagcaaggccctgcctcacaaaaaaaga

SEQ ID NO: 166 - DNA, 401 bp, Homo sapiens
agttccagctacttgggaggcggaggtaggaggatcacttgagccccggtggtggaggctgaggtacgctatgattctgccactgcaatctagcttgggcaacagagcaaggccctgcctcacaaaaaaagaaaaaaaaaaaaaaggttattttcctacctctcaggattatcagaatcaaatgaaatcaagtatctgagytgactttgcaaagttgatatagggatttgttaactacaaactaaatgtgcccttgatggaaaaggaaccttatggaatgatgacagttaacatttgtagagagcttactctgtgtccagcattgctctaaaagctttccatgaattaactggctcacctgagacatacaacaatctttggaagctaatgttcttaacatc

SEQ ID NO: 167 - DNA, 401 bp, Homo sapiens
gggagcctggagaaagcaaaacaggggttggtcttgcaggaaataggacatggacaggtcatcaaattgaactattaaggacaagggggaagtcaaggactcagatacaagaactggaagataagaggtgggcaagacagaagattccaagacaggaatgatttctaaaggctgaaggagttattgagccaggaggaaagktaatggatggaaaggaaaccaaatccccctgttaaagagaacagagtgcagggagcctggttgtccataacactgtgcattttcaacagtatcctggtcagccgtcatccccaccacactgaaatggacctcctgcaggcaagactgacagggccatcacttggcagaagctaaaataagtaggtttattcccacattta

SEQ ID NO: 168 - DNA, 401 bp, Homo sapiens
ctctttttatcactttcttttcaaaaagaacaagaagagattcactcaccacattcacatttagcaagacacgctctggagtcacattgcttgaacttaaatcccagctgtgccacttactacctacctgacttctaccagtcctatcctcttgtaagcctcagctttctcatctacaaaatggacatgaaaataatagtractacctcacagggtgaatgtccattgctactgtctttggggttgttattattatttatggcatagcaatcatgaacacagacagtagagccagactgtcaggttcaaattctgtctccatttctcagtggctctgtgaccttggcaaagttgcttacacctcagtgtcctcatctgtaagatgaagataatagcctgac

SEQ ID NO: 169 - DNA, 401 bp, Homo sapiens
cagaagattccaagacaggaatgatttctaaaggctgaaggagttattgagccaggaggaaagttaatggatggaaaggaaaccaaatccccctgttaaagagaacagagtgcagggagcctggttgtccataacactgtgcattttcaacagtatcctggtcagccgtcatccccaccacactgaaatggacctcctgcrggcaagactgacagggccatcacttggcagaagctaaaataagtaggtttattcccacatttacccttaaagctttctccctctttttatcactttcttttcaaaaagaacaagaagagattcactcaccacattcacatttagcaagacacgctctggagtcacattgcttgaacttaaatcccagctgtgccacttac

SEQ ID NO: 170 - DNA, 401 bp, Homo sapiens
tttagaatgtactgtataggtgatttgtgggggtaacaaacctaaataatttaaagtagtctttatttgctgagaactgcaggtttttttaaagtatattttaaatctttaaactttcagagattaagagagattggccagggatttatttggagcaggaatttctttttcttgtgcttgcgtctttcccagcatccattctttttgtgcctccatctagaatcatgtaatgtcagcgctagaagagaccaaagacagccatcctttacagcagtagttttcagatttcttttacagccaaatcctttatgcaaaaaaaaaaaaaaaaaaaagtgccactagcaataaaacagggaaaaccagagttacagctgtcctggttggggcttctttgtcccctc

SEQ ID NO: 171 - DNA, 401 bp, Homo sapiens
gtgttcacaactgtcatgccactccatccattgtgctgtaattgctgatttgtttcctcccagtggatcatgagttcttttttgaaagggactgattcttgtgggtctctgtatcctcagcatctaacacagtacctggcacatgataagtgttctcttggctccatacatgtgcatcaaatgagtgaagatataaaagcwggtgttccgtcaacatggcaggtttgacagcaagccacatgcacaggcctggaggtctgagccaaacctccagcacttgggagcctggagaaagcaaaacaggggttggtcttgcaggaaataggacatggacaggtcatcaaattgaactattaaggacaagggggaagtcaaggactcagatacaagaactggaagat

SEQ ID NO: 172 - DNA, 401 bp, Homo sapiens
atacatgtgcatcaaatgagtgaagatataaaagcaggtgttccgtcaacatggcaggtttgacagcaagccacatgcacaggcctggaggtctgagccaaacctccagcacttgggagcctggagaaagcaaaacaggggttggtcttgcaggaaataggacatggacaggtcatcaaattgaactattaaggacaaggsggaagtcaaggactcagatacaagaactggaagataagaggtgggcaagacagaagattccaagacaggaatgatttctaaaggctgaaggagttattgagccaggaggaaagttaatggatggaaaggaaaccaaatccccctgttaaagagaacagagtgcagggagcctggttgtccataacactgtgcattttcaa

SEQ ID NO: 173 - DNA, 401 bp, Homo sapiens
ttattctgtgcaatgaagtcattgcctggttttgagcaagagagtgttgtaacctgatttatgtttgaacagtattgttttggcttctgtgtagtgaaagaattgcaggagacaagagtggagctaacggcagtggcccaggtgagagatgatggcaggaaagtccttggaccaggggcaagtggaggtggaaggaggtgracagctgtgtgatttttttttataaagagttcacaaaatgtacatataattcaaagtagtggatgaggaagagagagaaatcagagacactaatagatctgggacctgagcaactgagtggatgttggtgccacttccttgccttcctagaggaggctcgggatttggagagcaacatgttaggcttggtggagagaagc

SEQ ID NO: 174 - DNA, 401 bp, Homo sapiens
aagagacaacattcttaagcacattaactgagcttgattcatcaacaataattttaagaaaatcctgttggctctgtattccaaacatgtctacaccttgcctacttctcaccagctctaccactcccatccaggcccaatccagcattatctttcacctggattattggaatagtactcaaatcagtctttctttccacwtatgactcctccataatccagtttcttttaaatgtgtcaggcgattcttctgatcaaaacccttcaatggttccccatttcacagggataaaagcccaatctcaagatcacctacgagacaaccccacagtacctggagcccgttcctgccctgatctcatctcccactgctctgccccttgcccagtctcctccagcca

SEQ ID NO: 175 - DNA, 401 bp, Homo sapiens
atgtggctggctgctctaggcactaagactatgaccgaaactcatggtgcatatatttaacaaaggagagagacatagtccattaaacatgtaagatgctggtgatagctcctacaaatataaactaaacaggataagaagggatagagaatgacagaggtgggagtagggagaaagggtaactgttagatagagtcaggraaggccatactcaggaagtcacatttctacagaagcccaaatgtagagagggaatgagccatgccaagttttcaggagaagaggtggttagaaaatattttagcagaagcaacagaaaatgcaaagagtctgagaaaaaatcctcttgcaatttaagaagcattcaaagaatattaagggtgagcaggggaagactaaaa

SEQ ID NO: 176 - DNA, 401 bp, Homo sapiens
ctaggtttgtgtaaatacactctatgatgttggcacaacaaaggtgcctaaggacgcctttctcagaacgtatccctgtcgttaagcaatggatgactgtagttaaaatatgttacgaaatgaaatttgcatctaacactgccacaacaaaacaatttagttccataaaaacacagcttgaaaaccaatggccgactcctrgacttccccaatggatcatatgcaaggtaattttaggtggtatacattcttttttgttaataatggtatattttttaaatgtgcattaaatattgactttcacatttgctatcatgatatacattttcttttactattaaatgacttataaaattaagaaaggtgagttaattttttggaacttttatttttagatacag

SEQ ID NO: 177 - DNA, 401 bp, Homo sapiens
ctatgtattgcttgtgactatatttaaatacagtaaaagtacattggcaatggttatatcctgactcacctctggagagggaaaagggaaaagggccttagcaaatatcaacatctcttaaagctgaatgatggctatataagtttctggtaaatattttccatacttttctggaaagtttaaaagagttcaaaattaaaytggaagattcatatcagggagaaatggggaggagtgggccctccaggctgagggaacatcaccagcagcacagaattgtaaaagcaccccatctgaggagtggccgatagcgtcactttgacccgggatgcctgggtgcgaaagagaatagaaaaggctaagtaggaagcagctgtaccaaggtctctggaccttattct

SEQ ID NO: 178 - DNA, 401 bp, Homo sapiens
gtttgctaactcaagcaaggggaaaggtctgttttcaattaggacctctcaaaaaagtgtttttaaatggcctgtaaaaataatccaataaaggcctcagaggtgtgagctgttgtcatcatcctgtttggaaaacgtcactcagaatgcaccctaaagtgagggagggaggttgtgatgccttaaaaaaaattaatttgygaaatgaacctgcaggattagttgtccgcctttgtgcccagctttaatatgtcctcaaccagggaaatccgaggctttggattattagccgggttggatcacgttaccctgtttggctctgcagaaatcacacttttcattttgcctttaatcctaaaggtcaccgggaaggtcagccccaaacacaaatgatactttgt

SEQ ID NO: 179 - DNA, 401 bp, Homo sapiens
tctgtatgaatttcttcttatttaataccactttgggagtatatatccccactaccttcgaggctgggtcattgccctacttaagaatttacttactagtggctttttaaaatgagagggtagtggtataaaaataatccaatagaatgttatctggaagccaaaaaataagtaaataaataaaaccatagttttgctatktggagtcgagatttaaggtagaggaaagttaaatggctctaagaggcaaagagctgttttctaatgaaggtgttggctcccatcctagtttgcctttttttttttaggactccctgctagccctcaaaaacctgccttgagggcaaactcttcacagttcaaagttttgccctcggaagtaaggctgcattttaatagca

SEQ ID NO: 180 - DNA, 401 bp, Homo sapiens
catttctgcctgctcctttaattcctcttggaaagtttacggttaatattttccctggaacattgtcaagcttttgacagtgcctgagtgtatgccgaactgtgaaattgagccggagaagcaagttgtgagaaatctgtttctactcagatccgtaaggtttatgggggggggaaaaaaaaccaaaaaaaaaaaaaaaamcccaaaaaaacaaaacaaaacaaaaaacaaaaaacttcagaggggaaactgagaatgggactcggcttgcttctcctggtgtgggttcaggccgccattttaaggagccagtgaagggcgacgttccgctccttacatggcggctgtatttactcggccgcagccaatcagccggcagtgccaagccacgtgacatgcca

SEQ ID NO: 181 - DNA, 402 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 182 - DNA, 403 bp, Homo sapiens
cctggccaacatggtgaaaccccatctctactaaaaatacaaaaaatgagccaggcatggtggcaggcgcctgtgatcccagctactcaggaggttgagacaggagaatcacttgaacctgggaggtggaggttgcagtgagctgaggccgcaccactgcactccagcctgggcaacagagtgagactctgtctcaaagaacaaaaaaaaaaaaaagaaaagaaaaaaaagaaatattgtctggctaaagaaaggaaaagaattcttattcagaatcagcatgatcacttctgggaatctgaatggagaaaataattctatatgtaatgatgttttcaatcaatattattttaggtggtttatttattcagccaactgttgttggctgcccactgtataccag

SEQ ID NO: 183 - DNA, 401 bp, Homo sapiens
cgtgttcccagagatgttgggcctggtttagccagttgttaaactgaagcagggttagcttacccttgaagtgtttttgtttgtttgtttttgttgagacagagtctcgctctgtcacccaggctggagtgtagtggtgcgatctcggctcactgcaccctccgcctcccgggttcaagtgattctcctgcctcagccccsgagtaactgggaccacaggtgcgcgcccccacgccagctaatttttgtatttttggtagagatgggatttcgctgtgttggccagcctggtagttttcttacatgaccatctttagatttcagagaaggaagaacatgatcccagaaagcacacagagttacaacatagcaatagcccctccgagctcaacaaaaacatc

SEQ ID NO: 184 - DNA, 401 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 185 - DNA, 401 bp, Homo sapiens
ctaaaggtccatcagcctttagaagaagccacagtggttttcatttctttcactctgtttatcttctgaccaaaggctgactcttccacaggcggctgatatcgagtcaatcaggactctttatgcattatgtgaatttggccctcacacagctgagaatggcctgaatagctaagagagcatcctctctgcagcacctcytgactcctcaggacgagtcacactgaaagaggcaggtggatgcccctgattgtgtcccctcccatccagcatcatggccagcactgccattccttcaccccacccaccccagttagcccctggcttgaactccgtcactcaagagggaacattaaaaaccccacacctctgactcatactttgattttgtggcctaaaga

SEQ ID NO: 186 - DNA, 401 bp, Homo sapiens
tgtcagagaacagtctcagaaagatctgttcctttctttctagactcagtaccacagactggcctatcctctgcaactttgcttagcagcaggagtagagaagtattgattgcccacaacttgcctttaagtcttgtttctgtggtgcaggatttttaaaaagcatttaatgttttccctgccttgaagacttcagaaccktataaatgccactgtttaaagtcctgtccctgctgaaaaccagggcaggtctcatcacagccccatctccattttccttttgttgaagtgggtctgtgtgagagcgggctgtgccctccttctccacagggtggggaaaaggcagccctgtagtaaggaggttgaatagcctcgctcactttgcctcctgcttgaggtgg

SEQ ID NO: 187 - DNA, 401 bp, Homo sapiens
ctttccatgaattaactggctcacctgagacatacaacaatctttggaagctaatgttcttaacatctggtttttgtttttttgtttgtttggttggttgttttttttgagacagagtcttgctctgtcaccaggctggaatgcagtggcgcgatctcggctcactgcaacctctgccccctgggttcaactgattctccygcctcagcctcccgagtagctgggacaacaggcgtgagccaccatgcctggctaatttttgtatttttagtagagacggggtttcaccatgttggccaggctgatctcgatctcttgccctcgtgatctgccagcctcggcctcccaaagtgctgggattacaggcatgagccaccacacccggctaatatctgttttta

SEQ ID NO: 188 - DNA, 412 bp, Homo sapiens
attttattacctatactcataagaattgtattataaaatacattgttaaacgaatgttttcagtgctccattgagagtcggtggagcacactggttgggagaagacagagctgtgagccatccgtctgcctgtgcttgagtcttggctctgccattgactagttgtatgaactgccgcaggtggttcagccactcagaacctcagtatctcatctgtaaaagtgagatgtaaaaacactttctacatcataggattattgtgaagattaaatgtgatatgttgtaaaattctggtcacacaagtattaacttactgttatttttgctgccactgctattaattaatggcagtgtggcggctcagtactaggcaatgggcgtgcaactgtgatgagaaacgcttctgtccatt

SEQ ID NO: 189 - DNA, 407 bp, Homo sapiens
attttattacctatactcataagaattgtattataaaatacattgttaaacgaatgttttcagtgctccattgagagtcggtggagcacactggttgggagaagacagagctgtgagccatccgtctgcctgtgcttgagtcttggctctgccattgactagttgtatgaactgccgcaggtggttcagccactcagaacctcagtatctgtaaaagtgagatgtaaaaacactttctacatcataggattattgtgaagattaaatgtgatatgttgtaaaattctggtcacacaagtattaacttactgttatttttgctgccactgctattaattaatggcagtgtggcggctcagtactaggcaatgggcgtgcaactgtgatgagaaacgcttctgtccatt

SEQ ID NO: 190 - DNA, 401 bp, Homo sapiens
tgagaaactggtggaccgacacactctaattttttggcttctgaccaaacaagctagaaggatgccaaaattcaacaaaataacacattattgtgtgataggagccgtgctccaagagagcaggaactcagaggaacttcatactggccccttttaaaaaagcattgtcactttggggagctttcttagagaaacgagaggaaatggtaaaatgcaacctggagagtaaggtataatttgcacatgaacacgaaggaaggaactgaaagaaaacagaggagtttaaagttacttctatgaacttttcccagacataacacacagttctctgacttgacttacattcttttaaccctgaaagttccatctctgtgtctgagcagaatgctggactgcttaac

SEQ ID NO: 191 - DNA, 401 bp, Homo sapiens
actgtatttccaatagccccctatcgagtgcagagatgttttggggggtgaaagttgattttgattggatttacaaccccatatatcaagcactagactaaaggtccatcagcctttagaagaagccacagtggttttcatttctttcactctgtttatcttctgaccaaaggctgactcttccacaggcggctgatatcragtcaatcaggactctttatgcattatgtgaatttggccctcacacagctgagaatggcctgaatagctaagagagcatcctctctgcagcacctcttgactcctcaggacgagtcacactgaaagaggcaggtggatgcccctgattgtgtcccctcccatccagcatcatggccagcactgccattccttcaccccac

SEQ ID NO: 192 - DNA, 401 bp, Homo sapiens
aaatgcaatgccattcgtaaaagcgtttgtaaatgataaagcgacatgccacctttgagtcattgccattggagctcctgattgaggaatctggtggagtactaggtgctagcagagctttgagggcagctgtttgctttacaagacacacctgaaggctgctatcttggctcaagaagcctgtcctcaaatatcctgacrctttgaaagtcaagggtaagggattcaaaccctatgtaggcccctttctctcatctggtttctggcctactgagagttgctaaccctgctttgcaccaggtgagactgtatttccaatagccccctatcgagtgcagagatgttttggggggtgaaagttgattttgattggatttacaaccccatatatcaagcactag

SEQ ID NO: 193 - DNA, 401 bp, Homo sapiens
aacaaggttttgagtgcaagtattatagtttatttgggaggtgatcccaggaagtatggtgagggcatatactcaataacggatgccctaatgagcagattatcactgtgagagattgggctccctgcctgtgggcacctccctgacagactgtagaacatgcctcattgtttaactgagaagcaactccttgtctttcaytagttgagagttgctcctgagtgcattaagtcccctgccctttcagcctgccccactttgccatgtggacagagaaagccctgaggcagagagactacggtgtttgtgcttcaagttggacagcatgtctgccccagctccaggtgacctccacggagtgtgagcagcatgtggggaggacaccaatagtttctgttaca

SEQ ID NO: 194 - DNA, 403 bp, Homo sapiens
tttctgcctgctcctttaattcctcttggaaagtttacggttaatattttccctggaacattgtcaagcttttgacagtgcctgagtgtatgccgaactgtgaaattgagccggagaagcaagttgtgagaaatctgtttctactcagatccgtaaggtttatgggggggggaaaaaaaaccaaaaaaaaaaaaaaaaaccaaaaaaaaacaaaacaaaacaaaaaacaaaaaacttcagaggggaaactgagaatgggactcggcttgcttctcctggtgtgggttcaggccgccattttaaggagccagtgaagggcgacgttccgctccttacatggcggctgtatttactcggccgcagccaatcagccggcagtgccaagccacgtgacatgccacga

SEQ ID NO: 195 - DNA, 401 bp, Homo sapiens
gctgctatcttggctcaagaagcctgtcctcaaatatcctgacgctttgaaagtcaagggtaagggattcaaaccctatgtaggcccctttctctcatctggtttctggcctactgagagttgctaaccctgctttgcaccaggtgagactgtatttccaatagccccctatcgagtgcagagatgttttggggggtgaargttgattttgattggatttacaaccccatatatcaagcactagactaaaggtccatcagcctttagaagaagccacagtggttttcatttctttcactctgtttatcttctgaccaaaggctgactcttccacaggcggctgatatcgagtcaatcaggactctttatgcattatgtgaatttggccctcacacagctga

SEQ ID NO: 196 - DNA, 401 bp, Homo sapiens
tccatattagtgataaggatgaatttttactaagtgcctaggctatatgctaggtgcttttccaaatgccttaaaaaacaattctaccacgatataggagccattatcctcctttcatataggaggagctgatcatcactgatgttaaataacttgctgaaggatatgtgtaaccagataggtggaatcaggattcaatcygtgtatctccagtgtttgcccctgtgactgtggtaaggctgcagccttattggaagtcatccacttgtttaaaaggatgatgcatactctgtgcataatgtttgataacgaattaattgaagtggaatagcatgagcttacagtttgcagtggaccccgaagccaggctttcattgctaaaggagctaatacttgtttct

SEQ ID NO: 197 - DNA, 407 bp, Homo sapiens
ggcatgcagtgaggagcacctttgtagctagaacatgcttagattttggtattcttgaaaatgtggcctcctccccaatgccagtgtataggatttaaaaaaaaacaaaaaaacacatctcaaaccttggcatttattgaatattaacaggccaggcaccaaagcattattcagcattgacacttaaacttttctgtattgattattattattattattattattattttttgagacaggatctcactctcttgcccaggctggagtacagtgacataatcttggctcactacaacttgtgcctcccaggctcaagtgattctcttgcctcagtcttttgagtagctgggactacaagctcgcaccaccacacccatctaatttttgtattttttgtagagacgggc

SEQ ID NO: 198 - DNA, 415 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 199 - DNA, 401 bp, Homo sapiens
agcactggtgaatacgactttattaccaagagagaagagtagcttttagttgtggcttagctggaatatattgatgataagaatggctcagcagacagaagtcttgggactctcaaaaaggctccaagtgtgctttcttttaaaaaagttatttaggcccatcctttataaacacccaagtagatggtctgatggggtcayggtaacaaagattcagcttctatctaggtggatggtaagacccgctaacatcttggcaaaccgtgttattgggccattaaggaccagtgcttgaattctggggctgaaaattcaacgtattcccttataagaaaatgtctgctcatgataagaagtcacacaaagtacaacctcactatagtacaggatttagaatcttt

SEQ ID NO: 200 - DNA, 401 bp, Homo sapiens
ttaaagtagtctttatttgctgagaactgcaggtttttttaaagtatattttaaatctttaaactttcagagattaagagagattggccagggatttatttggagcaggaatttctttttcttgtgcttgcgtctttcccagcatccattcttttttgtgcctccatctagaatcatgtaatgtcagcgctagaagagacyaaagacagccatcctttacagcagtagttttcagatttcttttacagccaaatcctttatgcaaaaaaaaaaaaaaaaaaaagtgccactagcaataaaacagggaaaaccagagttacagctgtcctggttggggcttctttgtcccctcacctctcttcactctcatgcaaatgcctcacagaacccctgaagaacac

SEQ ID NO: 201 - DNA, 401 bp, Homo sapiens
tagagttctcaagagatctggtagttcaaaagtgtgtggcaccttcccctcccctctctctctccctctctgccatgtgaagaaggtgctcacttacactttgccttctgccatgagtgtaagtttcctgaggcctccccagccatgcttcctgtacagcctttggaactgtgagtcaattaaaccttttcttcataaattaaaaaaaagaaagaaagaaaatttaatgacagtctaggctccccattagtgagacatgtcctcagtgaagtaagtgcaacttgtaacaacaataattcatcttcctagactccataaaggaaagaacattgcttttagcttggttttgaccttcacctttagggaccaccactaccatcagcccctgccatcattatgcc

SEQ ID NO: 202 - DNA, 401 bp, Homo sapiens; identical to SEQ ID NO: 45

SEQ ID NO: 203 - DNA, 407 bp, Homo sapiens
tgattttactgggttatttatttatttttagagacagggtcttgctctacaacccaggccggatttcagtgatgcatccatagctcattgtaacctcaaactcctgagtttaagtgatcctcctgcctcagaacctaagcacctgggactacaggcatgtgccaccataccaggctaatatatatatatatatatatttttttaattttttttatttttttattttttgtagagactgtgtctttcctacgttgctcaggctgctcttgaactctaccctccgaaagtactgggattacaggcatgatccacaggacccagccctagatcttctatttttgattgtgaaataacctctgattgtgaagacacctgctttaagagcttttttcccaaaagaattgtga

SEQ ID NO: 204 - DNA, 401 bp, Homo sapiens
gcaggggttgcaatcctactgataaaacagactttaaaccaacaaagatcaaaaaagagaaagaaggacattccataatggtaaagggatcaatgcaacaagagctaactatcctaaatatatatgcacccaatacaggagaacccagattcataaagcaagttcttggagacctacaaagagacttggactcccacacartaatagtgagagactaacaccccactgtcaatattagatcaatgagacagaaaattaacaaggatatccaggacttgaactcaactctggaccaagcagacctaatagacatctacagaactctccaccccaaatcaacagaatatacattcttctcagcaccgcatcacacttattctaaaactgaccacataattgga

SEQ ID NO: 205 - DNA, 401 bp, Homo sapiens
caaaacaacaacaaatatacatatacacttacatttccctaaagaaatattgagataatatacaaaaactaataaaaggatttaccaaaagggagatgggaaatggagtggacagagatgcagctatgagctatgagcaagttttctcaatgagtatatttatatcattttcatttttgaacagtattgtctattcaaaataaaattctgccacagattagggggaaaataagaatagtctctttgatggggatggccatgtgcatatctctcagaaatcccacatggggagcaggaggctaggacttccaggtggcatagcattttcaacacaagtcacgttcatcacaaggtgggggaatcatcagagggttcctttgatggatgggatgtggaggtgg

SEQ ID NO: 206 - DNA, 418 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaattgtgtgtgtgtgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 207 - DNA, 401 bp, Homo sapiens
gtccttgcgatggtttgctgagaatgatggtttccagtttcatccatgtccctacaaaggacatgaactcatccttttttatggctgcatagtattccatggtgtatatgtgccacatttttttaatccagtctatcgttgttggacatttgggttggttccaagtctttgctattgtgaatagtgccacaataaacatgygtgtgcatgtgtctttatagcagcatgatttatagtcctttgggtatatacccagtaatgggatggctgggtcaagtggtatttctagttctagatccctgaggaatcaccacactgacttccacaagggttgaactagtttacagtcccaccaacagtgtaaaagtgttcctatttctccacatcctctccagcacctg

SEQ ID NO: 208 - DNA, 401 bp, Homo sapiens
ccctggaggctgaagacccactcacttcatcccatcctcatcgccaattgtcagatcccagggtctgggtccagcccatgctgaagtctgagggagtaggtggatgggcagcaagaacacttagggggctgtagggaggtgaaatgtagttttatccagaagctgtctcatcaacagttttctcaccgtccgccctgtctyggctgcttgagctggccacctccatgcacagctgtgtggccagctccccctggccttcagggtcagcagcttaactctttctctctgtgcatgagcgagtcgagctgtgtcctggctcccttctgtctgtctgcaaagatggacagctctggctcactctctctctgggcatcagcaggcctaccatgttaagccatatt

SEQ ID NO: 209 - DNA, 401 bp, Homo sapiens
taggtgactgattaaattacggtaaagccgtacaaaaaatacaaagatacggtaaagccgtatcatggtcatttgtaccagtaccaggttaactgggtaaacttagaagtacatttcccagaacccctttcctgtaggattctaagtgataattggccaaaagaggaattagtgggacatttgaaaggtggaagtgaacartacctgtttctctcttaaaggcatggagattggatgggagacagactcagagaggctggtggttgctggcaagccctcactcttctttgccccatgttcaacttctcctcctgactggcaccttgctagagacccacagaggaaacagcctcccatagattccccccagcttcccctttgttgatccgctttgtggcctg

SEQ ID NO: 210 - DNA, 401 bp, Homo sapiens
tcatgcctgtaacctcagtgctttggaaggctgaggtggaagggtccctggatcccaggagccccaggctgcagtgagctatgattggccatagcactccagcctggacaacaaggtaagaccctgtctctaaataaataaacaaataaaacccagaagaacaaaatggattgtttctaagtgcaaatattctactttatyggttgggcatggtggctcatagctgtaatcccagcacttttggaggccgaggcaggtgaattgtttgaggtcaagagttcaagaccagcctggccaacatggcaaaactccatctctactaaaaatacaaaaattagccaggtgtggtggtatgtgcctgtaatctcagctacttgggaggctgatgcaggagaattgct

SEQ ID NO: 211 - DNA, 402 bp, Homo sapiens
attctgtgtcacgctctgtgtaagtgactccatgatcaaagctttcctgttgtaattgtgtggatttacttgttgcccactgtccccataccctccacccccacatggtgtgctttctccagaaagggactacttctgctgaccacacagaagacgtgtgtaaagtctgtgtatcaatgaatggattctcatctttcatagttttttttttaaatagttttatgtgtgtttaacttaatttcacttaaaaagatatttaccagaagctgaaagtagggtgtgatgaggttgggttcaggaaggactggtatcacatggcttccctaagttgtatattacattgttaggacacctgacagagctgtggattagtgaatcttacggatggctcttttcagttga

SEQ ID NO: 212 - DNA, 401 bp, Homo sapiens
cacttgaattggaatgtctttagatggaatctgtgccttctagtttgccataatccccactgttccctattatattatgttgtatcagcagcctgcttctatcatttgcctgcagagtctataagcatttatgattccttgtaattattgatcatgtggtcttttgttgctatactaagggtctaaatctgattcaggttagcctttgactgtaactgtaattctctaactttcaacccttttatcatcaaggacctcaactattattttttgttccatatttgaaaacttttggtgttccagacacactgcattggttaataactaattttcccgttgtaaaaacagacacgtgtaactgaacacacaaatgagccatcaacagtatgaatataaaagtg

SEQ ID NO: 213 - DNA, 410 bp, Homo sapiens
cacttgaattggaatgtctttagatggaatctgtgccttctagtttgccataatccccactgttccctattatattatgttgtatcagcagcctgcttctatcatttgcctgcagagtctataagcatttatgattccttgtaattattgatcatgtggtcttttgttgctatactaagggtctaaatctgattcaggttagctgttgatgcctttgactgtaactgtaattctctaactttcaacccttttatcatcaaggacctcaactattattttttgttccatatttgaaaacttttggtgttccagacacactgcattggttaataactaattttcccgttgtaaaaacagacacgtgtaactgaacacacaaatgagccatcaacagtatgaatataaaagtg

SEQ ID NO: 214 - DNA, 401 bp, Homo sapiens
ctagacacaagtctgatttttcattccagagcagcaaataaagtcatagtggacagctgcttcagtctggaaactagaaacaaacaagaggtgttagctggcagctgaacaatgaagaaagacatggagacactgtccaagaggtcgagatggatagtagcttgagatcctctctttctctctagacatgcgccatgtgcaacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacccctctataatcctgtatacttaaattcctaaatagtt

SEQ ID NO: 215 - DNA, 405 bp, Homo sapiens
ctagacacaagtctgatttttcattccagagcagcaaataaagtcatagtggacagctgcttcagtctggaaactagaaacaaacaagaggtgttagctggcagctgaacaatgaagaaagacatggagacactgtccaagaggtcgagatggatagtagcttgagatcctctctttctctctagacatgcgccatgtgcaacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacccctctataatcctgtatacttaaattcctaaatagtt

SEQ ID NO: 216 - DNA, 407 bp, Homo sapiens
ctagacacaagtctgatttttcattccagagcagcaaataaagtcatagtggacagctgcttcagtctggaaactagaaacaaacaagaggtgttagctggcagctgaacaatgaagaaagacatggagacactgtccaagaggtcgagatggatagtagcttgagatcctctctttctctctagacatgcgccatgtgcaacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacccctctataatcctgtatacttaaattcctaaatagtt

SEQ ID NO: 217 - DNA, 409 bp, Homo sapiens
ctagacacaagtctgatttttcattccagagcagcaaataaagtcatagtggacagctgcttcagtctggaaactagaaacaaacaagaggtgttagctggcagctgaacaatgaagaaagacatggagacactgtccaagaggtcgagatggatagtagcttgagatcctctctttctctctagacatgcgccatgtgcaacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacccctctataatcctgtatacttaaattcctaaatagtt

SEQ ID NO: 218 - DNA, 411 bp, Homo sapiens
ctagacacaagtctgatttttcattccagagcagcaaataaagtcatagtggacagctgcttcagtctggaaactagaaacaaacaagaggtgttagctggcagctgaacaatgaagaaagacatggagacactgtccaagaggtcgagatggatagtagcttgagatcctctctttctctctagacatgcgccatgtgcaacacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacccctctataatcctgtatacttaaattcctaaatagtt

SEQ ID NO: 219 - DNA, 421 bp, Homo sapiens
ctagacacaagtctgatttttcattccagagcagcaaataaagtcatagtggacagctgcttcagtctggaaactagaaacaaacaagaggtgttagctggcagctgaacaatgaagaaagacatggagacactgtccaagaggtcgagatggatagtagcttgagatcctctctttctctctagacatgcgccatgtgcaacacacacacacacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacccctctataatcctgtatacttaaattcctaaatagtt

SEQ ID NO: 220 - DNA, 423 bp, Homo sapiens
ctagacacaagtctgatttttcattccagagcagcaaataaagtcatagtggacagctgcttcagtctggaaactagaaacaaacaagaggtgttagctggcagctgaacaatgaagaaagacatggagacactgtccaagaggtcgagatggatagtagcttgagatcctctctttctctctagacatgcgccatgtgcaacacacacacacacacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacccctctataatcctgtatacttaaattcctaaatagtt

SEQ ID NO: 221 - DNA, 401 bp, Homo sapiens
aaccattctctttcttttctttttttcaaaattagagacagggtcttaatttgtcacccaggctggagtgcaatggcacgatcctagctcactacagcctcgaactcctgggcttaagggatcctcctgccccagccgcatgagtagcaagtgcatgccaccatgcctggttaatttctttcttttctttttctttctttctttttttttttttggatgagatatgggtctaattatgttgaccaggctggtctcgaactcctggcctcaagcagtcttctcaccctaggcccccagaatgctgggattacaggctttagcaaccacacccagcctgaaccatttcctttctgatttaacttaggaaagtttgctgcatagtaggagctcagctaacattt

SEQ ID NO: 222 - DNA, 402 bp, Homo sapiens
aaccattctctttcttttctttttttcaaaattagagacagggtcttaatttgtcacccaggctggagtgcaatggcacgatcctagctcactacagcctcgaactcctgggcttaagggatcctcctgccccagccgcatgagtagcaagtgcatgccaccatgcctggttaatttctttcttttctttttctttctttcttttttttttttttggatgagatatgggtctaattatgttgaccaggctggtctcgaactcctggcctcaagcagtcttctcaccctaggcccccagaatgctgggattacaggctttagcaaccacacccagcctgaaccatttcctttctgatttaacttaggaaagtttgctgcatagtaggagctcagctaacattt

SEQ ID NO: 223 - DNA, 403 bp, Homo sapiens
aaccattctctttcttttctttttttcaaaattagagacagggtcttaatttgtcacccaggctggagtgcaatggcacgatcctagctcactacagcctcgaactcctgggcttaagggatcctcctgccccagccgcatgagtagcaagtgcatgccaccatgcctggttaatttctttcttttctttttctttctttctttttttttttttttggatgagatatgggtctaattatgttgaccaggctggtctcgaactcctggcctcaagcagtcttctcaccctaggcccccagaatgctgggattacaggctttagcaaccacacccagcctgaaccatttcctttctgatttaacttaggaaagtttgctgcatagtaggagctcagctaacattt

SEQ ID NO: 224 - DNA, 404 bp, Homo sapiens
tctttctctctagacatgcgccatgtgcaacacacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacacacctctataatcctgtatacttaaattcctaaatagttgtttgggtgttcactatattggaacgctttaacttgtgttcttaataatatctttaggaaaagattaaagcatgtttctgcatataataatattagtaacaaatgatggaagattttgctccaaaatgagttaatgtagaaaacaggtagtgattaaagtggt

SEQ ID NO: 225 - DNA, 403 bp, Homo sapiens
tctttctctctagacatgcgccatgtgcaacacacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacaccccctctataatcctgtatacttaaattcctaaatagttgtttgggtgttcactatattggaacgctttaacttgtgttcttaataatatctttaggaaaagattaaagcatgtttctgcatataataatattagtaacaaatgatggaagattttgctccaaaatgagttaatgtagaaaacaggtagtgattaaagtggt

SEQ ID NO: 226 - DNA, 404 bp, Homo sapiens
tctttctctctagacatgcgccatgtgcaacacacacacacacacacacacacacacacacacgcagacagtctctgactttcaacggtttgactttatgatgagtttatcaggatgtaactctgtcacaagttgaggagcatgtgtttatgtgtgtatgtgtatccgtatacatttacatttatatatacacacacacacccccctctataatcctgtatacttaaattcctaaatagttgtttgggtgttcactatattggaacgctttaacttgtgttcttaataatatctttaggaaaagattaaagcatgtttctgcatataataatattagtaacaaatgatggaagattttgctccaaaatgagttaatgtagaaaacaggtagtgattaaagtggt

SEQ ID NO: 227 - DNA, 404 bp, Homo sapiens; identical to SEQ ID NO: 224

SEQ ID NO: 228 - DNA, 403 bp, Homo sapiens; identical to SEQ ID NO: 225

SEQ ID NO: 229 - DNA, 404 bp, Homo sapiens; identical to SEQ ID NO: 226

SEQ ID NO: 230 - DNA, 411 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgcatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 231 - DNA, 407 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgcatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 232 - DNA, 415 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgcatctatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 233 - DNA, 419 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgcatctatctatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 234 - DNA, 423 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgcatctatctatctatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 235 - DNA, 427 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgcatctatctatctatctatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 236 - DNA, 417 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgcgcatctatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 237 - DNA, 416 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgctatctatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 238 - DNA, 426 bp, Homo sapiens
gaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgctctatctatctatctatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttattttaaaccagacataaacaggacataaaatg

SEQ ID NO: 239 - DNA, 406 bp, Homo sapiens
ttatgcctgtttatacgatcactcgctgtagcagtatacaaaaaattctgttgatctgcattctctccagaatttggcactgccagatttttctttttgccaatcttgaggctaaaaaagagtatttcattgtgtttttaatttgcatttataatttgattactaatgagactaaacatctttttgtatatgtatgagccactttcctttactgtggaataaatgtttttgtcatttattcatttttttctattttattgcttattgtttacttattggtttgtaggagttctttatagattctgcattctaatttttggccagtgtacgtttgccaatatattttcgtagtttctggcttgttttaaaattttcttcatgttatctttgatcaacaaaaattctt

SEQ ID NO: 240 - DNA, 401 bp, Homo sapiens
cttgggttttgatgaaagaattccccaattcagttaaattcatccttgactgtcgtgtgccactcatgttcacttggttaaaaaaaaattgttttttggagattatttgtagaatctcggttctgtaaccagatattgaatattaccactggagggaagctttgaactcatttatcacccttctgccaaaccacaaaaatcctctctctctctctcatgcatctatctatctatctatctatctatctatctatatcttactgatttattcatatatatatttcttcatatatatgatatatgacactgtatatttacagcatatatattacagcacagctatttacagcaacctggatcattcattcttagccccttctcaagaatggaagtttatttta

SEQ ID NO: 241 - DNA, 402 bp, Homo sapiens
gcatgtgatgggtgaatgagtgtttcagtgaaatgacataagtctgtataatttggagggtaatgatgccttagaacaagaataaatctggagcgatggaaaggctccatattctagatgaatgcatgcttcctcttatgactctgaaaaataaaattaaatctttatttatacaaatccagtgaggggggaaggctacatgggtttggcttaatgatatatttcagaacaggaatattagccttaacctctttcctcacattgcatatgatatttaatccatcatctttgttttaaacaaacaatacacaagctgttgctggcattggtataaagctgatggtccatctggagagcaggaatatagatcaggaaaataagagaattgaaattgggtgcaag

SEQ ID NO: 242 - DNA, 407 bp, Homo sapiens
caccattgcactccagcctgggcaacaagagtgaaactccatctcagaaaaaaaaaaaaaaaaaaaagagaatatgtttggtagaaatctgaaagagaatttatgctgaattgagaccatttggaaggcttcttgggtaaaactgatttgagttgtggatgaaggattgtttggaaatgagagaatgagcagaggccatggtggagaggagagaagagttggagagcaggtgaaaggcgtgagcacagctgcagaagcagacatatgcacgatttgtcctagagcaggtcggttggcgagtttggttagaatggggggtttacatagcggagggtattgaatgccaatttaaagatctaggcagtaagaatcatgtagggtttttgaacagggatatgacatgcttc

SEQ ID NO: 243 - DNA, 401 bp, Homo sapiens; identical to SEQ ID NO: 212

SEQ ID NO: 244 - DNA, 410 bp, Homo sapiens; identical to SEQ ID NO: 213

SEQ ID NO: 245 - DNA, 403 bp, Homo sapiens
ttgcttttctctctccaggatccagcacctggcctggcacagggtacatgctcagagaacaagtctttgaaagaatgggtagatgtttattttcctttgtattagccattagctcaaggtctgcagctacttaattccaacctgggtccatttttagcagaagaaaaaagaataatgggactcagcatcaaggcgcacctgacacacagagtcctcttggaaatgtgtgacctgcctcagtttagccactgcttttacttcatcctcatcagtcagagtatgacattgccttcccctttacctcttaattttggaatatttcaagtgcctctaaaattttatttaattaaggggcttccaaatctgcttgtagatattttattcttgaaatgcttgtggcatt

SEQ ID NO: 246 - DNA, 401 bp, Homo sapiens
atttctttaccttggaactttagaagagggtctgaactgagcaaaaattagtgtccctgcctttttaacggctggacacttatcacaaagctgtgccaacatcagtgatggtgcacccacaaaggtgtttggtcttgataagcttctaaagaagcagactttgttgttgttttaaacagtaatgaactgtttcagtttcataaaaaaaagagacattctttcttaaatagaaaagggcagaaagtttatagagaataatgtctaacttgctaatgcagtgtttgcctttgctctgtggcatgtgtgtgtgtgtgtgtttatgtaggcatgcctacacggctgcttgtgttaatacttagtataaagccttaaaatggataccagattggctatgtaacctt

SEQ ID NO: 247 - DNA, 402 bp, Homo sapiens
atttctttaccttggaactttagaagagggtctgaactgagcaaaaattagtgtccctgcctttttaacggctggacacttatcacaaagctgtgccaacatcagtgatggtgcacccacaaaggtgtttggtcttgataagcttctaaagaagcagactttgttgttgttttaaacagtaatgaactgtttcagtttcataaaaaaaaagagacattctttcttaaatagaaaagggcagaaagtttatagagaataatgtctaacttgctaatgcagtgtttgcctttgctctgtggcatgtgtgtgtgtgtgtgtttatgtaggcatgcctacacggctgcttgtgttaatacttagtataaagccttaaaatggataccagattggctatgtaacctt

SEQ ID NO: 248 - DNA, 402 bp, Homo sapiens
tgacattttgcagtttttgttgttgttgtttgtttgtttgtttttttgagacagtctccactccgttgcccaggctggagtccagtggcacgatctcagctcactgcaacctctgcctcccaggttcaagtgattttcattcctcagcctcccaagtagctgggactacaggcttgcaccaccgtgcctggctaatacagctttttttttttttttcttaattttatcataggtaagggaagacgatccaatgtgcagagaaggctcaggttttcattttagtctgcgggtgattgatttctttctttcaaggggctggttgaggaggtcagagtcttagaaagggagaagaaatcagggaaaaggagaaaagaaggaatgagatttatgaccctctggatc

SEQ ID NO: 249 - DNA, 403 bp, Homo sapiens
tgacattttgcagtttttgttgttgttgtttgtttgtttgtttttttgagacagtctccactccgttgcccaggctggagtccagtggcacgatctcagctcactgcaacctctgcctcccaggttcaagtgattttcattcctcagcctcccaagtagctgggactacaggcttgcaccaccgtgcctggctaatacagcttttttttttttttttcttaattttatcataggtaagggaagacgatccaatgtgcagagaaggctcaggttttcattttagtctgcgggtgattgatttctttctttcaaggggctggttgaggaggtcagagtcttagaaagggagaagaaatcagggaaaaggagaaaagaaggaatgagatttatgaccctctggatc

SEQ ID NO: 250 - DNA, 402 bp, Homo sapiens; identical to SEQ ID NO: 247

SEQ ID NO: 251 - DNA, 401 bp, Homo sapiens; identical to SEQ ID NO: 246

SEQ ID NO: 252 - DNA, 401 bp, Homo sapiens
ctggaatttaattatcagtgccataaataatcttgtgaatggaagcagtgtatttggcagtgaatttctgcttcctaaagagaaaggaacctttagaagttatttgaaataattctgtattagccacgatcctggaggcaaatggtcacagaagcagaggatggtatccccagagaaaagtgggttttagatgagtcagatatgtggatatgtgctggtgacgaatgacatgaaggttggatgtattttttaaaatacaaatttaaagcaggctgtatttagaagtttatttataattggttttaggataaagccagcctgttgatgcataacagagttgatcttttggttccattagcacccttgaaatatttaacaagaagctgactttagcatctgag

SEQ ID NO: 253 - DNA, 403 bp, Homo sapiens
tcctggctaacaaggtgaaaccccgtctctactaaaaatacaaaaaattagccgggcgcggtggtgggtgcctgtagtcccagctactcaggaggctgaggcaggaggatggcgtgaacccgggaagcggagcttgcagtgagccgagattgcgccactgcagtccgcagtccggcctgggcgacagagcgagactccatctaaaaaaaaaaaaaaaaaaatctagagttgaaatttttctcttacatttccttttccctctagatcaatcccaattaaagtttcattgcaaaagtttcacaaactcatattggcattaattattattggtgtgctggtgaaagtcctaaagtgagttcattaagaattaaaaaccttggctgggcgcggtggctcacgcctg

SEQ ID NO: 254 - DNA, 405 bp, Homo sapiens
tcctggctaacaaggtgaaaccccgtctctactaaaaatacaaaaaattagccgggcgcggtggtgggtgcctgtagtcccagctactcaggaggctgaggcaggaggatggcgtgaacccgggaagcggagcttgcagtgagccgagattgcgccactgcagtccgcagtccggcctgggcgacagagcgagactccatctcaaaaaaaaaaaaaaaaaaaatctagagttgaaatttttctcttacatttccttttccctctagatcaatcccaattaaagtttcattgcaaaagtttcacaaactcatattggcattaattattattggtgtgctggtgaaagtcctaaagtgagttcattaagaattaaaaaccttggctgggcgcggtggctcacgcctg

SEQ ID NO: 255 - DNA, 406 bp, Homo sapiens
tcctggctaacaaggtgaaaccccgtctctactaaaaatacaaaaaattagccgggcgcggtggtgggtgcctgtagtcccagctactcaggaggctgaggcaggaggatggcgtgaacccgggaagcggagcttgcagtgagccgagattgcgccactgcagtccgcagtccggcctgggcgacagagcgagactccatctcaaaaaaaaaaaaaaaaaaaaatctagagttgaaatttttctcttacatttccttttccctctagatcaatcccaattaaagtttcattgcaaaagtttcacaaactcatattggcattaattattattggtgtgctggtgaaagtcctaaagtgagttcattaagaattaaaaaccttggctgggcgcggtggctcacgcctg

SEQ ID NO: 256 - DNA, 407 bp, Homo sapiens
tcctggctaacaaggtgaaaccccgtctctactaaaaatacaaaaaattagccgggcgcggtggtgggtgcctgtagtcccagctactcaggaggctgaggcaggaggatggcgtgaacccgggaagcggagcttgcagtgagccgagattgcgccactgcagtccgcagtccggcctgggcgacagagcgagactccatctcaaaaaaaaaaaaaaaaaaaaaatctagagttgaaatttttctcttacatttccttttccctctagatcaatcccaattaaagtttcattgcaaaagtttcacaaactcatattggcattaattattattggtgtgctggtgaaagtcctaaagtgagttcattaagaattaaaaaccttggctgggcgcggtggctcacgcctg

SEQ ID NO: 257 - DNA, 408 bp, Homo sapiens
tcctggctaacaaggtgaaaccccgtctctactaaaaatacaaaaaattagccgggcgcggtggtgggtgcctgtagtcccagctactcaggaggctgaggcaggaggatggcgtgaacccgggaagcggagcttgcagtgagccgagattgcgccactgcagtccgcagtccggcctgggcgacagagcgagactccatctcaaaaaaaaaaaaaaaaaaaaaaatctagagttgaaatttttctcttacatttccttttccctctagatcaatcccaattaaagtttcattgcaaaagtttcacaaactcatattggcattaattattattggtgtgctggtgaaagtcctaaagtgagttcattaagaattaaaaaccttggctgggcgcggtggctcacgcctg

SEQ ID NO: 258 - DNA, 409 bp, Homo sapiens
tcctggctaacaaggtgaaaccccgtctctactaaaaatacaaaaaattagccgggcgcggtggtgggtgcctgtagtcccagctactcaggaggctgaggcaggaggatggcgtgaacccgggaagcggagcttgcagtgagccgagattgcgccactgcagtccgcagtccggcctgggcgacagagcgagactccatctcaaaaaaaaaaaaaaaaaaaaaaaatctagagttgaaatttttctcttacatttccttttccctctagatcaatcccaattaaagtttcattgcaaaagtttcacaaactcatattggcattaattattattggtgtgctggtgaaagtcctaaagtgagttcattaagaattaaaaaccttggctgggcgcggtggctcacgcctg

SEQ ID NO: 259 - DNA, 402 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 260 - DNA, 408 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 261 - DNA, 410 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 262 - DNA, 412 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 263 - DNA, 414 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 264 - DNA, 416 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 265 - DNA, 418 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 266 - DNA, 420 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 267 - DNA, 424 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 268 - DNA, 410 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacagacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 269 - DNA, 411 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgacacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 270 - DNA, 410 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgagaacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 271 - DNA, 412 bp, Homo sapiens
taataatgcagattctttaaaaattctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgagaacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagcc

SEQ ID NO: 272 - DNA, 407 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgagtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 273 - DNA, 411 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgagtgtgtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 274 - DNA, 419 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgagtgtgagtgtgtgtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 275 - DNA, 413 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgagtgtgtgtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 276 - DNA, 419 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgagtgtgtgagtgtgtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 277 - DNA, 415 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgagtgtgtgtgtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 278 - DNA, 417 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgagtgtgtgtgtgtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 279 - DNA, 413 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgtgtgtgtgtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 280 - DNA, 419 bp, Homo sapiens
tcgcttgaatccaggaggcagaggttacagtgagcactccagcctgggtgacggtgcaagactctgtctcaaaaacaaaaaacaaaaagaggaataatagtatctgctctccttgccttttgtgggtttttttgatgattaaatgatatctgagggatacaaaaatgctttggaaactacagggtgctttacagactgtgtgtgtgtgtgtgtgtgtgtgtgtgtgtgtgtgtgtgtgtgtgtgtgtgtacagaaaatcctttatctctttgcttctcaatttcttttcttaggaaaatcagattatttaaatcccattatgcacagctctcctctgttcttactaagcctctgtattccattacctccagtaaatcagtaaaaggtggtgagtcaggctgtagtggaaagcggggtct

SEQ ID NO: 281 - DNA, 401 bp, Homo sapiens
gtctttcagaagtgagttagtacagctactttaaataccagttgtgtagattcccacacttttctaccaatggagaggtttacacaagcataatttaagttacaattacactaattaacatctcatttgcataaattgttgaagtcaaaacaacaaagaattgtctaagaagcttaatcctatttgtccaaaatagaaagatttttttttttaaaaaaaaactatgctctaaaaattggcagcttaagtatgtctttagtatgttgagctgtgtccttttaaaaataaatgttttcaattttcttaataatatatttctctattctttttagaatccttcatttttagtatacttttaaaattgaacacacacaacactttggttcctaaagtaatgataa

SEQ ID NO: 282 - DNA, 402 bp, Homo sapiens
tgggtgtgattcaaatcttgtctgtactgctgatgggctgtgtgactttgggcaagtagcttaacttctctgagttcccctgtctctgttttttcatttgtaaaatggagtggaggggacaatattaacttgcaggatggcttgatgatgagaaatgataaatgtcttagtctatattagatcttcagtaaatggtagttgttttgaccactgttactgcaatgagccaaggtggctataagcccttcagtgtttcagtaaggacaagcttacaggtaaccaccaagatcagggcagaacagctgatttaggtctaaacaggttccatcgtgtgtcttcaaaaaggttttcctttttttcctctggagaaaattcagactggtttaagaaggaaactgagag

SEQ ID NO: 283 - DNA, 401 bp, Homo sapiens
aaaacttcagaggggaaactgagaatgggactcggcttgcttctcctggtgtgggttcaggccgccattttaaggagccagtgaagggcgacgttccgctccttacatggcggctgtatttactcggccgcagccaatcagccggcagtgccaagccacgtgacatgccacgagggcacgcacagccatttccttgtttctaaaaaacttgctacctccacagagtactttaccttgttttgcatgccaaatgttcttgctgaatgtgtctagcagactggcatttgtccataaagttattttagtaggtaaaaagtctctgagcacttgagctttgtgcattctttatgtaaaatggatttcccttcttggccagaggccaagggtacagcacactcgct

SEQ ID NO: 284 - DNA, 403 bp, Homo sapiens
ctcgcgggcacccggccgggccggcgcgggagcgggaaagggtgcgctatgcctttaacacccgcgtacagtaggcatgtatagtggagtgtagggaaactctaggcggggttaaagttcagctcatggagcggcaatagcgctggctggctggctgcagttgagccgacttggaaatgtgaacgcaagaagcaggcttgatttttttttctccccccttctctctctctctctctctctctcttcctctctccctctttctcctctctcacccacactcacgcacacctccaaaccgcacacccagacgcacacgcataccccagcgcccggcagttatgtattctccgctctgtctcacccaggtaagccgcggcgtggatgcggagggcttgggggccgg

SEQ ID NO: 285 - DNA, 403 bp, Homo sapiens
tccttctctagagcctcaaacccctgctcagcatgaaaaaaacaacagaaacccaagttaacatctccttgcaatatctgatctgtttttccaatacatctgctcatcttgtttcaaaacaagtagctgtcaccattcttaaccctgtcgtccaaaccagaaaccgggcatcatctttgactggtcccctttactcagggggaaaaaaaaaccatgtcttttaaagtcagcgcctataatactggtctttggtttatctccaataactcgattgttaacagcccttgaaggggaggcaatactgttaaacttgataatttctaaagagttttgagctatttagcacgaagtgatgccaagaaaaaggaatactaacattactcacagcagagggaaaaatttt

SEQ ID NO: 286 - DNA, 402 bp, Homo sapiens
ttttaattattttatgttatacaatttaagtcatggaaagggggatgactgtattgtatcttttaagtataatgtatagcctttaatattcttaaagtggatgttagttaaggacaatttttagttgagagagagtgaaagagagagagataaggggggcagagaggattccattacattcagcacagtatgaaactaagtccaaaggagttttgttaattaaattcaattgccatccattagaccagtggaatgagatgactctgcctggtgctgacacagcacaggtatgcaatttggctaaatggccatttccaaaccatagcacacatttgtctacttgttcacttttttttttttttacatgagagtttttactcttagaaaagtcaaagagtaacc

SEQ ID NO: 287 - DNA, 402 bp, Homo sapiens
acgagaagtccttttcccccctccatctctacagatggcaaatggtaggtcccaactgtcattgttcacaaaaaaggttatggttcaaagtcaaagattcagagataccacaataatcaatcataggaacttgtctcagagtgcccagccaggcaaaagttaggcagagtaataatatttactgagaatctcttatgagtattttttttttggtgtgttctttattttatttagaaaatattatttaattaattgaaatgcctctgaatttagtgacaagcatttaaataaatatgaaaaataatggtcaaaaagttttctgtttatcggttttatcagatagtgctagaatacataattttaaaatgggtgtaacacagaaaataacattcttaatatatt

SEQ ID NO: 288 - DNA, 403 bp, Homo sapiens
acgagaagtccttttcccccctccatctctacagatggcaaatggtaggtcccaactgtcattgttcacaaaaaaggttatggttcaaagtcaaagattcagagataccacaataatcaatcataggaacttgtctcagagtgcccagccaggcaaaagttaggcagagtaataatatttactgagaatctcttatgagtatttttttttttggtgtgttctttattttatttagaaaatattatttaattaattgaaatgcctctgaatttagtgacaagcatttaaataaatatgaaaaataatggtcaaaaagttttctgtttatcggttttatcagatagtgctagaatacataattttaaaatgggtgtaacacagaaaataacattcttaatatatt

SEQ ID NO: 289 - DNA, 402 bp, Homo sapiens
aaatccctccatagtgatggaagaatgagccccagagagaagaatgtttctaatgaatcactggattgtgatataggattaacttggtgtccctaataccatttttttttcctcctgaaagtttaaggtcttatgtttaggaactagtttctctccaccttaatcctttattgtcaagtctgcaataatgttaagaacaggaaaaaaaaaatgtagattcctggataggcacagtttttatattaatgtaactatataggcatagtttttatattaatgtaactatacagcacctatttttgtgttttactattacttggcagacatcttgagtgttttacaaggttatcgtatatttcactaataatcgttgcttgataatttggtgtcctgacagactgc

SEQ ID NO: 290 - DNA, 403 bp, Homo sapiens; identical to SEQ ID NO: 288

SEQ ID NO: 291 - DNA, 402 bp, Homo sapiens; identical to SEQ ID NO: 287

SEQ ID NO: 292 - DNA, 404 bp, Homo sapiens
atagaacaatgcctagcacatagtagagatacataatcactactactactgctaccagtacaacagcaggtcttatggacctaaggtcatataacttagtctcttccaagattcttgaaatgatttctcaaaacaagagaatataaagaagaaacgttatgaacaaatggtaaataagaataaatgttagtaataaatggtaaaaaaaaaaaaaaaaggatatgaaagccaatagttacatgttctttcctgttaaagctattttacaaatggaaggaagcaaatttactttttcctcttgaacccgtgaactttgaaaatcttctcatctatttgactgagtagtatggtcttttaaatggtatataagataagaagtattcaaaataaagatatagccttta

SEQ ID NO: 293 - DNA, 402 bp, Homo sapiens
atagaacaatgcctagcacatagtagagatacataatcactactactactgctaccagtacaacagcaggtcttatggacctaaggtcatataacttagtctcttccaagattcttgaaatgatttctcaaaacaagagaatataaagaagaaacgttatgaacaaatggtaaataagaataaatgttagtaataaatggtaaaaaaaaaaaaaaggatatgaaagccaatagttacatgttctttcctgttaaagctattttacaaatggaaggaagcaaatttactttttcctcttgaacccgtgaactttgaaaatcttctcatctatttgactgagtagtatggtcttttaaatggtatataagataagaagtattcaaaataaagatatagccttta

SEQ ID NO: 294 - DNA, 403 bp, Homo sapiens
atagaacaatgcctagcacatagtagagatacataatcactactactactgctaccagtacaacagcaggtcttatggacctaaggtcatataacttagtctcttccaagattcttgaaatgatttctcaaaacaagagaatataaagaagaaacgttatgaacaaatggtaaataagaataaatgttagtaataaatggtaaaaaaaaaaaaaaaggatatgaaagccaatagttacatgttctttcctgttaaagctattttacaaatggaaggaagcaaatttactttttcctcttgaacccgtgaactttgaaaatcttctcatctatttgactgagtagtatggtcttttaaatggtatataagataagaagtattcaaaataaagatatagccttta

SEQ ID NO: 295 - DNA, 405 bp, Homo sapiens
atagaacaatgcctagcacatagtagagatacataatcactactactactgctaccagtacaacagcaggtcttatggacctaaggtcatataacttagtctcttccaagattcttgaaatgatttctcaaaacaagagaatataaagaagaaacgttatgaacaaatggtaaataagaataaatgttagtaataaatggtaaaaaaaaaaaaaaaaaggatatgaaagccaatagttacatgttctttcctgttaaagctattttacaaatggaaggaagcaaatttactttttcctcttgaacccgtgaactttgaaaatcttctcatctatttgactgagtagtatggtcttttaaatggtatataagataagaagtattcaaaataaagatatagccttta

SEQ ID NO: 296 - DNA, 402 bp, Homo sapiens
tttagaatgtactgtataggtgatttgtgggggtaacaaacctaaataatttaaagtagtctttatttgctgagaactgcaggtttttttaaagtatattttaaatctttaaactttcagagattaagagagattggccagggatttatttggagcaggaatttctttttcttgtgcttgcgtctttcccagcatccattcttttttgtgcctccatctagaatcatgtaatgtcagcgctagaagagaccaaagacagccatcctttacagcagtagttttcagatttcttttacagccaaatcctttatgcaaaaaaaaaaaaaaaaaaaagtgccactagcaataaaacagggaaaaccagagttacagctgtcctggttggggcttctttgtcccctc

SEQ ID NO: 297 - DNA, 417 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacaacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 298 - DNA, 415 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacaacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 299 - DNA, 418 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 300 - DNA, 420 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacacacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 301 - DNA, 424 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 302 - DNA, 422 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 303 - DNA, 420 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 304 - DNA, 418 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 305 - DNA, 420 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacacactctcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 306 - DNA, 416 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 307 - DNA, 418 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacacactctcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 308 - DNA, 414 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 309 - DNA, 416 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacacactctcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 310 - DNA, 412 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 311 - DNA, 414 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacacactctcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 312 - DNA, 410 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 313 - DNA, 412 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacactctcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 314 - DNA, 418 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacacacacacgcaaacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 315 - DNA, 402 bp, Homo sapiens
ctttcttttatttaactgaaatctgcctcccattaacatctctcattggtcctgattcggcctcaggaggatcacagagctgatgcaattctctttccttatgcaggtatgtttgcaaccactgctgccaaaatggcctctgccatcttttacctagttggttttttttgaaaatgaacacacacacacacacacacacactcacacacacacactcacagaaatatcctcttattgactgcaatccatctttcaacatatggagttctttttgaatactagaggtatagccttaaagaatatgagtatcagaagatactttagtttcatctttccctgcctgattcatcagccaattgttagtatgccatcagtcaagccattaataaaaataatgaacaa

SEQ ID NO: 316 - DNA, 402 bp, Homo sapiens
cctggccaacatggtgaaaccccatctctactaaaaatacaaaaaatgagccaggcatggtggcaggcgcctgtgatcccagctactcaggaggttgagacaggagaatcacttgaacctgggaggtggaggttgcagtgagctgaggccgcaccactgcactccagcctgggcaacagagtgagactctgtctcaaagaaaaaaaaaaaaaaaagaaaagaaaaaaaagaaatattgtctggctaaagaaaggaaaagaattcttattcagaatcagcatgatcacttctgggaatctgaatggagaaaataattctatatgtaatgatgttttcaatcaatattattttaggtggtttatttattcagccaactgttgttggctgcccactgtataccag

SEQ ID NO: 317 - DNA, 403 bp, Homo sapiens
cctggccaacatggtgaaaccccatctctactaaaaatacaaaaaatgagccaggcatggtggcaggcgcctgtgatcccagctactcaggaggttgagacaggagaatcacttgaacctgggaggtggaggttgcagtgagctgaggccgcaccactgcactccagcctgggcaacagagtgagactctgtctcaaagaaaaaaaaaaaaaaaaagaaaagaaaaaaaagaaatattgtctggctaaagaaaggaaaagaattcttattcagaatcagcatgatcacttctgggaatctgaatggagaaaataattctatatgtaatgatgttttcaatcaatattattttaggtggtttatttattcagccaactgttgttggctgcccactgtataccag

SEQ ID NO: 318 - DNA, 402 bp, Homo sapiens
cctggccaacatggtgaaaccccatctctactaaaaatacaaaaaatgagccaggcatggtggcaggcgcctgtgatcccagctactcaggaggttgagacaggagaatcacttgaacctgggaggtggaggttgcagtgagctgaggccgcaccactgcactccagcctgggcaacagagtgagactctgtctcaaagaacaaaaaaaaaaaaagaaaagaaaaaaaagaaatattgtctggctaaagaaaggaaaagaattcttattcagaatcagcatgatcacttctgggaatctgaatggagaaaataattctatatgtaatgatgttttcaatcaatattattttaggtggtttatttattcagccaactgttgttggctgcccactgtataccag

SEQ ID NO: 319 - DNA, 404 bp, Homo sapiens
cctggccaacatggtgaaaccccatctctactaaaaatacaaaaaatgagccaggcatggtggcaggcgcctgtgatcccagctactcaggaggttgagacaggagaatcacttgaacctgggaggtggaggttgcagtgagctgaggccgcaccactgcactccagcctgggcaacagagtgagactctgtctcaaagaacaaaaaaaaaaaaaaagaaaagaaaaaaaagaaatattgtctggctaaagaaaggaaaagaattcttattcagaatcagcatgatcacttctgggaatctgaatggagaaaataattctatatgtaatgatgttttcaatcaatattattttaggtggtttatttattcagccaactgttgttggctgcccactgtataccag

SEQ ID NO: 320 - DNA, 407 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 321 - DNA, 403 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 322 - DNA, 405 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 323 - DNA, 409 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 324 - DNA, 411 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 325 - DNA, 413 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 326 - DNA, 415 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 327 - DNA, 417 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 328 - DNA, 419 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 329 - DNA, 421 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattgtgtgtgtgtgtgtgtgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 330 - DNA, 404 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctatttgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 331 - DNA, 411 bp, Homo sapiens
actttcccatttatctagtgatgctatatgcattatcacatttaatgcttaaaacttgagctattgttatccctattctaacaagataatcaaagcatggagaaattaactctgtcttgctaagatcctcagatatgttctgaatcataaaaggttatgttatatttagcacagtgtttatagtaagaatgttttctctattttgtgtgtgtgtgtgtgtgtgtgtgtgtgtgtgtgttggaataccattataatctataagtctctcttgattttcagctagtgtgactcccctttccataactcccacctcaaatttgaaccatcctgaatagagaggagctttaataaaccaactctattattggttctgtaagaccttgacatttgcaaactttattttttgccttt

SEQ ID NO: 332 - DNA, 401 bp, Homo sapiens
attttattacctatactcataagaattgtattataaaatacattgttaaacgaatgttttcagtgctccattgagagtcggtggagcacactggttgggagaagacagagctgtgagccatccgtctgcctgtgcttgagtcttggctctgccattgactagttgtatgaactgccgcaggtggttcagccactcagaacctctgtaaaagtgagatgtaaaaacactttctacatcataggattattgtgaagattaaatgtgatatgttgtaaaattctggtcacacaagtattaacttactgttatttttgctgccactgctattaattaatggcagtgtggcggctcagtactaggcaatgggcgtgcaactgtgatgagaaacgcttctgtccatt

SEQ ID NO: 333 - DNA, 407 bp, Homo sapiens; identical to SEQ ID NO: 189

SEQ ID NO: 334 - DNA, 409 bp, Homo sapiens
attttattacctatactcataagaattgtattataaaatacattgttaaacgaatgttttcagtgctccattgagagtcggtggagcacactggttgggagaagacagagctgtgagccatccgtctgcctgtgcttgagtcttggctctgccattgactagttgtatgaactgccgcaggtggttcagccactcagaacctcagtatctctgtaaaagtgagatgtaaaaacactttctacatcataggattattgtgaagattaaatgtgatatgttgtaaaattctggtcacacaagtattaacttactgttatttttgctgccactgctattaattaatggcagtgtggcggctcagtactaggcaatgggcgtgcaactgtgatgagaaacgcttctgtccatt

SEQ ID NO: 335 - DNA, 409 bp, Homo sapiens; identical to SEQ ID NO: 334

SEQ ID NO: 336 - DNA, 412 bp, Homo sapiens; identical to SEQ ID NO: 188

SEQ ID NO: 337 - DNA, 401 bp, Homo sapiens; identical to SEQ ID NO: 332

SEQ ID NO: 338 - DNA, 402 bp, Homo sapiens
tgagaaactggtggaccgacacactctaattttttggcttctgaccaaacaagctagaaggatgccaaaattcaacaaaataacacattattgtgtgataggagccgtgctccaagagagcaggaactcagaggaacttcatactggccccttttaaaaaagcattgtcactttggggagctttcttagagaaacgagaggaaaatggtaaaatgcaacctggagagtaaggtataatttgcacatgaacacgaaggaaggaactgaaagaaaacagaggagtttaaagttacttctatgaacttttcccagacataacacacagttctctgacttgacttacattcttttaaccctgaaagttccatctctgtgtctgagcagaatgctggactgcttaac

SEQ ID NO: 339 - DNA, 402 bp, Homo sapiens
tttctgcctgctcctttaattcctcttggaaagtttacggttaatattttccctggaacattgtcaagcttttgacagtgcctgagtgtatgccgaactgtgaaattgagccggagaagcaagttgtgagaaatctgtttctactcagatccgtaaggtttatgggggggggaaaaaaaaccaaaaaaaaaaaaaaaaacccaaaaaaacaaaacaaaacaaaaaacaaaaaacttcagaggggaaactgagaatgggactcggcttgcttctcctggtgtgggttcaggccgccattttaaggagccagtgaagggcgacgttccgctccttacatggcggctgtatttactcggccgcagccaatcagccggcagtgccaagccacgtgacatgccacga

SEQ ID NO: 340 - DNA, 402 bp, Homo sapiens
tttctgcctgctcctttaattcctcttggaaagtttacggttaatattttccctggaacattgtcaagcttttgacagtgcctgagtgtatgccgaactgtgaaattgagccggagaagcaagttgtgagaaatctgtttctactcagatccgtaaggtttatgggggggggaaaaaaaaccaaaaaaaaaaaaaaaaaccaaaaaaaacaaaacaaaacaaaaaacaaaaaacttcagaggggaaactgagaatgggactcggcttgcttctcctggtgtgggttcaggccgccattttaaggagccagtgaagggcgacgttccgctccttacatggcggctgtatttactcggccgcagccaatcagccggcagtgccaagccacgtgacatgccacga

SEQ ID NO: 341 - DNA, 401 bp, Homo sapiens
ggcatgcagtgaggagcacctttgtagctagaacatgcttagattttggtattcttgaaaatgtggcctcctccccaatgccagtgtataggatttaaaaaaaaacaaaaaaacacatctcaaaccttggcatttattgaatattaacaggccaggcaccaaagcattattcagcattgacacttaaacttttctgtattgattattattattattattattttttgagacaggatctcactctcttgcccaggctggagtacagtgacataatcttggctcactacaacttgtgcctcccaggctcaagtgattctcttgcctcagtcttttgagtagctgggactacaagctcgcaccaccacacccatctaatttttgtattttttgtagagacgggc

SEQ ID NO: 342 - DNA, 404 bp, Homo sapiens
ggcatgcagtgaggagcacctttgtagctagaacatgcttagattttggtattcttgaaaatgtggcctcctccccaatgccagtgtataggatttaaaaaaaaacaaaaaaacacatctcaaaccttggcatttattgaatattaacaggccaggcaccaaagcattattcagcattgacacttaaacttttctgtattgattattattattattattattattttttgagacaggatctcactctcttgcccaggctggagtacagtgacataatcttggctcactacaacttgtgcctcccaggctcaagtgattctcttgcctcagtcttttgagtagctgggactacaagctcgcaccaccacacccatctaatttttgtattttttgtagagacgggc

SEQ ID NO: 343 - DNA, 421 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgtgtgtgtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 344 - DNA, 401 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 345 - DNA, 403 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 346 - DNA, 405 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 347 - DNA, 407 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 348 - DNA, 411 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 349 - DNA, 413 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 350 - DNA, 421 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgagtgtgtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 351 - DNA, 421 bp, Homo sapiens
tgcactttgagtgtgggaaacagtatgtgggtacataaacaaaataatttctaatgtgatagatctctaaaggaaacaggcaaggtgatagagaataactaagaggacctgctttagatgggaatgtgaaggatgaggccgcattcatactaagcatccaagtaaggagaagaccaagtgcaaaaagtttggtcgggatgagtgtgtgtgtgtgtgtgtgagtgtgtgtgtgtgtgtgtgtgtgtgtgtgtaccagagactgaaggccattatggctggacagttaagggagagtgacctaaataaagttcatattggcgggagagcagcttgccacagggttacacagaataggcagtggtggtaaaggcagcatttgaatccaggtccatttggctttggaatgtgctgttatagcagc

SEQ ID NO: 352 - DNA, 402 bp, Homo sapiens
tagagttctcaagagatctggtagttcaaaagtgtgtggcaccttcccctcccctctctctctccctctctgccatgtgaagaaggtgctcacttacactttgccttctgccatgagtgtaagtttcctgaggcctccccagccatgcttcctgtacagcctttggaactgtgagtcaattaaaccttttcttcataaattaaaaaaaaagaaagaaagaaaatttaatgacagtctaggctccccattagtgagacatgtcctcagtgaagtaagtgcaacttgtaacaacaataattcatcttcctagactccataaaggaaagaacattgcttttagcttggttttgaccttcacctttagggaccaccactaccatcagcccctgccatcattatgcc

SEQ ID NO: 353 - DNA, 405 bp, Homo sapiens
tgattttactgggttatttatttatttttagagacagggtcttgctctacaacccaggccggatttcagtgatgcatccatagctcattgtaacctcaaactcctgagtttaagtgatcctcctgcctcagaacctaagcacctgggactacaggcatgtgccaccataccaggctaatatatatatatatatatatttttttatttttttatttttttattttttgtagagactgtgtctttcctacgttgctcaggctgctcttgaactctaccctccgaaagtactgggattacaggcatgatccacaggacccagccctagatcttctatttttgattgtgaaataacctctgattgtgaagacacctgctttaagagcttttttcccaaaagaattgtga

SEQ ID NO: 354 - DNA, 405 bp, Homo sapiens
tgattttactgggttatttatttatttttagagacagggtcttgctctacaacccaggccggatttcagtgatgcatccatagctcattgtaacctcaaactcctgagtttaagtgatcctcctgcctcagaacctaagcacctgggactacaggcatgtgccaccataccaggctaatatatatatatatatatatttttatatttttttatttttttattttttgtagagactgtgtctttcctacgttgctcaggctgctcttgaactctaccctccgaaagtactgggattacaggcatgatccacaggacccagccctagatcttctatttttgattgtgaaataacctctgattgtgaagacacctgctttaagagcttttttcccaaaagaattgtga

SEQ ID NO: 355 - DNA, 405 bp, Homo sapiens
tgattttactgggttatttatttatttttagagacagggtcttgctctacaacccaggccggatttcagtgatgcatccatagctcattgtaacctcaaactcctgagtttaagtgatcctcctgcctcagaacctaagcacctgggactacaggcatgtgccaccataccaggctaatatatatatatatatatattttttaatttttttatttttttattttttgtagagactgtgtctttcctacgttgctcaggctgctcttgaactctaccctccgaaagtactgggattacaggcatgatccacaggacccagccctagatcttctatttttgattgtgaaataacctctgattgtgaagacacctgctttaagagcttttttcccaaaagaattgtga

SEQ ID NO: 356 - DNA, 406 bp, Homo sapiens
tgattttactgggttatttatttatttttagagacagggtcttgctctacaacccaggccggatttcagtgatgcatccatagctcattgtaacctcaaactcctgagtttaagtgatcctcctgcctcagaacctaagcacctgggactacaggcatgtgccaccataccaggctaatatatatatatatatatatttttttattttttttatttttttattttttgtagagactgtgtctttcctacgttgctcaggctgctcttgaactctaccctccgaaagtactgggattacaggcatgatccacaggacccagccctagatcttctatttttgattgtgaaataacctctgattgtgaagacacctgctttaagagcttttttcccaaaagaattgtga

SEQ ID NO: 357 - DNA, 405 bp, Homo sapiens
tgattttactgggttatttatttatttttagagacagggtcttgctctacaacccaggccggatttcagtgatgcatccatagctcattgtaacctcaaactcctgagtttaagtgatcctcctgcctcagaacctaagcacctgggactacaggcatgtgccaccataccaggctaatatatatatatatatatatttttttttttttttatttttttattttttgtagagactgtgtctttcctacgttgctcaggctgctcttgaactctaccctccgaaagtactgggattacaggcatgatccacaggacccagccctagatcttctatttttgattgtgaaataacctctgattgtgaagacacctgctttaagagcttttttcccaaaagaattgtga

SEQ ID NO: 358 - DNA, 405 bp, Homo sapiens
caaaacaacaacaaatatacatatacacttacatttccctaaagaaatattgagataatatacaaaaactaataaaaggatttaccaaaagggagatgggaaatggagtggacagagatgcagctatgagctatgagcaagttttctcaatgagtatatttatatcattttcatttttgaacagtattgtctattcaaaataaacaaaattctgccacagattagggggaaaataagaatagtctctttgatggggatggccatgtgcatatctctcagaaatcccacatggggagcaggaggctaggacttccaggtggcatagcattttcaacacaagtcacgttcatcacaaggtgggggaatcatcagagggttcctttgatggatgggatgtggaggtgg

SEQ ID NO: 359 - DNA, 414 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaattgtgtgtgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 360 - DNA, 401 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaatgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 361 - DNA, 407 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaatgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 362 - DNA, 409 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaatgtgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 363 - DNA, 411 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaatgtgtgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 364 - DNA, 413 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaatgtgtgtgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 365 - DNA, 415 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaatgtgtgtgtgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 366 - DNA, 410 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaattgtgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 367 - DNA, 412 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaattgtgtgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

SEQ ID NO: 368 - DNA, 416 bp, Homo sapiens
ctttttattgtttcctttaaatagcaattagggaagatagcactccattttgcctcctacttgcccttttgctaaatcatgatttcaccctgtgccagatagttatgggtgtatgaaaagatggcactggtgaaaggcagagcggtgaacacacttgactcaagcctgaggaatccaggaaaaagttgccaatgatgaaaattgtgtgtgtgtgtgtgtgtgtgtgtgtgtgtgcgcgtccacatgtgtgtgtagtgaataccttagaacaattcctttattcacatattcagaagtgtaaaacatgcctatttggaagtacagattcacttacataatgtctaccagtgtgctgcagttatttaaaagctagctatcaacttggtaagatatgggaacttttctattttgtacct

(19) United States
(12) Patent Application Publication
(10) Pub. No.: US 2014/0087961 A1
(43) Pub. Date: Mar. 27, 2014

(54) GENETIC VARIANTS USEFUL FOR RISK ASSESSMENT OF THYROID CANCER

(71) Applicants: Patrick Sulem, Reykjavik (IS); Julius Gudmundsson, Reykjavik (IS)

(72) Inventors: Patrick Sulem, Reykjavik (IS); Julius Gudmundsson, Reykjavik (IS)

(73) Assignees: ILLUMINA INC., San Diego, CA (US); deCODE Genetics ehf., Reykjavik (IS)

(21) Appl. No.: 14/004,359

(22) PCT Filed: Mar. 16, 2012
(86) PCT No.: PCT/IS12/50006

§ 371 Date: Sep. 10, 2013

(30) Foreign Application Priority Data
Mar. 17, 2011 (IS) 050012
Jan. 20, 2012 (IS) 050027

(51) Int. Cl. C12Q 1/68

(52) U.S. Cl. CPC C12Q 1/6886
USPC 506/9; 435/6.12; 435/6.11; 435/287.2; 506/16

(57) ABSTRACT

The invention discloses genetic variants that have been determined to be susceptibility variants of thyroid cancer. Methods of disease management, including methods of determining susceptibility to thyroid cancer, methods of predicting response to therapy and methods of predicting prognosis of thyroid cancer using such variants are described. The invention further relates to kits useful in the methods of the invention.

### INTRODUCTION

Thyroid carcinoma is the most common classical endocrine malignancy, and its incidence has been rising rapidly in the US as well as other industrialized countries over the past few decades. Thyroid cancers are classified histologically into four groups: papillary, follicular, medullary, and undifferentiated or anaplastic thyroid carcinomas (DeLellis, R. A., J Surg Oncol, 94, 662 (2006)). In 2008, it is expected that over 37,000 new cases will be diagnosed in the US, about 75% of them being females (the ratio of males to females is 1:3.2) (Jemal, A., et al., Cancer statistics, 2008. CA Cancer J Clin, 58: 71-96, (2008)). If diagnosed at an early stage, thyroid cancer is a well manageable disease with a 5-year survival rate of 97% among all patients, yet about 1,600 individuals were expected to die from this disease in 2008 in the US (Jemal, A., et al., Cancer statistics, 2008. CA Cancer J Clin, 58: 71-96, (2008)). Survival rate is poorer (˜40%) among individuals that are diagnosed with a more advanced disease; i.e. individuals with large, invasive tumors and/or distant metastases have a 5-year survival rate of ≈40% (Sherman, S. I., et al., 3rd, Cancer, 83, 1012 (1998), Kondo, T., Ezzat, S., and Asa, S. L., Nat Rev Cancer, 6, 292 (2006)). For radioiodine-resistant metastatic disease there is no effective treatment and the 10-year survival rate among these patients is less than 15% (Durante, C., et al., J Clin Endocrinol Metab, 91, 2892 (2006)).

Although relatively rare (1% of all malignancies in the US), the incidence of thyroid cancer more than doubled between 1984 and 2004 in the US (SEER web report; Ries L, Melbert D, Krapcho M et al (2007) SEER cancer statistics review, 1975-2004. National Cancer Institute, Bethesda, Md., http://seer.cancer.gov/csr/1975_{—}2004/, based on November 2006 SEER data submission). Between 1995 and 2004, thyroid cancer was the third fastest growing cancer diagnosis, behind only peritoneum, omentum, and mesentery cancers and “other” digestive cancers [SEER web report]. Similarly dramatic increases in thyroid cancer incidence have also been observed in Canada, Australia, Israel, and several European countries (Liu, S., et al., Br J Cancer, 85, 1335 (2001), Burgess, J. R., Thyroid, 12, 141 (2002), Lubina, A., et al., Thyroid, 16, 1033 (2006), Colonna, M., et al., Eur J Cancer, 38, 1762 (2002), Leenhardt, L., et al., Thyroid, 14, 1056 (2004), Reynolds, R. M., et al., Clin Endocrinol (Oxf), 62, 156 (2005), Smailyte, G., et al., BMC Cancer, 6, 284 (2006)).

Thus, there is a need for better understanding of the molecular causes of thyroid cancer progression, to develop new diagnostic tools and better treatment options. The present invention provides thyroid cancer susceptibility variants and their use in various diagnostic applications.

### SUMMARY OF THE INVENTION

The present invention relates to methods of risk management of thyroid cancer, based on the discovery that certain genetic variants are correlated with risk of thyroid cancer. Thus, the invention includes methods of determining an increased susceptibility or increased risk of thyroid cancer, as well as methods of determining a decreased susceptibility of thyroid cancer, through evaluation of certain markers that have been found to be correlated with susceptibility of thyroid cancer in humans. Other aspects of the invention relate to methods of assessing prognosis of individuals diagnosed with thyroid cancer, methods of assessing the probability of response to a therapeutic agents or therapy for thyroid cancer, as well as methods of monitoring progress of treatment of individuals diagnosed with thyroid cancer.

In one aspect, the invention relates to a method of determining a susceptibility to Thyroid Cancer, the method comprising analyzing nucleic acid sequence data from a human individual for at least one polymorphic marker selected from the group consisting of rs334725, rs116909374, and rs28933981, and markers in linkage disequilibrium therewith, wherein different alleles of the at least one polymorphic marker are associated with different susceptibilities to Thyroid Cancer in humans, and determining a susceptibility to Thyroid Cancer from the nucleic acid sequence data.

In another aspect, the invention relates to a method of determining a susceptibility to thyroid cancer in a human individual, the method comprising determining the presence or absence of at least one allele of at least one polymorphic marker selected from the group consisting of the markers rs334725, rs116909374, and rs28933981, and markers in linkage disequilibrium therewith, in a nucleic acid sample obtained from the individual, wherein the presence of the at least one allele is indicative of a susceptibility to thyroid cancer.

The invention also relates to a method of determining a susceptibility to thyroid cancer, the method comprising determining the presence or absence of at least one allele of at least one polymorphic marker selected from the group consisting of the markers rs334725, rs116909374, and rs28933981, and markers in linkage disequilibrium therewith, wherein the determination of the presence of the at least one allele is indicative of a susceptibility to thyroid cancer.

In another aspect the invention further relates to a method for determining a susceptibility to thyroid cancer in a human individual, comprising determining whether at least one allele of at least one polymorphic marker is present in a nucleic acid sample obtained from the individual, or in a genotype dataset derived from the individual, wherein the at least one polymorphic marker is selected from the group consisting of markers rs334725, rs116909374, and rs28933981, and markers in linkage disequilibrium therewith, and wherein the presence of the at least one allele is indicative of a susceptibility to thyroid cancer for the individual.

The invention further relates to a method of determining a susceptibility to Thyroid Cancer, the method comprising analyzing nucleic acid sequence data from a human individual for at least one polymorphic marker selected within the human transthyretin (TTR) gene, wherein different alleles of the at least one polymorphic marker are associated with different susceptibilities to Thyroid Cancer in humans, and determining a susceptibility to Thyroid Cancer from the nucleic acid sequence data. In one embodiment, the at least one polymorphic marker is selected from the group consisting of rs28933981, and markers in linkage disequilibrium therewith.

The invention also provides a method of identification of a marker for use in assessing susceptibility to Thyroid Cancer in human individuals, the method comprising (i) identifying at least one polymorphic marker in linkage disequilibrium with at least one of rs334725, rs116909374, and rs28933981; (ii) obtaining sequence information about the at least one polymorphic marker in a group of individuals diagnosed with Thyroid Cancer; and (iii) obtaining sequence information about the at least one polymorphic marker in a group of control individuals; wherein determination of a significant difference in frequency of at least one allele in the at least one polymorphism in individuals diagnosed with Thyroid Cancer as compared with the frequency of the at least one allele in the control group is indicative of the at least one polymorphism being useful for assessing susceptibility to Thyroid Cancer.

Further provided are prognostic methods and methods of assessing probability to treatment. Thus, a further aspect of the invention relates to a method of predicting prognosis of an individual diagnosed with Thyroid Cancer, the method comprising obtaining sequence data about a human individual about at least one polymorphic marker selected from the group consisting of rs334725, rs116909374, and rs28933981, and markers in linkage disequilibrium therewith, wherein different alleles of the at least one polymorphic marker are associated with different susceptibilities to Thyroid Cancer in humans, and predicting prognosis of the Thyroid Cancer from the sequence data. Also provided is a method of assessing probability of response of a human individual to a therapeutic agent for preventing, treating and/or ameliorating symptoms associated with Thyroid Cancer, comprising obtaining sequence data about a human individual identifying at least one allele of at least one polymorphic marker selected from the group consisting of rs334725, rs116909374, and rs28933981, and markers in linkage disequilibrium therewith, wherein different alleles of the at least one polymorphic marker are associated with different probabilities of response to the therapeutic agent in humans, and determining the probability of a positive response to the therapeutic agent from the sequence data.

The invention also provides kits. In one such aspect, the invention relates to a kit for assessing susceptibility to Thyroid Cancer in human individuals, the kit comprising reagents for selectively detecting at least one at-risk variant for Thyroid Cancer in the individual, wherein the at least one at-risk variant is selected from the group consisting of rs334725, rs116909374, and rs28933981, and markers in linkage disequilibrium therewith, and a collection of data comprising correlation data between the at least one at-risk variant and susceptibility to Thyroid Cancer.

Further provided is the use of an oligonucleotide probe in the manufacture of a diagnostic reagent for diagnosing and/or assessing a susceptibility to Thyroid Cancer, wherein the probe is capable of hybridizing to a nucleic acid segment with sequence as set forth in any one of SEQ ID NO:1-210, and wherein the nucleic acid segment is 15-400 nucleotides in length.

The invention also provides computer-implemented applications. In one such application, the invention relates to an apparatus for determining a susceptibility to Thyroid Cancer in a human individual, comprising a processor and a computer readable memory having computer executable instructions adapted to be executed on the processor to analyze information for at least one human individual with respect to at least one marker selected from the group consisting of rs334725, rs116909374, and rs28933981, and markers in linkage disequilibrium therewith, and generate an output based on the marker or amino acid information, wherein the output comprises at least one measure of susceptibility to Thyroid Cancer for the human individual.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

FIG. 2 provides a diagram illustrating a system comprising computer implemented methods utilizing risk variants as described herein.

FIG. 3 shows an exemplary system for determining risk of thyroid cancer as described further herein.

FIG. 4 shows a system for selecting a treatment protocol for a subject diagnosed with thyroid cancer.

FIG. 5 shows the unadjusted (diamonds) and adjusted (circle) thyroid cancer association results (−log 10 P-value) for rs944289 (left) and rs116909374 (right), as well as the recombination rate in 375 kb region on 14q13.3. The recombination rate (cM/Mb) is based on CEU HapMap phase II release 22. The association results are the combined unadjusted and adjusted results for the four study groups reported in Table 5.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein, including sequence listing, is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

The symbol or “-” as disclosed in Tables 7 and 8 herein, refers to multiple alleles as specified in the accompanying sequencing listing for the particular marker, excluding the opposite allele. For example marker rs77363846 (Seq ID no 108) in Table 7 has risk allele C and the other allele can be either CT or CCT, designated as “-” in Table 7.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles. Haplotypes are described herein in the context of the marker name and the allele of the marker in that haplotype, e.g., “2 rs334725” refers to the 2 allele of marker rs334725 being in the haplotype, and is equivalent to “rs334725 allele 2”. Furthermore, allelic codes in haplotypes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of thyroid cancer, as characterized by a relative risk (RR) or odds ratio (OR) of greater than one for the particular allele or haplotype. Alternatively, the markers and/or haplotypes of the invention are characteristic of decreased susceptibility (i.e., decreased risk) of thyroid cancer, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “thyroid cancer therapeutic agent” refers to an agent that can be used to ameliorate or prevent symptoms associated with thyroid cancer.

The term “thyroid cancer-associated nucleic acid”, as described herein, refers to a nucleic acid that has been found to be associated to thyroid cancer. This includes, but is not limited to, the markers and haplotypes described herein and markers and haplotypes in strong linkage disequilibrium (LD) therewith. In one embodiment, a thyroid cancer-associated nucleic acid refers to a genomic region, such as an LD-block, found to be associated with risk of thyroid cancer through at least one polymorphic marker located within the region or LD block.

### Variants Associated with Risk of Thyroid Cancer

The present inventors have identified genomic regions that contain markers that correlate with risk of thyroid cancer. On chromosome 14q13.3, a region exemplified by marker rs116909374 (SEQ ID NO:43) has been found to correlate with risk of thyroid cancer. Further, a region on chromosome 1p31.3, exemplified by marker rs334725 (SEQ ID NO:3), and a region on chromosome 18q12.1, exemplified by marker rs28933981 (SEQ ID NO:53) in the transthyretin gene (TTR) has been found to associate with risk of thyroid cancer. Markers in these regions are useful for assessing genetic risk of thyroid cancer in human individuals. The rs28933981 marker encodes a missense variation in human TTR. Thus, the at-risk T allele of rs28933981 encodes a Threonine to Methionine substitution (T139M) at position 139 in an encoded TTR protein (Genbank Accession Number: CAG33189).

As a consequence, the present invention in one aspect provides a method of determining a susceptibility to Thyroid Cancer, the method comprising analyzing nucleic acid sequence data from a human individual for at least one polymorphic marker selected from the group consisting of rs116909374, rs334725 and rs28933981, and markers in linkage disequilibrium therewith, wherein different alleles of the at least one polymorphic marker are associated with different susceptibilities to Thyroid Cancer in humans, and determining a susceptibility to Thyroid Cancer from the nucleic acid sequence data.

In certain embodiments, suitable surrogate markers are markers that are correlated to at least one of rs334725, rs116909374 and/or rs28933981 by values of r² of at least 0.2. Markers are selected from the group consisting of markers in linkage disequilibrium with rs334725 characterized by values of the linkage disequilibrium measure r² of greater than 0.2. In another preferred embodiment, suitable markers are selected from the group consisting of markers in linkage disequilibrium with rs116909374 characterized by values of the linkage disequilibrium measure r² of greater than 0.2. In certain other preferred embodiment, suitable polymorphic markers are selected from markers that are correlated with rs334725, rs28933981 and/or rs116909374 by values of the linkage disequilibrium measure r² of greater than 0.8.

Certain alleles of risk variants of thyroid cancer are predictive of increased risk (increased susceptibility) of thyroid cancer. Thus, the C allele of rs334725, the T allele of rs116909374 and the T allele of rs28933981 are alleles indicative of increased risk of thyroid cancer (at-risk alleles). Thus, in certain embodiment, determination of the presence of at least one allele selected from the group consisting of the C allele of rs334725, the T allele of rs116909374 and the T allele of rs28933981 is indicative of increased risk of thyroid cancer for the individual. Other risk alleles of thyroid cancer that are correlated with the T allele of rs116909374 are listed in Table 8 herein. The risk alleles listed in the Table are also predictive of thyroid cancer. Thus, certain embodiments of the invention pertain to the particular risk alleles listed in Table 8 herein. Likewise, risk alleles of thyroid cancer that are correlated with the C allele of rs334725, which is equal to the G allele of rs334725 on the reverse strand of DNA, are listed in Table 7 herein. These alleles are therefore also predictive of risk of thyroid cancer. Accordingly, certain embodiments of the invention pertain to the use of the risk alleles listed in Table 7 herein.

Determination of the absence of any one of these risk alleles is indicative that the individual does not have the increased risk conferred by the allele. In certain other embodiments, alleles indicative of risk of thyroid cancer are selected from the group consisting of the marker alleles listed in Table 1 that are correlated with the at-risk C allele of rs334725. In certain embodiments, such risk allels are selected from the risk alleles listed in Table 7 herein. In certain other embodiments, alleles indicative of risk of thyroid cancer are selected from the group consisting of the marker alleles listed in Table 2 that are correlated with the at-risk T allele of rs116909374. In certain such embodiments, the alleles indicative or risk of thyroid cancer are selected from the risk alleles listed in Table 8 herein.

As will be described in more detail in the below, the skilled person will appreciate that marker alleles in linkage disequilibrium with any one of these at-risk alleles of thyroid cancer are also predictive of increased risk of thyroid cancer, and may thus also be suitably selected for use in the methods of the invention.

The allele that is detected can suitably be the allele of the complementary strand of DNA, such that the nucleic acid sequence data includes the identification of at least one allele which is complementary to any of the alleles of the polymorphic markers referenced above. For example, the allele that is detected may be the complementary G allele of the at-risk C allele of rs334725. The allele that is detected may also be the complementary A allele of the at-risk T allele of rs116909374. The allele that is detected may also be the complementary A allele of the at-risk T allele of rs28933981.

In certain embodiments, the nucleic acid sequence data is obtained from a biological sample containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; (iii) hybridization assay using a nucleic acid probe and nucleic acid obtained by amplification of the biological sample, and (iv) nucleic acid sequencing, in particular high-throughput sequencing. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to thyroid cancer.

In another aspect, a method is provided that comprises (1) obtaining a sample containing nucleic acid from a human individual; (2) obtaining nucleic acid sequence data about at least one polymorphic marker in the sample, wherein different alleles of the at least one marker are associated with different susceptibilities of thyroid cancer in humans; (3) analyzing the nucleic acid sequence data about the at least one marker; and (4) determining a risk of thyroid cancer from the nucleic acid sequence data. In certain embodiments, the analyzing comprises determining the presence or absence of at least one allele of the at least one polymorphic marker.

It is contemplated that in certain embodiments of the invention, it may be convenient to prepare a report of results of risk assessment. Thus, certain embodiments of the methods of the invention comprise a further step of preparing a report containing results from the determination, wherein said report is written in a computer readable medium, printed on paper, or displayed on a visual display. In certain embodiments, it may be convenient to report results of susceptibility to at least one entity selected from the group consisting of the individual, a guardian of the individual, a genetic service provider, a physician, a medical organization, and a medical insurer.

In another aspect, the invention relates to a method of determining a susceptibility to thyroid cancer in a human individual, comprising determining whether at least one at-risk allele in at least one polymorphic marker is present in a genotype dataset derived from the individual, wherein the at least one polymorphic marker is selected from the group consisting of the markers rs334725, rs116909374 and rs28933981, and markers in linkage disequilibrium therewith, and wherein determination of the presence of the at least one at-risk allele is indicative of increased susceptibility to thyroid cancer in the individual.

A genotype dataset derived from an individual is in the present context a collection of genotype data that is indicative of the genetic status of the individual for particular genetic markers. The dataset is derived from the individual in the sense that the dataset has been generated using genetic material from the individual, or by other methods available for determining genotypes at particular genetic markers (e.g., imputation methods). The genotype dataset comprises in one embodiment information about marker identity and the allelic status of the individual for at least one allele of a marker, i.e. information about the identity of at least one allele of the marker in the individual. The genotype dataset may comprise allelic information (information about allelic status) about one or more marker, including two or more markers, three or more markers, five or more markers, ten or more markers, one hundred or more markers, and so on. In some embodiments, the genotype dataset comprises genotype information from a whole-genome assessment of the individual, which may include hundreds of thousands of markers, or even one million or more markers spanning the entire genome of the individual.

Another aspect of the invention relates to a method of determining a susceptibility to thyroid cancer in a human individual, the method comprising obtaining nucleic acid sequence data about a human individual identifying at least one allele of at least one polymorphic marker selected from the group consisting of the markers rs334725, rs116909374 and rs28933981, and markers in linkage disequilibrium therewith, wherein different alleles of the at least one polymorphic marker are associated with different susceptibilities to thyroid cancer in humans, and determining a susceptibility to thyroid cancer from the nucleic acid sequence data.

In certain embodiments, the sequence data is analyzed using a computer processor to determine a susceptibility to thyroid cancer from the sequence data. Alternatively, the sequence data is transformed into a risk measure of thyroid cancer for the individual.

Obtaining nucleic acid sequence data may comprise steps of obtaining a biological sample from the human individual and transforming the sample to analyze sequence of the at least one polymorphic marker in the sample. Alternatively, sequence data obtained from a dataset may be transformed. Any suitable method known to the skilled artisan for obtaining a biological sample may be used, for example using the methods described herein. Likewise, transforming the sample to analyze sequence may be performed using any method known to the skilled artisan, including the methods described herein for determining disease risk.

### Assessment of Other Biomarkers for Thyroid Cancer

Certain embodiments of the invention further comprise assessing the quantitative levels of a biomarker for thyroid cancer. For example, the levels of a biomarker may be determined in concert with analysis of particular genetic markers. Alternatively, biomarker levels are determined at a different point in time, but results of such determination are used together with results from sequencing analysis for particular polymorphic markers. The biomarker may in some embodiments be assessed in a biological sample from the individual. In some embodiments, the sample is a blood sample. The blood sample is in some embodiments a serum sample. In preferred embodiments, the biomarker is selected from the group consisting of thyroid stimulating hormone (TSH), thyroxine (T4) and thriiodothyronine (T3). In certain embodiments, determination of an abnormal level of the biomarker is indicative of an abnormal thyroid function in the individual, which may in turn be indicative of an increased risk of thyroid cancer in the individual. The abnormal level can be an increased level or the abnormal level can be a decreased level. In certain embodiments, the determination of an abnormal level is determined based on determination of a deviation from the average levels of the biomarker in the population. In one embodiment, abnormal levels of TSH are measurements of less than 0.2 mIU/L and/or greater than 10 mIU/L. In another embodiment, abnormal levels of TSH are measurements of less than 0.3 mIU/L and/or greater than 3.0 mIU/L. In another embodiment, abnormal levels of T₃ (free T₃) are less than 70 ng/dL and/or greater than 205 ng/dL. In another embodiment, abnormal levels of T₄ (free T₄) are less than 0.8 ng/dL and/or greater than 2.7 ng/dL.

The markers conferring risk of thyroid cancer, as described herein, can be combined with other genetic markers for thyroid cancer. Such markers are typically not in linkage disequilibrium with rs334725, rs116909374 and rs28933981, or other markers in linkage disequilibrium with those markers. Any of the methods described herein can be practiced by combining the genetic risk factors described herein with additional genetic risk factors for thyroid cancer.

Thus, in certain embodiments, a further step is included, comprising determining whether at least one at-risk allele of at least one at-risk variant for thyroid cancer not in linkage disequilibrium with any one of the markers rs334725, rs116909374 and rs28933981, or markers in linkage disequilibrium therewith, is present in a sample comprising genomic DNA from a human individual or a genotype dataset derived from a human individual. In other words, genetic markers in other locations in the genome can be useful in combination with the markers of the present invention, so as to determine overall risk of thyroid cancer based on multiple genetic variants. Selection of markers that are not in linkage disequilibrium (not in LD) can be based on a suitable measure for linkage disequilibrium, as described further herein. In certain embodiments, markers that are not in linkage disequilibrium have values of the LD measure r² correlating the markers of less than 0.2. In certain other embodiments, markers that are not in LD have values for r² correlating the markers of less than 0.15, including less than 0.10, less than 0.05, less than 0.02 and less than 0.01. Other suitable numerical values for establishing that markers are not in LD are contemplated, including values bridging any of the above-mentioned values.

In one embodiment, assessment of one or more of the markers described herein is combined with assessment of at least one marker selected from the group consisting of marker rs965513 on chromosome 9q22, marker rs944289 on chromosome 14q13, marker rs7005606 on chromosome 8p12 and marker rs966423 on chromosome 2q35, or a marker in linkage disequilibrium therewith, to establish overall risk. In certain such embodiments, determination of the presence of the A allele of rs965513, the T allele of rs944289, the G allele of rs7005606 and/or the C allele of rs966423 is indicative of increased risk of thyroid cancer. In one embodiment, the A allele of rs965513 is an at-risk allele of thyroid cancer, the T allele of rs944289 is an at-risk allele of thyroid cancer, the G allele of rs7005606 is an at-risk allele of thyroid cancer and the C allele of rs966423 is an at-risk allele of thyroid cancer.

In certain embodiments, multiple markers as described herein are determined to determine overall risk of thyroid cancer. Thus, in certain embodiments, an additional step is included, the step comprising determining whether at least one allele in each of at least two polymorphic markers is present in a sample comprising genomic DNA from a human individual or a genotype dataset derived from a human individual, wherein the presence of the at least one allele in the at least two polymorphic markers is indicative of an increased susceptibility to thyroid cancer.

The genetic markers of the invention can also be combined with non-genetic information to establish overall risk for an individual. Thus, in certain embodiments, a further step is included, comprising analyzing non-genetic information to make risk assessment, diagnosis, or prognosis of the individual. The non-genetic information can be any information pertaining to the disease status of the individual or other information that can influence the estimate of overall risk of thyroid cancer for the individual. In one embodiment, the non-genetic information is selected from age, gender, ethnicity, socioeconomic status, previous disease diagnosis, medical history of subject, family history of thyroid cancer, biochemical measurements, and clinical measurements.

### Obtaining Nucleic Acid Sequence Data

Sequence data can be nucleic acid sequence data, which may be obtained by means known in the art. Sequence data is suitably obtained from a biological sample of genomic DNA, RNA, or cDNA (a “test sample”) from an individual (“test subject). For example, nucleic acid sequence data may be obtained through direct analysis of the sequence of the polymorphic position (allele) of a polymorphic marker. Suitable methods, some of which are described herein, include, for instance, whole genome sequencing methods, whole genome analysis using SNP chips (e.g., Infinium HD BeadChip), cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis; heteroduplex analysis, chemical mismatch cleavage (CMC), RNase protection assays, use of polypeptides that recognize nucleotide mismatches, such as E. coli mutS protein, allele-specific PCR, and direct manual and automated sequencing. These and other methods are described in the art (see, for instance, Li et al., Nucleic Acids Research, 28(2): e1 (i-v) (2000); Liu et al., Biochem Cell Bio 80:17-22 (2000); and Burczak et al., Polymorphism Detection and Analysis, Eaton Publishing, 2000; Sheffield et al., Proc. Natl. Acad. Sci. USA, 86:232-236 (1989); Orita et al., Proc. Natl. Acad. Sci. USA, 86:2766-2770 (1989); Flavell et al., Cell, 15:25-41 (1978); Geever et al., Proc. Natl. Acad. Sci. USA, 78:5081-5085 (1981); Cotton et al., Proc. Natl. Acad. Sci. USA, 85:4397-4401 (1985); Myers et al., Science 230:1242-1246 (1985); Church and Gilbert, Proc. Natl. Acad. Sci. USA, 81:1991-1995 (1984); Sanger et al., Proc. Natl. Acad. Sci. USA, 74:5463-5467 (1977); and Beavis et al., U.S. Pat. No. 5,288,644).

Recent technological advances have resulted in technologies that allow massive parallel sequencing to be performed in relatively condensed format. These technologies share sequencing-by-synthesis principle for generating sequence information, with different technological solutions implemented for extending, tagging and detecting sequences. Exemplary technologies include 454 pyrosequencing technology (Nyren, P. et al. Anal Biochem 208:171-75 (1993); http://www.454.com), Illumina Solexa sequencing technology (Bentley, D. R. Curr Opin Genet Dev 16:545-52 (2006); http://www.illumina.com), and the SOLID technology developed by Applied Biosystems (ABI) (http://www.appliedbiosystems.com; see also Strausberg, R. L., et al. Drug Disc Today 13:569-77 (2008)). Other sequencing technologies include those developed by Pacific Biosciences (http://www.pacificbiosciences.com), Complete Genomics (http://www.completegenomics.com), Intelligen Bio-Systems (http://www.intelligentbiosystems.com), Genome Corp (http://www.genomecorp.com), ION Torrent Systems (http://www.iontorrent.com) and Helicos Biosciences (http://www.helicosbio.com). It is contemplated that sequence data useful for performing the present invention may be obtained by any such sequencing method, or other sequencing methods that are developed or made available. Thus, any sequence method that provides the allelic identity at particular polymorphic sites (e.g., the absence or presence of particular alleles at particular polymorphic sites) is useful in the methods described and claimed herein.

Alternatively, hybridization methods may be used (see Current Protocols in Molecular Biology, Ausubel et al., eds., John Wiley & Sons, including all supplements). For example, a biological sample of genomic DNA, RNA, or cDNA (a “test sample”) may be obtained from a test subject. The subject can be an adult, child, or fetus. The DNA, RNA, or cDNA sample is then examined. The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to Thyroid Cancer, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 10, 15, 30, 50, 100, 250 or 500 nucleotides in length that is sufficient to specifically hybridize under stringent conditions to appropriate mRNA or genomic DNA. In certain embodiments, the nucleic acid probe is capable of hybridizing to a nucleic acid with sequence as set forth in any one of SEQ ID NO:1-210. Hybridization can be performed by methods well known to the person skilled in the art (see, e.g., Current Protocols in Molecular Biology, Ausubel et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen et al., Bioconjug. Chem. 5:3-7 (1994)). The PNA probe can be designed to specifically hybridize to a molecule in a sample suspected of containing one or more of the marker alleles that are associated with risk of thyroid cancer.

In one embodiment of the invention, a test sample containing genomic DNA obtained from the subject is collected and the polymerase chain reaction (PCR) is used to amplify a fragment comprising one or more polymorphic marker. As described herein, identification of particular marker alleles can be accomplished using a variety of methods. In another embodiment, determination of a susceptibility is accomplished by expression analysis, for example using quantitative PCR (kinetic thermal cycling). This technique can, for example, utilize commercially available technologies, such as TaqMan® (Applied Biosystems, Foster City, Calif.). The technique can for example assess the presence of an alteration in the expression or composition of a polypeptide or splicing variant(s) that is encoded by a nucleic acid associated described herein. Alternatively, this technique may assess expression levels of genes or particular splice variants of genes, that are affected by one or more of the variants described herein. Further, the expression of the variant(s) can be quantified as physically or functionally different.

Allele-specific oligonucleotides can also be used to detect the presence of a particular allele in a nucleic acid. An “allele-specific oligonucleotide” (also referred to herein as an “allele-specific oligonucleotide probe”) is an oligonucleotide of any suitable size, for example an oligonucleotide of approximately 10-50 base pairs or approximately 15-30 base pairs, that specifically hybridizes to a nucleic acid which contains a specific allele at a polymorphic site (e.g., a polymorphic marker). An allele-specific oligonucleotide probe that is specific for one or more particular alleles at polymorphic markers can be prepared using standard methods (see, e.g., Current Protocols in Molecular Biology, supra). PCR can be used to amplify the desired region. Specific hybridization of an allele-specific oligonucleotide probe to DNA from a subject is indicative of the presence of a specific allele at a polymorphic site (see, e.g., Gibbs et al., Nucleic Acids Res. 17:2437-2448 (1989) and WO 93/22456).

With the addition of analogs such as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tm) of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′ end, or in the middle), the Tm could be increased considerably. It is therefore contemplated that in certain embodiments, LNAs are used to detect particular alleles at polymorphic sites associated with thyroid cancer, as described herein.

In certain embodiments, arrays of oligonucleotide probes that are complementary to target nucleic acid sequence segments from a subject, can be used to identify polymorphisms in a nucleic acid. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier et al., Adv Biochem Eng Biotechnol 109:433-53 (2008); Hoheisel, Nat Rev Genet 7:200-10 (2006); Fan et al., Methods Enzymol 410:57-73 (2006); Raqoussis & Elvidge, Expert Rev Mol Diagn 6:145-52 (2006); Mockler et al., Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Also, standard techniques for genotyping can be used to detect particular marker alleles, such as fluorescence-based techniques (e.g., Chen et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave).

Suitable biological sample in the methods described herein can be any sample containing nucleic acid (e.g., genomic DNA) and/or protein from the human individual. For example, the biological sample can be a blood sample, a serum sample, a leukapheresis sample, an amniotic fluid sample, a cerbrospinal fluid sample, a hair sample, a tissue sample from skin, muscle, buccal, or conjuctival mucosa, placenta, gastrointestinal tract, or other organs, a semen sample, a urine sample, a saliva sample, a nail sample, a tooth sample, and the like. Preferably, the sample is a blood sample, a salive sample or a buccal swab.

### Protein Analysis

Missense nucleic acid variations may lead to an altered amino acid sequence, as compared to the non-variant (e.g., wild-type) protein, due to one or more amino acid substitutions, deletions, or insertions, or truncation (due to, e.g., splice variation). In such instances, detection of the amino acid substitution of the variant protein may be useful. This way, nucleic acid sequence data may be obtained through indirect analysis of the nucleic acid sequence of the allele of the polymorphic marker, i.e. by detecting a protein variation. Methods of detecting variant proteins are known in the art. For example, direct amino acid sequencing of the variant protein followed by comparison to a reference amino acid sequence can be used. Alternatively, SDS-PAGE followed by gel staining can be used to detect variant proteins of different molecular weights. Also, Immunoassays, e.g., immunofluorescent immunoassays, immunoprecipitations, radioimmunoassays, ELISA, and Western blotting, in which an antibody specific for an epitope comprising the variant sequence among the variant protein and non-variant or wild-type protein can be used. In certain embodiments of the present invention, the T139M substitution in TTR is detected in a protein sample. The detection may be suitably performed using any of the methods described in the above.

In some cases, a variant protein has altered (e.g., upregulated or downregulated) biological activity, in comparison to the non-variant or wild-type protein. The biological activity can be, for example, a binding activity or enzymatic activity. In this instance, altered biological activity may be used to detect a variation in protein encoded by a nucleic acid sequence variation. Methods of detecting binding activity and enzymatic activity are known in the art and include, for instance, ELISA, competitive binding assays, quantitative binding assays using instruments such as, for example, a Biacore® 3000 instrument, chromatographic assays, e.g., HPLC and TLC.

Alternatively or additionally, a protein variation encoded by a genetic variation could lead to an altered expression level, e.g., an increased expression level of an mRNA or protein, a decreased expression level of an mRNA or protein. In such instances, nucleic acid sequence data about the allele of the polymorphic marker, or protein sequence data about the protein variation, can be obtained through detection of the altered expression level. Methods of detecting expression levels are known in the art. For example, ELISA, radioimmunoassays, immunofluorescence, and Western blotting can be used to compare the expression of protein levels. Alternatively, Northern blotting can be used to compare the levels of mRNA. These processes are described in Sambrook et al., Molecular Cloning: A Laboratory Manual, 3^{rd} ed. Cold Spring Harbor Laboratory Press, Cold Spring Harbor, N.Y. (2001).

Any of these methods may be performed using a nucleic acid (e.g., DNA, mRNA) or protein of a biological sample obtained from the human individual for whom a susceptibility is being determined. The biological sample can be any nucleic acid or protein containing sample obtained from the human individual. For example, the biological sample can be any of the biological samples described herein.

It is further contemplated that additional missense variants in human TTR protein may be association with thyroid cancer risk. The present invention thus also encompasses methods of determining susceptibility of thyroid cancer, using further missense variants in human TTR that confer risk of thyroid cancer.

### Number of Polymorphic Markers/Genes Analyzed

With regard to the methods of determining a susceptibility described herein, the methods can comprise obtaining sequence data about any number of polymorphic markers and/or about any number of genes. For example, the method can comprise obtaining sequence data for about at least 1, 2, 3, 4, 5, 6, 7, 8, 9, 10, 15, 20, 25, 30, 40, 50, 100, 500, 1000, 10,000 or more polymorphic markers. In certain embodiments, the sequence data is obtained from a microarray comprising probes for detecting a plurality of markers. The markers can be independent of rs334725, rs116909374 and rs28933981 and/or the markers may be in linkage disequilibrium with rs334725, rs116909374 and rs28933981. The polymorphic markers can be the ones of the group specified herein or they can be different polymorphic markers that are not listed herein. In a specific embodiment, the method comprises obtaining sequence data about at least two polymorphic markers. In certain embodiments, each of the markers may be associated with a different gene. For example, in some instances, if the method comprises obtaining nucleic acid data about a human individual identifying at least one allele of a polymorphic marker, then the method comprises identifying at least one allele of at least one polymorphic marker. Also, for example, the method can comprise obtaining sequence data about a human individual identifying alleles of multiple, independent markers, which are not in linkage disequilibrium.

### Linkage Disequilibrium

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrance of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The correlation measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots.

For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one specific embodiment of invention, the significant r² value can be at least 0.2. In another specific embodiment of invention, the significant r² value can be at least 0.5. In one specific embodiment of invention, the significant r² value can be at least 0.8. Alternatively, linkage disequilibrium as described herein, refers to linkage disequilibrium characterized by values of r² of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). Linkage disequilibrium can be determined in a single human population, as defined herein, or it can be determined in a collection of samples comprising individuals from more than one human population. In one embodiment of the invention, LD is determined in a sample from one or more of the HapMap populations. These include samples from the Yoruba people of Ibadan, Nigeria (YRI), samples from individuals from the Tokyo area in Japan (JPT), samples from individuals Beijing, China (CHB), and samples from U.S. residents with northern and western European ancestry (CEU), as described (The International HapMap Consortium, Nature 426:789-796 (2003)). In one such embodiment, LD is determined in the Caucasian CEU population of the HapMap samples. In another embodiment, LD is determined in the African YRI population. In yet another embodiment, LD is determined in samples from the Icelandic population.

If all polymorphisms in the genome were independent at the population level (i.e., no LD between polymorphisms), then every single one of them would need to be investigated in association studies, to assess all different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the invention.

By way of example, the markers rs334725, rs116909374 and/or rs28933981 may be detected directly to determine risk of Thyroid Cancer. Alternatively, any marker in linkage disequilibrium with rs334725, rs116909374 and/or rs28933981, in particular markers that are closely correlated with rs334725, rs116909374 and/or rs28933981, may be detected to determine risk.

The present invention thus refers to the markers rs334725, rs116909374 and/or rs28933981 for detecting association to Thyroid Cancer, as well as markers in linkage disequilibrium with these markers. Thus, in certain embodiments of the invention, markers that are in LD with these markers, e.g., markers as described herein, may be used as surrogate markers.

Suitable surrogate markers may be selected using public information, such as from the International HapMap Consortium (http://www.hapmap.org) and the International 1000genomes Consortium (http://www.1000genomes.org). Publically available software may be used to identify suitable surrogate markers, for example markers that fulfill selected criteria of the LD measures r² and D′. One such software tool is available through the Broad Institute (http://www.broadinstitute.org/mpg/snap/Idsearch.php). The stronger the linkage disequilibrium, in particular in terms of the correlation coefficient r², to the anchor marker, the better the surrogate, and thus the mores similar the association detected by the surrogate is expected to be to the association detected by the anchor marker. Markers with values of r² equal to 1 are perfect surrogates for the at-risk variants, i.e. genotypes for one marker perfectly predicts genotypes for the other. In other words, the surrogate will, by necessity, give exactly the same association data to any particular disease as the anchor marker. Markers with smaller values of r² than 1 can also be surrogates for the at-risk anchor variant.

The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and select appropriate surrogate markers.

In certain embodiments, suitable surrogate markers of rs334725 are selected from the group consisting of the markers set forth in Table 1 and Table 7. In certain embodiments, suitable surrogate markers of rs116909374 are selected from the group consisting of the markers set forth in Table 2 and Table 8. In one preferred embodiment, surrogate markers of rs334725 are selected from the group consisting of the markers set forth in Table 7. In one preferred embodiment, surrogate markers of rs116909374 are selected from the group consisting of the markers set forth in Table 8.

In general, and as further described herein, surrogate markers will be selected from the appropriate population, i.e. the population in which it is of interest to practice the invention described herein for particular diagnostic purpose. For example, if the invention is to be practiced in white individuals, it is suitable to select surrogate markers, when applicable, from a population of white individuals. In certain embodiments, suitable surrogate markers are selected in European Americans, i.e. Americans of European origin. In certain embodiments, suitable surrogate marker are selected in samples from European populations. In certain embodiments, suitable surrogate marker are selected in samples from Caucasians. In certain embodiments, it may be suitable to select surrogate markers from the Icelandic population. Other embodiments relate to surrogate markers selected in any particular human population, e.g. Chinese, Japanese, Russian, and so on, as described further herein.

TABLE 1
Surrogate markers for anchor marker rs334725 on Chromosome
1p31.3. Shown are marker names, position in NCBI Build 36, r²
values, and SEQ ID for flanking sequence of the marker.

| Name | Position in NCBI | r² | SEQ ID NO: |
|---|---|---|---|
| rs10493302 | 61343980 | 0.248 | 1 |
| rs3748543 | 61368577 | 0.952 | 2 |
| rs334725 | 61382637 | 1 | 3 |
| rs334709 | 61385776 | 0.827 | 4 |
| rs334708 | 61386184 | 0.493 | 5 |
| rs334707 | 61388124 | 0.547 | 6 |
| rs334706 | 61388835 | 0.97 | 7 |
| s334704 | 61389682 | 0.956 | 8 |
| rs334703 | 61390107 | 1 | 9 |
| rs334702 | 61391281 | 0.819 | 10 |
| rs334701 | 61391644 | 0.704 | 11 |
| rs334700 | 61392051 | 0.914 | 12 |
| rs334699 | 61393084 | 1 | 13 |
| rs334698 | 61393581 | 0.929 | 14 |
| rs334713 | 61394875 | 0.873 | 15 |
| rs334712 | 61395343 | 0.748 | 16 |
| rs334711 | 61397898 | 0.481 | 17 |
| rs334710 | 61398460 | 0.906 | 18 |
| rs75117939 | 61399126 | 0.571 | 19 |
| rs334715 | 61400019 | 0.553 | 20 |
| rs168022 | 61402041 | 0.619 | 21 |
| rs914735 | 61419013 | 0.252 | 22 |
| rs80195615 | 61419091 | 0.249 | 23 |
| rs12091215 | 61419691 | 0.267 | 24 |
| rs12086591 | 61419744 | 0.283 | 25 |
| rs12081195 | 61419756 | 0.266 | 26 |
| rs55916522 | 61421101 | 0.246 | 27 |
| rs55718193 | 61421104 | 0.236 | 28 |
| rs79484896 | 61423301 | 0.244 | 29 |
| rs12065271 | 61423409 | 0.259 | 30 |
| rs79529781 | 61424069 | 0.229 | 31 |
| rs17121791 | 61424221 | 0.231 | 32 |
| rs17121793 | 61424334 | 0.267 | 33 |
| rs17121794 | 61424408 | 0.279 | 34 |
| rs1332780 | 61426024 | 0.232 | 35 |
| rs11207708 | 61426709 | 0.226 | 36 |
| rs115882681 | 61440442 | 0.335 | 37 |

TABLE 2
Surrogates for anchor marker rs116909374 on Chromosome 14q13.3.
Shown are marker names or ID's (chromosome followed by location
in NCBI Build 36), position in NCBI Build 36, r² and SEQ
ID for flanking sequence of the marker.

| | Position in NCBI | | |
|---|---|---|---|
| Name or Chr: Pos | Bld 36 | r² | SEQ ID NO: |
| chr14: 35686997 | 35686997 | 0.209 | 38 |
| rs61994967 | 35771779 | 0.219 | 39 |
| rs116955509 | 35782720 | 0.276 | 40 |
| rs17104226 | 35799615 | 0.233 | 41 |
| rs78485296 | 35802958 | 0.238 | 42 |
| rs116909374 | 35808112 | 1 | 43 |
| rs17175276 | 35847635 | 0.269 | 44 |
| chr14: 35850167 | 35850167 | 0.37 | 45 |
| chr14: 35902878 | 35902878 | 0.265 | 46 |
| chr14: 35916596 | 35916596 | 0.264 | 47 |
| chr14: 35957607 | 35957607 | 0.244 | 48 |
| chr14: 35971477 | 35971477 | 0.247 | 49 |
| chr14: 35992635 | 35992635 | 0.25 | 50 |
| chr14: 36147091 | 36147091 | 0.214 | 51 |
| chr14: 36202933 | 36202933 | 0.235 | 52 |

### Association analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, for example a cohort from a different population (e.g., different region of same country, or a different country) of the same or different ethnicity. The advantage of replication studies is that the number of tests performed in the replication study is usually quite small, and hence the less stringent the statistical measure that needs to be applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold (i.e., more significant) are a measure of a true genetic effect, and replication in additional cohorts is not necessary from a statistical point of view. Importantly, however, signals with P-values that are greater than this threshold may also be due to a true genetic effect. The sample size in the first study may not have been sufficiently large to provide an observed P-value that meets the conservative threshold for genome-wide significance, or the first study may not have reached genome-wide significance due to inherent fluctuations due to sampling. Since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

### Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study.

Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### Determining Risk

In the present context, an individual who is at an increased susceptibility (i.e., increased risk) for Thyroid Cancer is an individual who is carrying at least one at-risk allele in marker rs334725, marker rs116909374 or marker rs28933981. Alternatively, an individual who is at an increased susceptibility for Thyroid Cancer is an individual who is carrying at least one at-risk allele in a marker that is correlated with rs334725, rs116909374 or rs28933981. In one embodiment, significance associated with a marker is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 1.10, including but not limited to: at least 1.15, at least 1.20, at least 1.25, at least 1.30, at least 1.35, at least 1.40, at least 1.45, at least 1.50, at least 1.55, at least 1.60, and at least 1.65. In a particular embodiment, a risk (relative risk and/or odds ratio) of at least 1.25 is significant. In another particular embodiment, a risk of at least 1.30 is significant.

An at-risk polymorphic marker as described herein is one where at least one allele of at least one marker is more frequently present in an individual diagnosed with, or at risk for, Thyroid Cancer (affected), compared to the frequency of its presence in a comparison group (control), such that the presence of the marker allele is indicative of increased susceptibility to Thyroid Cancer. The control group may in one embodiment be a population sample, i.e. a random sample from the general population. In another embodiment, the control group is represented by a group of individuals who are disease-free, i.e. individuals who have not been diagnosed with Thyroid Cancer.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

### Database

Determining susceptibility can alternatively or additionally comprise comparing nucleic acid sequence data and/or genotype data to a database containing correlation data between polymorphic markers and susceptibility to Thyroid Cancer. The database can be part of a computer-readable medium described herein.

In a specific aspect of the invention, the database comprises at least one measure of susceptibility to thyroid cancer for the polymorphic markers. For example, the database may comprise risk values associated with particular genotypes at such markers. The database may also comprise risk values associated with particular genotype combinations for multiple such markers.

In another specific aspect of the invention, the database comprises a look-up table containing at least one measure of susceptibility to thyroid cancer for the polymorphic markers.

### Further Steps

The methods disclosed herein can comprise additional steps which may occur before, after, or simultaneously with one of the aforementioned steps of the method of the invention. In a specific embodiment of the invention, the method of determining a susceptibility to Thyroid Cancer further comprises reporting the susceptibility to at least one entity selected from the group consisting of the individual, a guardian of the individual, a genetic service provider, a physician, a medical organization, and a medical insurer. The reporting may be accomplished by any of several means. For example, the reporting can comprise sending a written report on physical media or electronically or providing an oral report to at least one entity of the group, which written or oral report comprises the susceptibility. Alternatively, the reporting can comprise providing the at least one entity of the group with a login and password, which provides access to a report comprising the susceptibility posted on a password-protected computer system.

### Study Population

In a general sense, the methods and kits described herein can be utilized from samples containing nucleic acid material (DNA or RNA) from any source and from any individual, or from genotype or sequence data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers in individuals who are members of a target population. Such a target population is in one embodiment a population or group of individuals at risk of developing Thyroid Cancer, based on other genetic factors, biomarkers, biophysical parameters, history of Thyroid Cancer, family history of Thyroid Cancer or a related disease. In certain embodiments, a target population is a population with abnormal levels (high or low) of TSH, T4 or T3.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Sulem, P., et al. Nat Genet May 17, 2009 (Epub ahead of print); Rafnar, T., et al. Nat Genet 41:221-7 (2009); Gretarsdottir, S., et al. Ann Neurol 64:402-9 (2008); Stacey, S. N., et al. Nat Genet 40:1313-18 (2008); Gudbjartsson, D. F., et al. Nat Genet 40:886-91 (2008); Styrkarsdottir, U., et al. N Engl J Med 358:2355-65 (2008); Thorgeirsson, T., et al. Nature 452:638-42 (2008); Gudmundsson, 3., et al. Nat Genet. 40:281-3 (2008); Stacey, S. N., et al., Nat Genet. 39:865-69 (2007); Helgadottir, A., et al., Science 316:1491-93 (2007); Steinthorsdottir, V., et al., Nat Genet. 39:770-75 (2007); Gudmundsson, 3., et al., Nat Genet. 39:631-37 (2007); Frayling, T M, Nature Reviews Genet 8:657-662 (2007); Amundadottir, L. T., et al., Nat Genet. 38:652-58 (2006); Grant, S. F., et al., Nat Genet. 38:320-23 (2006)). Thus, genetic findings in the Icelandic population have in general been replicated in other populations, including populations from Africa and Asia.

It is thus believed that the markers described herein to be associated with risk of Thyroid Cancer will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, and Asian populations.

The racial contribution in individual subjects may also be determined by genetic analysis. Genetic analysis of ancestry may be carried out using unlinked microsatellite markers such as those set out in Smith et al. (Am J Hum Genet 74, 1001-13 (2004)).

In certain embodiments, the invention relates to markers identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as taught herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Screening Methods

The invention also provides a method of screening candidate markers for assessing susceptibility to Thyroid Cancer. The invention also provides a method of identification of a marker for use in assessing susceptibility to Thyroid Cancer. The method may comprise analyzing the frequency of at least one allele of a polymorphic marker in a population of human individuals diagnosed with Thyroid Cancer, wherein a significant difference in frequency of the at least one allele in the population of human individuals diagnosed with Thyroid Cancer as compared to the frequency of the at least one allele in a control population of human individuals is indicative of the allele as a marker of the Thyroid Cancer. In certain embodiments, the candidate marker is a marker in linkage disequilibrium with marker rs334725, marker rs116909374 or marker rs28933981.

In one embodiment, the method comprises (i) identifying at least one polymorphic marker in linkage disequilibrium, as determined by values of r² of greater than 0.5, with marker rs334725, marker rs116909374 or marker rs28933981; (ii) obtaining sequence information about the at least one polymorphic marker in a group of individuals diagnosed with Thyroid Cancer; and (iii) obtaining sequence information about the at least one polymorphic marker in a group of control individuals; wherein determination of a significant difference in frequency of at least one allele in the at least one polymorphism in individuals diagnosed with Thyroid Cancer as compared with the frequency of the at least one allele in the control group is indicative of the at least one polymorphism being useful for assessing susceptibility to Thyroid Cancer.

In one embodiment, an increase in frequency of the at least one allele in the at least one polymorphism in individuals diagnosed with Thyroid Cancer, as compared with the frequency of the at least one allele in the control group, is indicative of the at least one polymorphism being useful for assessing increased susceptibility to Thyroid Cancer. In another embodiment, a decrease in frequency of the at least one allele in the at least one polymorphism in individuals diagnosed with Thyroid Cancer, as compared with the frequency of the at least one allele in the control group, is indicative of the at least one polymorphism being useful for assessing decreased susceptibility to, or protection against, Thyroid Cancer.

### Thyroid Stimulating Hormone

Thyroid-stimulating hormone (also known as TSH or thyrotropin) is a peptide hormone synthesized and secreted by thyrotrope cells in the anterior pituitary gland which regulates the endocrine function of the thyroid gland. TSH stimulates the thyroid gland to secrete the hormones thyroxine (T₄) and triiodothyronine (T₃). TSH production is controlled by a Thyrotropin Releasing Hormone, (TRH), which is manufactured in the hypothalamus and transported to the anterior pituitary gland via the superior hypophyseal artery, where it increases TSH production and release. Somatostatin is also produced by the hypothalamus, and has an opposite effect on the pituitary production of TSH, decreasing or inhibiting its release.

The level of thyroid hormones (T₃ and T₄) in the blood have an effect on the pituitary release of TSH; when the levels of T₃ and T₄ are low, the production of TSH is increased, and conversely, when levels of T₃ and T₄ are high, then TSH production is decreased. This effect creates a regulatory negative feedback loop.

Thyroxine, or 3,5,3′,5′-tetraiodothyronine (often abbreviated as T₄), is the major hormone secreted by the follicular cells of the thyroid gland. T₄ is transported in blood, with 99.95% of the secreted T₄ being protein bound, principally to thyroxine-binding globulin (TBG), and, to a lesser extent, to transthyretin and serum albumin. T₄ is involved in controlling the rate of metabolic processes in the body and influencing physical development. Administration of thyroxine has been shown to significantly increase the concentration of nerve growth factor in the brains of adult mice.

In the hypothalamus, T₄ is converted to Triiodothyronine, also known as T₃. TSH is inhibited mainly by T₃. The thyroid gland releases greater amounts of T₄ than T₃, so plasma concentrations of T₄ are 40-fold higher than those of T₃. Most of the circulating T₃ is formed peripherally by deiodination of T₄ (85%), a process that involves the removal of iodine from carbon 5 on the outer ring of T₄. Thus, T₄ acts as prohormone for T₃.

### Utility of Genetic Testing

As discussed in the above, the primary known risk factor for thyroid cancer is radiation exposure. Thyroid cancer incidence within the US has been rising for several decades (Davies, L. and Welch, H. G., Jama, 295, 2164 (2006)), which may be attributable to increased detection of sub-clinical cancers, as opposed to an increase in the true occurrence of thyroid cancer (Davies, L. and Welch, H. G., Jama, 295, 2164 (2006)). The introduction of ultrasonography and fine-needle aspiration biopsy in the 1980s improved the detection of small nodules and made cytological assessment of a nodule more routine (Rojeski, M. T. and Gharib, H., N Engl J Med, 313, 428 (1985), Ross, D. S., J Clin Endocrinol Metab, 91, 4253 (2006)). This increased diagnostic scrutiny may allow early detection of potentially lethal thyroid cancers. However, several studies report thyroid cancers as a common autopsy finding (up to 35%) in persons without a diagnosis of thyroid cancer (Bondeson, L. and Ljungberg, O., Cancer, 47, 319 (1981), Harach, H. R., et al., Cancer, 56, 531 (1985), Solares, C. A., et al., Am J Otolaryngol, 26, 87 (2005) and Sobrinho-Simoes, M. A., Sambade, M. C., and Goncalves, V., Cancer, 43, 1702 (1979)). This suggests that many people live with sub-clinical forms of thyroid cancer which are of little or no threat to their health.

Physicians use several tests to confirm the suspicion of thyroid cancer, to identify the size and location of the lump and to determine whether the lump is non-cancerous (benign) or cancerous (malignant). Blood tests such as the thyroid stimulating hormone (TSH) test check thyroid function.

TSH levels are tested in the blood of patients suspected of suffering from excess (hyperthyroidism), or deficiency (hypothyroidism) of thyroid hormone. Generally, a normal range for TSH for adults is between 0.2 and 10 uIU/mL (equivalent to mIU/L). The optimal TSH level for patients on treatment ranges between 0.3 to 3.0 mIU/L. The interpretation of TSH measurements depends also on what the blood levels of thyroid hormones (T₃ and T₄) are. The National Health Service in the UK considers a “normal” range to be more like 0.1 to 5.0 uIU/mL.

TSH levels for children normally start out much higher. In 2002, the National Academy of Clinical Biochemistry (NACB) in the United States recommended age-related reference limits starting from about 1.3-19 uIU/mL for normal term infants at birth, dropping to 0.6-10 uIU/mL at 10 weeks old, 0.4-7.0 uIU/mL at 14 months and gradually dropping during childhood and puberty to adult levels, 0.4-4.0 uIU/mL. The NACB also stated that it expected the normal (95%) range for adults to be reduced to 0.4-2.5 uIU/mL, because research had shown that adults with an initially measured TSH level of over 2.0 uIU/mL had an increased odds ratio of developing hypothyroidism over the [following] 20 years, especially if thyroid antibodies were elevated.

In general, both TSH and T₃ and T₄ should be measured to ascertain where a specific thyroid dysfunction is caused by primary pituitary or by a primary thyroid disease. If both are up (or down) then the problem is probably in the pituitary. If the one component (TSH) is up, and the other (T₃ and T₄) is down, then the disease is probably in the thyroid itself. The same holds for a low TSH, high T3 and T4 finding.

The knowledge of underlying genetic risk factors for thyroid cancer can be utilized in the application of screening programs for thyroid cancer. Thus, carriers of at-risk variants for thyroid cancer may benefit from more frequent screening than do non-carriers. Homozygous carriers of at-risk variants are particularly at risk for developing thyroid cancer.

It may be beneficial to determine TSH, T3 and/or T4 levels in the context of a particular genetic profile, e.g. the presence of particular at-risk alleles for thyroid cancer as described herein (e.g., rs334725 allele C and/or rs116909374 allele T). Since TSH, T3 and T4 are measures of thyroid function, a diagnostic and preventive screening program will benefit from analysis that includes such clinical measurements. For example, an abnormal (increased or decreased) level of TSH together with determination of the presence of an at-risk genetic variant for thyroid cancer (e.g., rs334725, rs28933981 and/or rs116909374) is indicative that an individual is at risk of developing thyroid cancer. In one embodiment, determination of a decreased level of TSH in an individual in the context of the presence of rs334725 allele C and/or rs116909374 allele T is indicative of an increased risk of thyroid cancer for the individual. In another embodiment, determination of an increased level of free T4 in an individual in the context of the presence of rs28933981 allele T is indicative of an increased risk of thyroid cancer for the individual.

Also, carriers may benefit from more extensive screening, including ultrasonography and/or fine needle biopsy. The goal of screening programs is to detect cancer at an early stage. Knowledge of genetic status of individuals with respect to known risk variants can aid in the selection of applicable screening programs. In certain embodiments, it may be useful to use the at-risk variants for thyroid cancer described herein together with one or more diagnostic tool selected from Radioactive Iodine (RAI) Scan, Ultrasound examination, CT scan (CAT scan), Magnetic Resonance Imaging (MRI), Positron Emission Tomography (PET) scan, Fine needle aspiration biopsy and surgical biopsy.

The invention provides in one diagnostic aspect a method for identifying a subject who is a candidate for further diagnostic evaluation for thyroid cancer, comprising the steps of (a) determining, in the genome of a human subject, the allelic identity of at least one polymorphic marker, wherein different alleles of the at least one marker are associated with different susceptibilities to thyroid cancer, and wherein the at least one marker is selected from the group consisting of rs334725, rs28933981 and rs116909374, and markers in linkage disequilibrium therewith; and (b) identifying the subject as a subject who is a candidate for further diagnostic evaluation for thyroid cancer based on the allelic identity at the at least one polymorphic marker. Thus, the identification of individuals who are at increased risk of developing thyroid cancer may be used to select those individuals for follow-up clinical evaluation, as described in the above.

### Prognostic Methods

In addition to the utilities described above, the polymorphic markers of the invention are useful in determining prognosis of a human individual experiencing symptoms associated with, or an individual diagnosed with, thyroid cancer. Accordingly, the invention provides a method of predicting prognosis of an individual experiencing symptoms associated with, or an individual diagnosed with, thyroid cancer. The method comprises analyzing sequence data about a human individual for at least one polymorphic marker selected from the group consisting of rs334725, rs28933981 and/or rs116909374, and markers in linkage disequilibrium therewith, wherein different alleles of the at least one polymorphic marker are associated with different susceptibilities thyroid cancer in humans, and predicting prognosis of the individual from the sequence data.

The prognosis can be any type of prognosis relating to the progression of thyroid cancer, and/or relating to the chance of recovering from thyroid cancer. The prognosis can, for instance, relate to the severity of the cancer, when the cancer may take place (e.g., the likelihood of recurrence), or how the cancer will respond to therapeutic treatment.

With regard to the prognostic methods described herein, the sequence data obtained to establish a prognostic prediction is suitably nucleic acid sequence data. For example, in one embodiment, determination of the presence of an at-risk allele of thyroid cancer (e.g., rs334725 allele C and/or rs116909374 allele T) is useful for prognostic applications. Suitable methods of detecting particular at-risk alleles are known in the art, some of which are described herein.

### Therapeutic Agents

Treatment options for thyroid cancer include current standard treatment methods and those that are in clinical trials.

Current treatment options for thyroid cancer include:

Surgery—including lobectomy, where the lobe in which thyroid cancer is found is removed, thyroidectomy, where all but a very small part of the thyroid is removed, total thyroidectomoy, where the entire thyroid is removed, and lymphadenectomoy, where lymph nodes in the neck that contain cancerous growth are removed;

Radiation therapy—including externation radiation therapy and internal radiation therapy using a radioactive compound. Radiation therapy may be given after surgery to remove any surviving cancer cells. Also, follicular and papillary thyroid cancers are sometimes treated with radioactive iodine (RAI) therapy;

Chemotherapy—including the use of oral or intravenous administration of the chemotherapy compound;

Thyroid hormone therapy—this therapy includes administration of drugs preventing generation of thyroid-stimulating hormone (TSH) in the body.

A number of clinical trials for thyroid cancer therapy and treatment are currently ongoing, including but not limited to trials for ¹⁸F-fluorodeoxyglucose (FluGlucoScan); ¹¹¹In-Pentetreotide (NeuroendoMedix); Combretastatin and Paclitaxel/Carboplatin in the treatment of anaplastic thyroid cancer, ¹³¹I with or without thyroid-stimulating hormone for post-surgical treatment, XL184-301 (Exelixis), Vandetanib (Zactima; Astra Zeneca), CS-7017 (Sankyo), Decitabine (Dacogen; 5-aza-2′-deoxycytidine), Irinotecan (Pfizer, Yakult Honsha), Bortezomib (Velcade; Millenium Pharmaceuticals); 17-AAG (17-N-Allylamino-17-demethoxygeldanamycin), Sorafenib (Nexavar, Bayer), recombinant Thyrotropin, Lenalidomide (Revlimid, Celgene), Sunitinib (Sutent), Sorafenib (Nexavar, Bayer), Axitinib (AG-013736, Pfizer), Valproic Acid (2-propylpentanoic acid), Vandetanib (Zactima, Astra Zeneca), AZD6244 (Astra Zeneca), Bevacizumab (Avastin, Genetech/Roche), MK-0646 (Merck), Pazopanib (GlaxoSmithKline), Aflibercept (Sanofi-Aventis & Regeneron Pharmaceuticals), and FR901228 (Romedepsin).

### Methods for Predicting Response to Therapeutic Agents

As is known in the art, individuals can have differential responses to a particular therapy (e.g., a therapeutic agent or therapeutic method). Pharmacogenomics addresses the issue of how genetic variations (e.g., the variants (markers and/or haplotypes) of the invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the invention), or therapeutic failure of the drug. Therefore, the variants of the invention may determine the manner in which a therapeutic agent and/or method acts on the body, or the way in which the body metabolizes the therapeutic agent.

Accordingly, in one embodiment, the presence of a particular allele at a polymorphic site (e.g., rs334725 allele C, rs28933981 allele T and/or rs116909374 allele T) is indicative of a different response, e.g. a different response rate, to a particular treatment modality, for thyroid cancer. This means that a patient diagnosed with thyroid cancer and carrying such risk alleles would respond better to, or worse to, a specific therapeutic, drug and/or other therapy used to treat the cancer. Therefore, the presence or absence of the marker allele could aid in deciding what treatment should be used for the patient. If the patient is positive for the marker allele, then the physician recommends one particular therapy, while if the patient is negative for the at least one allele of a marker, then a different course of therapy may be recommended (which may include recommending that no immediate therapy, other than serial monitoring for progression of symptoms, be performed). Thus, the patient's carrier status could be used to help determine whether a particular treatment modality should be administered. In one embodiment, the presence of an at-risk allele for thyroid cancer, e.g. rs334725 allele C, rs28933981 allele T and/or rs116909374 allele T, is indicative of a positive response to a particular therapy for thyroid cancer. In certain embodiments, the therapy is selected from the group consisting of surgery, radiation therapy, chemotherapy and thyroid hormone therapy.

Another aspect of the invention relates to methods of selecting individuals suitable for a particular treatment modality, based on their likelihood of developing particular complications or side effects of the particular treatment. It is well known that many therapeutic agents can lead to certain unwanted complications or side effects. Likewise, certain therapeutic procedures or operations may have complications associated with them. Complications or side effects of these particular treatments or associated with specific therapeutic agents can, just as diseases do, have a genetic component. It is therefore contemplated that selection of the appropriate treatment or therapeutic agent can in part be performed by determining the genotype of an individual, and using the genotype status (e.g., the presence or absence of rs334725 allele C, rs28933981 allele T and/or rs116909374 allele T) of the individual to decide on a suitable therapeutic procedure or on a suitable therapeutic agent to treat thyroid cancer. It is therefore contemplated that the polymorphic markers of the invention can be used in this manner. Indiscriminate use of a such therapeutic agents or treatment modalities may lead to unnecessary and needless adverse complications.

In view of the foregoing, the invention provides a method of assessing an individual for probability of response to a therapeutic agent for preventing, treating, and/or ameliorating symptoms associated thyroid cancer. In one embodiment, the method comprises: analyzing nucleic acid sequence data from a human individual for at least one polymorphic marker selected from the group consisting of rs334725, rs28933981 and rs116909374, and markers in linkage disequilibrium therewith, wherein determination of the presence of the rs334725 allele C, rs28933981 allele T and/or rs116909374 allele T, or a marker allele in linkage disequilibrium therewith, indicative of a probability of a positive response to the therapeutic agent.

In a further aspect, the markers of the invention can be used to increase power and effectiveness of clinical trials. Thus, individuals who are carriers of particular at-risk variants for thyroid cancer (e.g., rs334725 allele C, rs28933981 and/or rs116909374 allele T) may be more likely to respond to a particular treatment modality. For some treatments, the genetic risk may correlate with less responsiveness to therapy. This application can improve the safety of clinical trials, but can also enhance the chance that a clinical trial will demonstrate statistically significant efficacy, which may be limited to a certain sub-group of the population. Thus, one possible outcome of such a trial is that carriers of the at-risk markers of the invention are statistically significantly likely to show positive response to the therapeutic agent, i.e. experience alleviation of symptoms associated with thyroid cancer, when taking the therapeutic agent or drug as prescribed. Another possible outcome is that genetic carriers show less favorable response to the therapeutic agent, or show differential side-effects to the therapeutic agent compared to the non-carrier. An aspect of the invention is directed to screening for such pharmacogenetic correlations.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies, means for amplification of nucleic acids, means for analyzing the nucleic acid sequence of nucleic acids, means for analyzing the amino acid sequence of a polynucleotides, etc. The kits can for example include necessary buffers, nucleic acid primers for amplifying nucleic acids (e.g., a nucleic acid segment comprising one or more of the polymorphic markers as described herein), and reagents for allele-specific detection of the fragments amplified using such primers and necessary enzymes (e.g., dna polymerase). Additionally, kits can provide reagents for assays to be used in combination with the methods of the present invention, e.g., reagents for use with other diagnostic assays for thyroid cancer.

In one embodiment, the invention pertains to a kit for assaying a sample from a subject to detect a susceptibility to thyroid cancer in the subject, wherein the kit comprises reagents necessary for selectively detecting at least one at-risk variant for thyroid cancer in the individual, wherein the at least one at-risk variant is selected from the group consisting of rs334725, rs28933981 and rs116909374, and markers in linkage disequilibrium therewith. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with thyroid cancer risk. In one such embodiment, the polymorphism is selected from the group consisting of rs334725, rs28933981 and rs116909374, and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking the polymorphism. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In one embodiment, the DNA template is amplified before detection by PCR. The DNA template may also be amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

In certain embodiments, determination of the presence of a particular marker allele (e.g. allele C of rs334725, allele T of rs28933981 and/or allele T of rs116909374) is indicative of a increased susceptibility of thyroid cancer. In another embodiment, determination of the presence of a particular marker allele is indicative of prognosis of thyroid cancer. In another embodiment, the presence of a marker allele is indicative of response to a therapeutic agent for thyroid cancer. In yet another embodiment, the presence of a marker allele is indicative of progress of treatment of thyroid cancer.

In certain embodiments, the kit comprises reagents for detecting no more than 100 alleles in the genome of the individual. In certain other embodiments, the kit comprises reagents for detecting no more than 20 alleles in the genome of the individual.

In a further aspect of the present invention, a pharmaceutical pack (kit) is provided, the pack comprising a therapeutic agent and a set of instructions for administration of the therapeutic agent to humans diagnostically tested for an at-risk variant for thyroid cancer. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention (e.g., an at-risk variant) is instructed to take a prescribed dose of the therapeutic agent. In another embodiment, an individual identified as a non-carrier of at least one variant of the present invention (e.g., an at-risk variant) is instructed to take a prescribed dose of the therapeutic agent.

In certain embodiments, the kit further comprises a set of instructions for using the reagents comprising the kit. In certain embodiments, the kit further comprises a collection of data comprising correlation data between the at least one at-risk variant and susceptibility to thyroid cancer.

### Antisense Agents

The nucleic acids and/or variants described herein, e.g. the rs334725, rs28933981 and rs116909374 variants, or variants in linkage disequilibrium therewith, or nucleic acids comprising their complementary sequence, may be used as antisense constructs to control gene expression in cells, tissues or organs. The methodology associated with antisense techniques is well known to the skilled artisan, and is for example described and reviewed in AntisenseDrug Technology: Principles, Strategies, and Applications, Crooke, ed., Marcel Dekker Inc., New York (2001). In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002).

In certain embodiments, the antisense agent is an oligonucleotide that is capable of binding to a particular nucleotide segment. In certain embodiments, the nucleotide segment is a segment comprising the human TTR gene. In certain embodiments, the nucleotide segment comprises the a marker selected from the group consisting of rs334725, rs28933981 rs116909374, and markers in linkage disequilibrium therewith. In certain embodiments, the nucleotide segment comprises a sequence as set forth in any of SEQ ID NO:1-210. Antisense nucleotides can be from 5-400 nucleotides in length, including 5-200 nucleotides, 5-100 nucleotides, 10-50 nucleotides, and 10-30 nucleotides. In certain preferred embodiments, the antisense nucleotides is from 14-50 nucleotides in length, including 14-40 nucleotides and 14-30 nucleotides.

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention can be inhibited or blocked. In one embodiment, the antisense molecules are designed to specifically bind a particular allelic form of the target nucleic acid, thereby inhibiting translation of a product originating from this specific allele, but which do not bind other or alternate variants at the specific polymorphic sites of the target nucleic acid molecule. In one embodiment, the antisense molecule is designed to specifically bind to nucleic acids comprising the C allele of rs334725, the T allele of rs28933981 and/or the T allele of rs116909374. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988). In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence as set forth in any one of SEQ ID NO:1-210, or a nucleotide sequence comprising, or consisting of, the complement of the nucleotide sequence of any one of SEQ ID NO:1-210. The nucleic acid fragments of the invention are suitably at least about 15, at least about 18, 20, 23 or 25 nucleotides, and can be up to 30, 40, 50, 100, 200, 300 or 400 nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

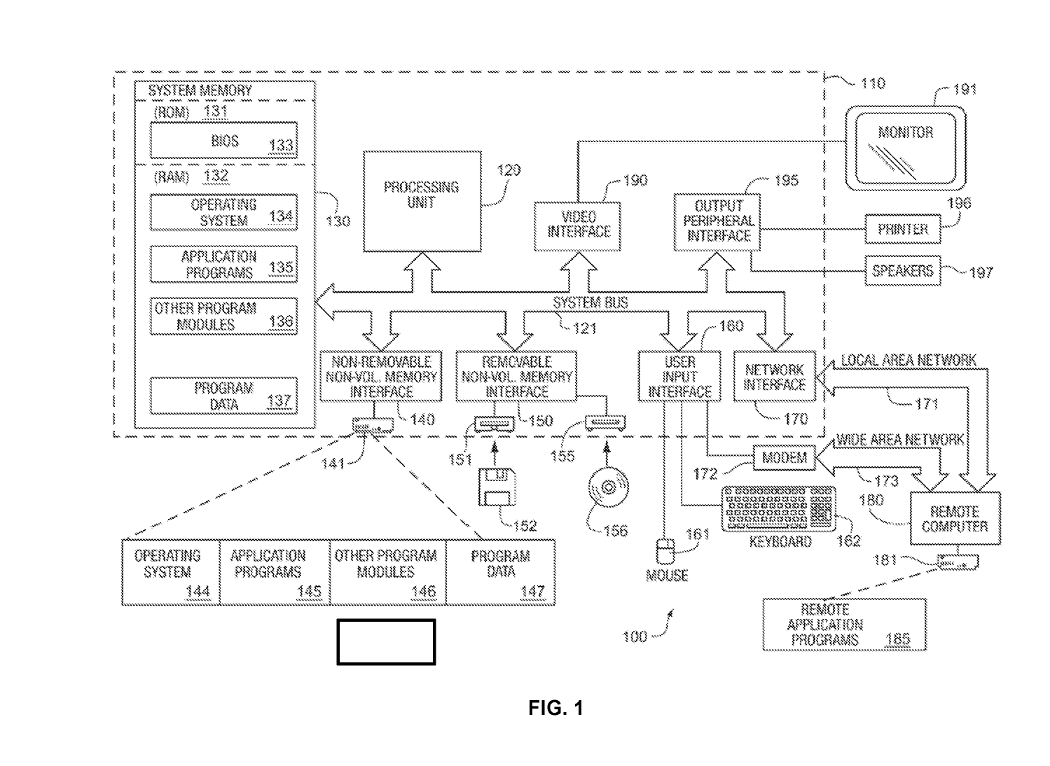

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, certain aspects of the invention relate to computer-implemented applications using the polymorphic markers and haplotypes described herein, and genotype and/or disease-association data derived therefrom. Such applications can be useful for storing, manipulating or otherwise analyzing genotype data that is useful in the methods of the invention. One example pertains to storing genotype and/or sequence data derived from an individual on readable media, so as to be able to provide the data to a third party (e.g., the individual, a guardian of the individual, a health care provider or genetic analysis service provider), or for deriving information from the data, e.g., by comparing the data to information about genetic risk factors contributing to increased susceptibility thyroid cancer, and reporting results based on such comparison.

In certain embodiments, computer-readable media suitably comprise capabilities of storing (i) identifier information for at least one polymorphic marker (e.g, marker names), as described herein; (ii) an indicator of the identity (e.g., presence or absence) of at least one allele of said at least one marker in individuals with thyroid cancer (e.g., rs334725, rs28933981 and/or rs116909374); and (iii) an indicator of the risk associated with a particular marker allele (e.g., the C allele of rs334725, the T allele of rs28933981 and/or the T allele of rs116909374). The media may also suitably comprise capabilities of storing protein sequence data.

In one embodiment, the invention provides a computer-readable medium having computer executable instructions for determining susceptibility to thyroid cancer in a human individual, the computer readable medium comprising (i) sequence data identifying at least one allele of at least one polymorphic marker in the individual; and (ii) a routine stored on the computer readable medium and adapted to be executed by a processor to determine risk of developing thyroid cancer for the at least one polymorphic marker; wherein the at least one polymorphic marker is selected from the group consisting of rs334725, rs28933981 and rs116909374, and markers in linkage disequilibrium therewith. In certain embodiments, markers in linkage disequililbrium with rs334725 are selected from the markers listed in Tables 1 and 7 herein. In certain embodiments, markers in linkage disequilibrium with rs116909374 are selected from the markers listed in Tables 2 and 8 herein. In one embodiment, the at least one polymorphic marker is rs334725. In another embodiment, the at least one polymorphism is rs116909374. In another embodiment, the at least one polymorphism is rs28933981.

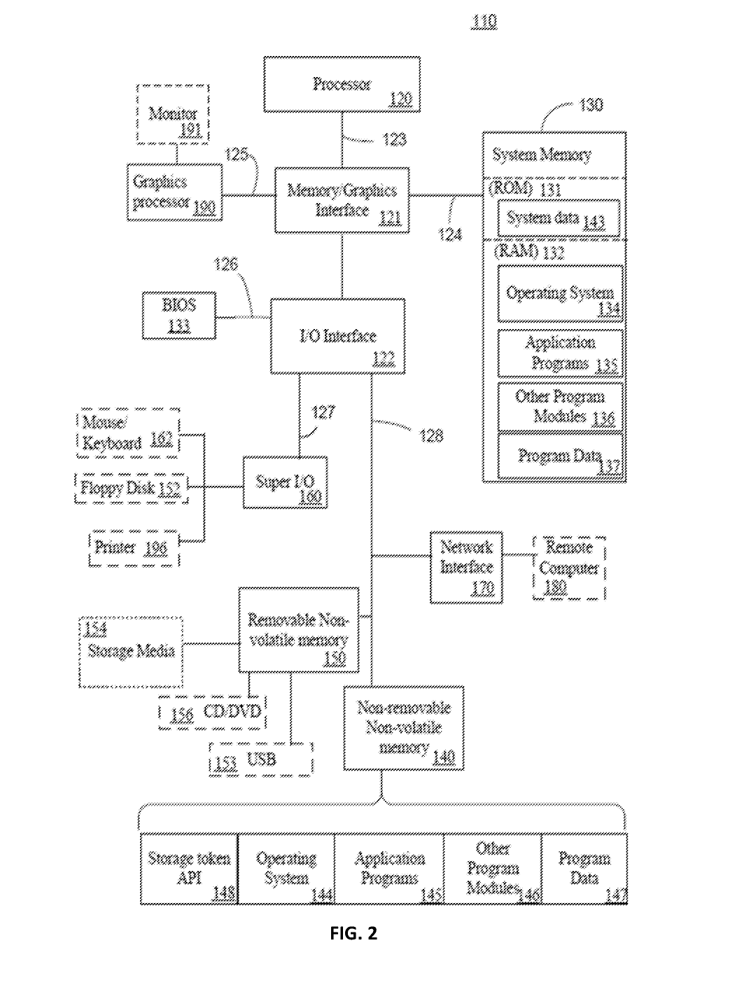

With reference to FIG. 2, a second exemplary system of the invention, which may be used to implement one or more steps of methods of the invention, includes a computing device in the form of a computer 110. Components shown in dashed outline are not technically part of the computer 110, but are used to illustrate the exemplary embodiment of FIG. 2. Components of computer 110 may include, but are not limited to, a processor 120, a system memory 130, a memory/graphics interface 121, also known as a Northbridge chip, and an I/O interface 122, also known as a Southbridge chip. The system memory 130 and a graphics processor 190 may be coupled to the memory/graphics interface 121. A monitor 191 or other graphic output device may be coupled to the graphics processor 190.

A series of system busses may couple various system components including a high speed system bus 123 between the processor 120, the memory/graphics interface 121 and the I/O interface 122, a front-side bus 124 between the memory/graphics interface 121 and the system memory 130, and an advanced graphics processing (AGP) bus 125 between the memory/graphics interface 121 and the graphics processor 190. The system bus 123 may be any of several types of bus structures including, by way of example, and not limitation, such architectures include Industry Standard Architecture (USA) bus, Micro Channel Architecture (MCA) bus and Enhanced ISA (EISA) bus. As system architectures evolve, other bus architectures and chip sets may be used but often generally follow this pattern. For example, companies such as Intel and AMD support the Intel Hub Architecture (INA) and the Hypertransport™ architecture, respectively.

The computer 110 typically includes a variety of computer-readable media. Computer-readable media can be any available media that can be accessed by computer 110 and includes both volatile and nonvolatile media, removable and non-removable media. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other physical medium which can be used to store the desired information and which can accessed by computer 110.

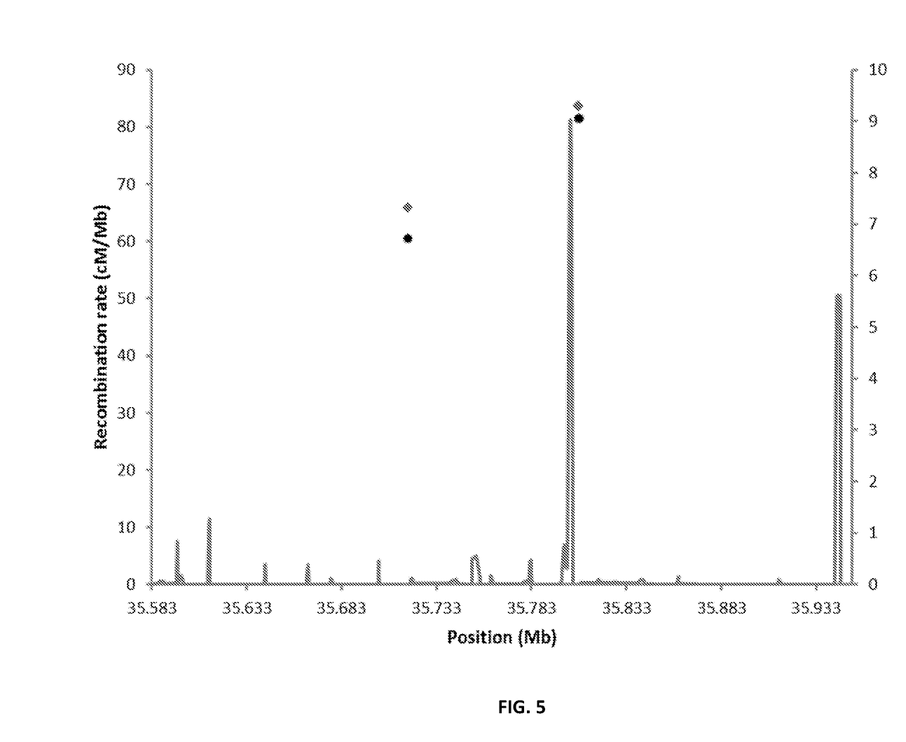

The system memory 130 includes computer storage media in the form of volatile and/or nonvolatile memory such as read only memory (ROM) 131 and random access memory (RAM) 132. The system ROM 131 may contain permanent system data 143, such as identifying and manufacturing information. In some embodiments, a basic input/output system (BIOS) may also be stored in system ROM 131. RAM 132 typically contains data and/or program modules that are immediately accessible to and/or presently being operated on by processor 120. By way of example, and not limitation, FIG. 5 illustrates operating system 134, application programs 135, other program modules 136, and program data 137.

The I/O interface 122 may couple the system bus 123 with a number of other busses 126, 127 and 128 that couple a variety of internal and external devices to the computer 110. A serial peripheral interface (SPI) bus 126 may connect to a basic input/output system (BIOS) memory 133 containing the basic routines that help to transfer information between elements within computer 110, such as during start-up.

A super input/output chip 160 may be used to connect to a number of ‘legacy’ peripherals, such as floppy disk 152, keyboard/mouse 162, and printer 196, as examples. The super I/O chip 160 may be connected to the I/O interface 122 with a bus 127, such as a low pin count (LPC) bus, in some embodiments. Various embodiments of the super I/O chip 160 are widely available in the commercial marketplace.

In one embodiment, bus 128 may be a Peripheral Component Interconnect (PCI) bus, or a variation thereof, may be used to connect higher speed peripherals to the I/O interface 122. A PCI bus may also be known as a Mezzanine bus. Variations of the PCI bus include the Peripheral Component Interconnect-Express (PCI-E) and the Peripheral Component Interconnect-Extended (PCI-X) busses, the former having a serial interface and the latter being a backward compatible parallel interface. In other embodiments, bus 128 may be an advanced technology attachment (ATA) bus, in the form of a serial ATA bus (SATA) or parallel ATA (PATA).

The computer 110 may also include other removable/non-removable, volatile/nonvolatile computer storage media. By way of example only, FIG. 2 illustrates a hard disk drive 140 that reads from or writes to non-removable, nonvolatile magnetic media. The hard disk drive 140 may be a conventional hard disk drive.

Removable media, such as a universal serial bus (USB) memory 153, firewire (IEEE 1394), or CD/DVD drive 156 may be connected to the PCI bus 128 directly or through an interface 150. A storage media 154 may coupled through interface 150. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

The drives and their associated computer storage media discussed above and illustrated in FIG. 2, provide storage of computer readable instructions, data structures, program modules and other data for the computer 110. In FIG. 2, for example, hard disk drive 140 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a mouse/keyboard 162 or other input device combination. Other input devices (not shown) may include a microphone, joystick, game pad, satellite dish, scanner, or the like. These and other input devices are often connected to the processor 120 through one of the I/O interface busses, such as the SPI 126, the LPC 127, or the PCI-128, but other busses may be used. In some embodiments, other devices may be coupled to parallel ports, infrared interfaces, game ports, and the like (not depicted), via the super I/O chip 160.

The computer 110 may operate in a networked environment using logical connections to one or more remote computers, such as a remote computer 180 via a network interface controller (NIC) 170. The remote computer 180 may be a personal computer, a server, a router, a network PC, a peer device or other common network node, and typically includes many or all of the elements described above relative to the computer 110. The logical connection between the NIC 170 and the remote computer 180 depicted in FIG. 2 may include a local area network (LAN), a wide area network (WAN), or both, but may also include other networks. Such networking environments are commonplace in offices, enterprise-wide computer networks, intranets, and the Internet. The remote computer 180 may also represent a web server supporting interactive sessions with the computer 110, or in the specific case of location-based applications may be a location server or an application server.

In some embodiments, the network interface may use a modem (not depicted) when a broadband connection is not available or is not used. It will be appreciated that the network connection shown is exemplary and other means of establishing a communications link between the computers may be used.

In some variations, the invention is a system for identifying susceptibility to thyroid cancer in a human subject. For example, in one variation, the system includes tools for performing at least one step, preferably two or more steps, and in some aspects all steps of a method of the invention, where the tools are operably linked to each other. Operable linkage describes a linkage through which components can function with each other to perform their purpose.

In some variations, a system of the invention is a system for identifying susceptibility to thyroid cancer in a human subject, and comprises:
(a) at least one processor;
(b) at least one computer-readable medium;
(c) a susceptibility database operatively coupled to a computer-readable medium of the system and containing population information correlating the presence or absence of one or more alleles of a marker selected from the group consisting of rs334725, rs28933981 and rs116909374, and markers in linkage disequilibrium therewith and susceptibility to thyroid cancer in a population of humans;
(d) a measurement tool that receives an input about the human subject and generates information from the input about the presence or absence of the at least one allele in the human subject; and
(e) an analysis tool or routine that:
   (i) is operatively coupled to the susceptibility database and the information generated by the measurement tool,
   (ii) is stored on a computer-readable medium of the system,
   (iii) is adapted to be executed on a processor of the system, to compare the information about the human subject with the population information in the susceptibility database and generate a conclusion with respect to susceptibility to thyroid cancer for the human subject.

Exemplary processors (processing units) include all variety of microprocessors and other processing units used in computing devices. Exemplary computer-readable media are described above. When two or more components of the system involve a processor or a computer-readable medium, the system generally can be created where a single processor and/or computer readable medium is dedicated to a single component of the system; or where two or more functions share a single processor and/or share a single computer readable medium, such that the system contains as few as one processor and/or one computer readable medium. In some variations, it is advantageous to use multiple processors or media, for example, where it is convenient to have components of the system at different locations. For instance, some components of a system may be located at a testing laboratory dedicated to laboratory or data analysis, whereas other components, including components (optional) for supplying input information or obtaining an output communication, may be located at a medical treatment or counseling facility (e.g., doctor's office, health clinic, HMO, pharmacist, geneticist, hospital) and/or at the home or business of the human subject (patient) for whom the testing service is performed.

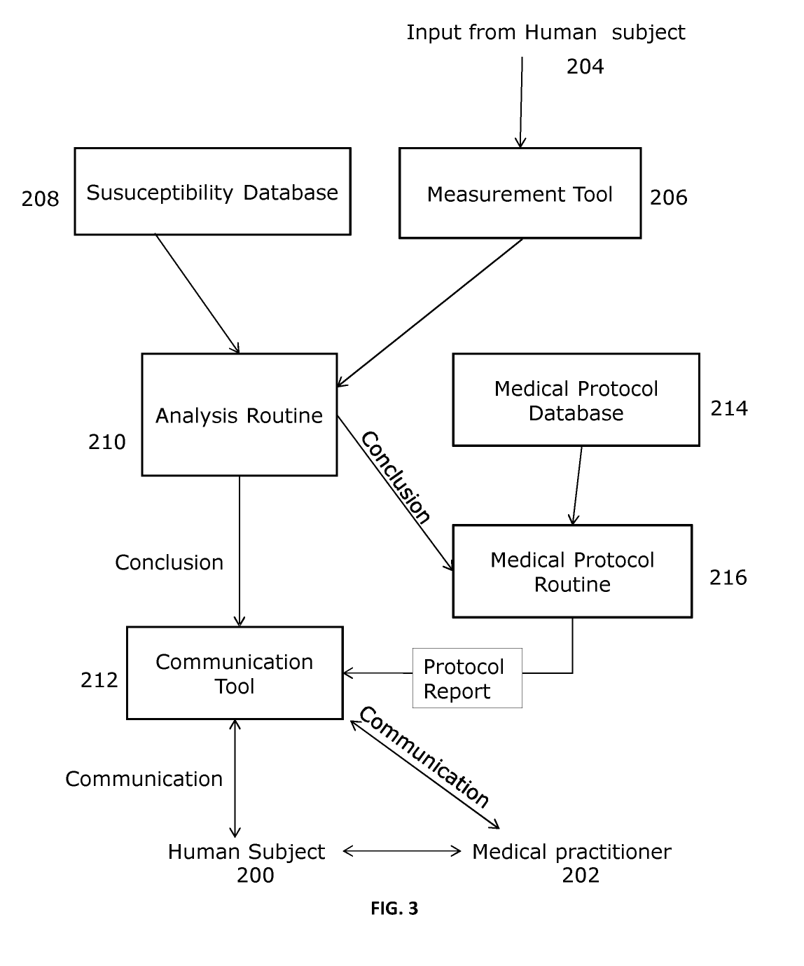

Referring to FIG. 3, an exemplary system includes a susceptibility database 208 that is operatively coupled to a computer-readable medium of the system and that contains population information correlating the presence or absence of one or more alleles of a polymorphic marker selected from rs334725, rs28933981 and rs116909374, and markers in linkage disequilibrium therewith and susceptibility to thyroid cancer in a population of humans.

In certain embodiments, markers in linkage disequililbrium with rs334725 are selected from the markers listed in Tables 1 and 7 herein. In certain embodiments, markers in linkage disequilibrium with rs116909374 are selected from the markers listed in Tables 2 and 8 herein.

In a simple variation, the susceptibility database contains 208 data relating to the frequency that a particular marker allele selected from the group has been observed in a population of humans with thyroid cancer and a population of humans free of thyroid cancer. Such data provides an indication as to the relative risk or odds ratio of developing thyroid cancer for a human subject that is identified as having the allele in question. In another variation, the susceptibility database includes similar data with respect to two or more markers, thereby providing a useful reference if the human subject has any of the two or more alleles of the two or more markers. In still another variation, the susceptibility database includes additional quantitative personal, medical, or genetic information about the individuals in the database diagnosed with thyroid cancer or free of thyroid cancer. Such information includes, but is not limited to, information about parameters such as age, sex, ethnicity, race, medical history, weight, diabetes status, blood pressure, family history of thyroid cancer, smoking history, and alcohol use in humans and impact of the at least one parameter on susceptibility to thyroid cancer. The information also can include information about other genetic risk factors for thyroid cancer besides the genetic variants described herein. These more robust susceptibility databases can be used by an analysis routine 210 to calculate a combined score with respect to susceptibility or risk for developing thyroid cancer.

In addition to the susceptibility database 208, the system further includes a measurement tool 206 programmed to receive an input 204 from or about the human subject and generate an output that contains information about the presence or absence of the at least one marker allele of interest. (The input 204 is not part of the system per se but is illustrated in the schematic FIG. 3.) Thus, the input 204 will contain a specimen or contain data from which the presence or absence of the at least one marker allele can be directly read, or analytically determined. In a simple variation, the input contains annotated information about genotypes or allele counts for particular markers such as rs334725, rs28933981 and rs116909374, and markers in linkage disequilibrium therewith, in the genome of the human subject, in which case no further processing by the measurement tool 206 is required, except possibly transformation of the relevant information about the presence/absence of the at least one marker allele into a format compatible for use by the analysis routine 210 of the system.

In another variation, the input 204 from the human subject contains data that is unannotated or insufficiently annotated with respect to risk markers for thyroid cancer selected from rs334725, rs28933981 and rs116909374, and markers in linkage disequilibrium therewith, requiring analysis by the measurement tool 206. For example, the input can be genetic sequence of the chromosomal region or chromosome on which the markers reside, or whole genome sequence information, or unannotated information from a gene chip analysis of a variable loci in the human subject's genome. In such variations of the invention, the measurement tool 206 comprises a tool, preferably stored on a computer-readable medium of the system and adapted to be executed on a processor of the system, to receive a data input about a subject and determine information about the presence or absence of the at least one marker allele in a human subject from the data. For example, the measurement tool 206 contains instructions, preferably executable on a processor of the system, for analyzing the unannotated input data and determining the presence or absence of the marker allele of interest in the human subject. Where the input data is genomic sequence information, and the measurement tool optionally comprises a sequence analysis tool stored on a computer readable medium of the system and executable by a processor of the system with instructions for determining the presence or absence of the at least one mutant marker allele from the genomic sequence information.

In yet another variation, the input 204 from the human subject comprises a biological sample, such as a fluid (e.g., blood) or tissue sample that contains genetic material that can be analyzed to determine the presence or absence of particular marker allele(s) of interest. In this variation, an exemplary measurement tool 206 includes laboratory equipment for processing and analyzing the sample to determine the presence or absence (or identity) of the marker allele(s) in the human subject. For instance, in one variation, the measurement tool includes: an oligonucleotide microarray (e.g., “gene chip”) containing a plurality of oligonucleotide probes attached to a solid support; a detector for measuring interaction between nucleic acid obtained from or amplified from the biological sample and one or more oligonucleotides on the oligonucleotide microarray to generate detection data; and an analysis tool stored on a computer-readable medium of the system and adapted to be executed on a processor of the system, to determine the presence or absence of the at least one marker allele of interest based on the detection data.

To provide another example, in some variations the measurement tool 206 includes: a nucleotide sequencer (e.g., an automated DNA sequencer) that is capable of determining nucleotide sequence information from nucleic acid obtained from or amplified from the biological sample; and an analysis tool stored on a computer-readable medium of the system and adapted to be executed on a processor of the system, to determine the presence or absence of the at least one marker allele based on the nucleotide sequence information.

In some variations, the measurement tool 206 further includes additional equipment and/or chemical reagents for processing the biological sample to purify and/or amplify nucleic acid of the human subject for further analysis using a sequencer, gene chip, or other analytical equipment.

The exemplary system further includes an analysis tool or routine 210 that: is operatively coupled to the susceptibility database 208 and operatively coupled to the measurement tool 206, is stored on a computer-readable medium of the system, is adapted to be executed on a processor of the system to compare the information about the human subject with the population information in the susceptibility database 208 and generate a conclusion with respect to susceptibility to thyroid cancer for the human subject. In simple terms, the analysis tool 210 looks at the marker alleles identified by the measurement tool 206 for the human subject, and compares this information to the susceptibility database 208, to determine a susceptibility to thyroid cancer for the subject. The susceptibility can be based on the single parameter (the identity of one or more marker alleles), or can involve a calculation based on other genetic and non-genetic data, as described above, that is collected and included as part of the input 204 from the human subject, and that also is stored in the susceptibility database 208 with respect to a population of other humans. Generally speaking, each parameter of interest is weighted to provide a conclusion with respect to susceptibility to thyroid cancer. Such a conclusion is expressed in the conclusion in any statistically useful form, for example, as an odds ratio, a relative risk, or a lifetime risk for subject developing thyroid cancer.

In some variations of the invention, the system as just described further includes a communication tool 212. For example, the communication tool is operatively connected to the analysis routine 210 and comprises a routine stored on a computer-readable medium of the system and adapted to be executed on a processor of the system, to: generate a communication containing the conclusion; and to transmit the communication to the human subject 200 or the medical practitioner 202, and/or enable the subject or medical practitioner to access the communication. (The subject and medical practitioner are depicted in the schematic FIG. 3, but are not part of the system per se, though they may be considered users of the system. The communication tool 212 provides an interface for communicating to the subject, or to a medical practitioner for the subject (e.g., doctor, nurse, genetic counselor), the conclusion generated by the analysis tool 210 with respect to susceptibility to thyroid cancer for the subject. Usually, if the communication is obtained by or delivered to the medical practitioner 202, the medical practitioner will share the communication with the human subject 200 and/or counsel the human subject about the medical significance of the communication. In some variations, the communication is provided in a tangible form, such as a printed report or report stored on a computer readable medium such as a flash drive or optical disk. In some variations, the communication is provided electronically with an output that is visible on a video display or audio output (e.g., speaker). In some variations, the communication is transmitted to the subject or the medical practitioner, e.g., electronically or through the mail. In some variations, the system is designed to permit the subject or medical practitioner to access the communication, e.g., by telephone or computer. For instance, the system may include software residing on a memory and executed by a processor of a computer used by the human subject or the medical practitioner, with which the subject or practitioner can access the communication, preferably securely, over the internet or other network connection. In some variations of the system, this computer will be located remotely from other components of the system, e.g., at a location of the human subject's or medical practitioner's choosing.

In some variations of the invention, the system as described (including embodiments with or without the communication tool) further includes components that add a treatment or prophylaxis utility to the system. For instance, value is added to a determination of susceptibility to thyroid cancer when a medical practitioner can prescribe or administer a standard of care that can reduce susceptibility to thyroid cancer; and/or delay onset of thyroid cancer; and/or increase the likelihood of detecting the cancer at an early stage. Exemplary lifestyle change protocols include loss of weight, increase in exercise, cessation of unhealthy behaviors such as smoking, and change of diet. Exemplary medicinal and surgical intervention protocols include administration of pharmaceutical agents for prophylaxis; and surgery.

For example, in some variations, the system further includes a medical protocol database 214 operatively connected to a computer-readable medium of the system and containing information correlating the presence or absence of the at least one marker allele of interest and medical protocols for human subjects at risk for the cancer. Such medical protocols include any variety of medicines, lifestyle changes, diagnostic tests, increased frequencies of diagnostic tests, and the like that are designed to achieve one of the aforementioned goals. The information correlating a marker allele with protocols could include, for example, information about the success with which the cancer is avoided or delayed, or success with which the cancer is detected early and treated, if a subject has a particular susceptibility allele and follows a protocol.

The system of this embodiment further includes a medical protocol tool or routine 216, operatively connected to the medical protocol database 214 and to the analysis tool or routine 210. The medical protocol tool or routine 216 preferably is stored on a computer-readable medium of the system, and adapted to be executed on a processor of the system, to: (i) compare (or correlate) the conclusion that is obtained from the analysis routine 210 (with respect to susceptibility to thyroid cancer for the subject) and the medical protocol database 214, and (ii) generate a protocol report with respect to the probability that one or more medical protocols in the medical protocol database will achieve one or more of the goals of reducing susceptibility to the cancer; delaying onset of the cancer; and increasing the likelihood of detecting the cancer at an early stage to facilitate early treatment. The probability can be based on empirical evidence collected from a population of humans and expressed either in absolute terms (e.g., compared to making no intervention), or expressed in relative terms, to highlight the comparative or additive benefits of two or more protocols.

Some variations of the system include the communication tool 212. In some examples, the communication tool generates a communication that includes the protocol report in addition to, or instead of, the conclusion with respect to susceptibility.

Information about marker allele status not only can provide useful information about identifying or quantifying susceptibility to thyroid cancer; it can also provide useful information about possible causative factors for a human subject identified with thyroid cancer, and useful information about therapies for the patient. In some variations, systems of the invention are useful for these purposes.

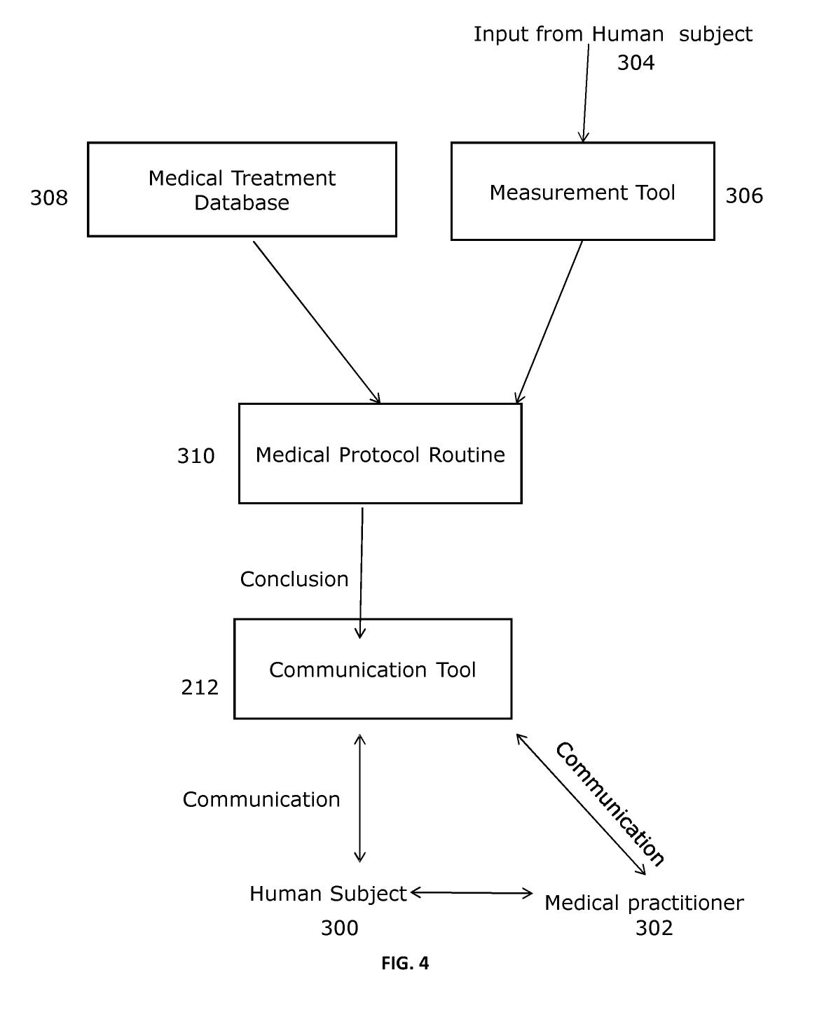

For instance, in some variations the invention is a system for assessing or selecting a treatment protocol for a subject diagnosed with thyroid cancer. An exemplary system, schematically depicted in FIG. 4, comprises:
(a) at least one processor;
(b) at least one computer-readable medium;
(c) a medical treatment database 308 operatively connected to a computer-readable medium of the system and containing information correlating the presence or absence of at least one allele of a marker selected from the group consisting of rs334725, rs28933981 and rs116909374, and markers in linkage disequilibrium therewith and efficacy of treatment regimens for thyroid cancer;
(d) a measurement tool 306 to receive an input (304, depicted in FIG. 4 but not part of the system per se) about the human subject and generate information from the input 304 about the presence or absence of the at least one marker allele in a human subject diagnosed with thyroid cancer; and
(e) a medical protocol routine or tool 310 operatively coupled to the medical treatment database 308 and the measurement tool 306, stored on a computer-readable medium of the system, and adapted to be executed on a processor of the system, to compare the information with respect to presence or absence of the at least one marker allele for the subject and the medical treatment database, and generate a conclusion with respect to at least one of:
   (i) the probability that one or more medical treatments will be efficacious for treatment of thyroid cancer for the patient; and
   (ii) which of two or more medical treatments for thyroid cancer will be more efficacious for the patient.

Preferably, such a system further includes a communication tool 312 operatively connected to the medical protocol tool or routine 310 for communicating the conclusion to the subject 300, or to a medical practitioner for the subject 302 (both depicted in the schematic of FIG. 4, but not part of the system per se). An exemplary communication tool comprises a routine stored on a computer-readable medium of the system and adapted to be executed on a processor of the system, to generate a communication containing the conclusion; and transmit the communication to the subject or the medical practitioner, or enable the subject or medical practitioner to access the communication.

In a further embodiment, the invention provides a computer-readable medium having computer executable instructions for determining susceptibility to thyroid cancer in a human individual, the computer readable medium comprising (i) sequence data identifying at least one allele of at least one polymorphic marker in the individual; and (ii) a routine stored on the computer readable medium and adapted to be executed by a processor to determine risk of developing thyroid cancer for the at least one polymorphic marker; wherein the at least one polymorphic marker is a marker selected from the group consisting of rs334725, rs28933981 and rs116909374, and markers in linkage disequilibrium therewith, that is predictive of susceptibility of thyroid cancer in humans. In one embodiment, the at least one polymorphic marker is selected from the group consisting of rs116909374, and markers in linkage disequilibrium therewith. In certain embodiments, markers in linkage disequililbrium with rs334725 are selected from the markers listed in Tables 1 and 7 herein. In certain embodiments, markers in linkage disequilibrium with rs116909374 are selected from the markers listed in Tables 2 and 8 herein. In one preferred embodiment, the polymorphic marker is rs116909374.

In certain embodiments, a report is prepared, which contains results of a determination of susceptibility of thyroid cancer. The report may suitably be written in any computer readable medium, printed on paper, or displayed on a visual display.

The present invention will now be exemplified by the following non-limiting examples.

### Example 1

Association of markers on chromosome 1p31.3 (rs334725), chromosome 14q13.3 (rs116909374) and chromosome 18q12.1 (rs28933981) with thyroid cancer was investigated. The chromosome 1p31 and 14q13 markers were previously found to be associated with levels of thyroid stimulating hormone (TSH), and the chromosome 18q12 marker with levels of free thyroxin (T4), leading to the speculation that these markers might also be associated with risk of thyroid cancer.

### Subjects

Approval for this study was granted by the National Bioethics Committee of Iceland and the Icelandic Data Protection Authority.

Our collection of samples used for the thyroid cancer study represents the overall distribution in Iceland quite well. Of the cases that we generated genotypes for either by directly genotyping or in-silico genotyping, about 80% are of papillary type, about 12% are of follicular type, about 2% are medullary thyroid cancer, and the remainders are of unknown or undetermined histological sub-phenotype.

The results presented in Table 3 below are for the combined results for all our cases since no statistically significant difference was observed between the different histological subgroups.

The Icelandic controls consist of up to 37,668 individuals from other ongoing genome-wide association studies at deCODE genetics. Individuals with a diagnosis of thyroid cancer were excluded. Both male and female genders were included.

### Genotyping

Markers in Table 3 were genotyped by Centaurus SNP genotyping (Kutyavin, et al., (2006), Nucleic Acids Res, 34, e128) or the Illumnina HumanHap317K SNP chip platform. Genotyping was carried out at the deCODE genetics facility.

### Imputation Analysis

We imputed genotypes for un-genotyped cases of genotyped individuals. For every un-genotyped case, it is possible to calculate the probability of the genotypes of its relatives given its four possible phased genotypes. In practice it may be preferable to include only the genotypes of the case's parents, children, siblings, half-siblings (and the half-sibling's parents), grand-parents, grand-children (and the grand-children's parents) and spouses. It will be assumed that the individuals in the small sub-pedigrees created around each case are not related through any path not included in the pedigree. It is also assumed that alleles that are not transmitted to the case have the same frequency—the population allele frequency. Let us consider a SNP marker with the alleles A and G. The probability of the genotypes of the case's relatives can then be computed by:

$$Pr ( genotypes of relatives ; θ ) = {\sum }_{h ∈ \{ AA , AG , GA , GG \}} Pr ( h ; θ ) Pr ( genotypes of relatives \vert h ) ,$$

where θ denotes the A allele's frequency in the cases. Assuming the genotypes of each set of relatives are independent, this allows us to write down a likelihood function for θ:

$$L ( θ ) = {\prod }_{i} Pr ( genotypes of relatives of case i ; θ ) . \qquad (* )$$

This assumption of independence is usually not correct. Accounting for the dependence between individuals is a difficult and potentially prohibitively expensive computational task. The likelihood function in (*) may be thought of as a pseudolikelihood approximation of the full likelihood function for θ which properly accounts for all dependencies. In general, the genotyped cases and controls in a case-control association study are not independent and applying the case-control method to related cases and controls is an analogous approximation. The method of genomic control (Devlin, B. et al., Nat Genet 36, 1129-30; author reply 1131 (2004)) has proven to be successful at adjusting case-control test statistics for relatedness. We therefore apply the method of genomic control to account for the dependence between the terms in our pseudolikelihood and produce a valid test statistic.

Fisher's information can be used to estimate the effective sample size of the part of the pseudolikelihood due to un-genotyped cases. Breaking the total Fisher information, I, into the part due to genotyped cases, I_{g}, and the part due to ungenotyped cases, Iᵤ, I=I_{g}+Iᵤ, and denoting the number of genotyped cases with N, the effective sample size due to the un-genotyped cases is estimated by

$$\frac{{I}_{u}}{{I}_{g}} N .$$

Data for rs334725 and rs28933981 were generated using Centaurus assay for genotyping samples from 558 Icelandic individuals with thyroid cancer, and genotypes for 38,764 Icelandic population controls were determined using the Illumina HumanHap317K SNP chip. Data for rs116909374 were generated using Centaurus assay for genotyping samples from 542 Icelandic individuals with thyroid cancer and 1,518 Icelandic control individuals.

Results of association analysis is shown below in Table 3. As can be seen, the markers rs334725 and rs116909374 are found to be significantly associated with thyroid cancer, with risk more than 1.3 and 1.8, respectively. The observed risk for rs28933981 is even higher, at 2.8.

TABLE 3
Association of markers rs334725, rs116909374
and rs28933981 with Thyroid cancer.

| | | | | freq | freq | | |
|---|---|---|---|---|---|---|---|
| Marker | Chr | Pos (Build 36) | allele | cases | ctrls | OR | p-value |
| rs334725 | 1p31.3 | 61,382,637 | C | 0.0851 | 0.0652 | 1.3346 | 0.0103 |
| rs116909374 | 14q13.3 | 35,808,112 | T | 0.0849 | 0.0474 | 1.8626 | 1.19 × 10⁻⁵ |
| rs28933981 | 18q12.1 | 27,432,508 | T | 0.00682 | 0.0024 | 2.82 | 0.0583 |

### Example 2

A follow-up study of the association of rs116909374 with thyroid cancer was conducted in three case-control groups of European descent, with populations from Ohio, United States (US) the Netherlands and Spain. Data for the association in Iceland was also supplemented by additional controls.

### Study Populations

The Netherlands.

The Dutch study population consists of 151 non-medullary thyroid cancer cases (75% are females) and 832 cancer-free individuals (54% females). The cases were recruited from the Department of Endocrinology, Radboud University Nijmegen Medical Centre (RUNMC), Nijmegen, The Netherlands from November 2009 to June 2010. All patients were of self-reported European descent. Demographic, clinical, tumor treatment and follow-up related characteristics were obtained from the patient's medical records. The average age at diagnosis for the patients was 39 years (SD 12.8). The DNA for both the Dutch cases and controls was isolated from whole blood using standard methods. The controls were recruited within a project entitled “Nijmegen Biomedical Study” (NBS). The details of this study have been reported previously (Wetzels, J. F et al. Kidney Int 72, (2007)). Control individuals from the NBS were invited to participate in a study on gene-environment interactions in multifactorial diseases such as cancer. They were all of self-reported European descent and fully informed about the goals and the procedures of the study. The study was approved by the Ethical Committee and the Institutional Review Board of the RUNMC, Nijmegen, The Netherlands and all study subjects gave written informed consent.

Ohio, USA.

The study was approved by the Institutional Review Board of the Ohio State University. All subjects were of self-reported European descent and provided written informed consent. These patients (n=365; median age 40 years, range 13 to 80; 76% are females) were recruited from Ohio, US and were histologically confirmed papillary thyroid carcinoma (PTC) patients (including traditional PTC and follicular variant PTC). Controls (n=383; median age 49 years, range 18 to 87; 65% are females) were individuals without clinically diagnosed thyroid cancer from the central Ohio area. Genomic DNA was extracted from blood.

Zaragoza, Spain.

The Spanish study population consisted of 90 non-medullary thyroid cancer cases. The cases were recruited from the Oncology Department of Zaragoza Hospital in Zaragoza, Spain, from October 2006 to June 2007. All patients were of self-reported European descent. Clinical information including age at onset, grade and stage was obtained from medical records. The average age at diagnosis for the patients was 48 years (median 49 years) and the range was from 22 to 79 years. The 1,399 Spanish control individuals 798 (57%) males and 601 (43%) females had a mean age of 51 (median age 50 and range 12-87 years) were approached at the University Hospital in Zaragoza, Spain, and were not known to have thyroid cancer. The DNA for both the Spanish cases and controls was isolated from whole blood using standard methods. Study protocols were approved by the Institutional Review Board of Zaragoza University Hospital. All subjects gave written informed consent. Combining the results from Iceland and the follow-up groups gave OR estimates of 2.09 and a P value of 4.6×10⁻¹¹ (see Table 4).

TABLE 4
Association results for rs116909374-T on 14q13.3 and Thyroid cancer
in Iceland, the Netherlands, the United States and Spain

| Study population | | | | | |
|---|---|---|---|---|---|
| (n cases/ | | | | Case | Controls |
| n controls) | OR | 95% CI | P-value | (freq) | (freq) |
| Iceland | 2.03 | (1.54, 2.67) | 5.4 × 10⁻⁷ | 0.085 | 0.044 |
| (542/3,190) | | | | | |
| The Netherlands | 1.95 | (1.09, 3.48) | 0.024 | 0.056 | 0.030 |
| (151/824) | | | | | |
| Ohio, US | 1.98 | (1.12, 3.49) | 0.018 | 0.049 | 0.025 |
| (356/374) | | | | | |
| Spain | 3.37 | (1.53, 7.44) | 2.6 × 10⁻³ | 0.056 | 0.017 |
| (89/952) | | | | | |
| All combined | 2.09 | (1.68, 2.60) | 4.6 × 10⁻¹¹ | | |
| (1,138/5,340) | | | | | |
| Pₕₑₜ | | 0.67 | | | |
| I² | | 0.0 | | | |

Shown are the results for SNPs directly genotyped using single-track assay in cases and controls (n), allelic frequencies of risk variants in affected and control individuals, the allelic odds ratio (OR) with 95% confidence interval (95% CI) and P values based on the multiplicative model. All P values shown are two-sided. For the combined study populations, the OR and the P value were estimated using the Mantel-Haenszel model.

### Example 3

The rs116909374 variant and a previously reported thyroid associated variant rs944289, are located within two distinct but neighboring LD-regions (FIG. 5). The correlation between the markers is very low (r²=0.005, D′=0.35, according to data from 3,693 Icelanders) and the association with thyroid cancer for each SNP remains significant after adjusting for the other (Table 5). This means that the two markers are most likely capturing independent association signals on chromosome 14q13.3.

TABLE 5
Association results for rs116909374 and rs944289
on 14q13.3, before and after adjustment

| | |
|---|---|
| rs116909374-T | rs944289-T |

| Study group | OR | P-value | OR | P-value |
|---|---|---|---|---|
| Iceland | | | | |
| Unadjusted | 2.03 | 5.4E−07 | 1.36 | 4.2E−05 |
| Adjusted | 1.95 | 4.7E−07 | 1.30 | 9.6E−05 |
| The Netherlands | | | | |
| Unadjusted | 1.95 | 0.024 | 1.39 | 0.013 |
| Adjusted | 1.93 | 0.028 | 1.38 | 0.014 |
| Ohio ^{a} | | | | |
| Unadjusted | 1.60 | 0.26 | 1.51 | 0.0067 |
| Adjusted | 1.52 | 0.32 | 1.50 | 0.0078 |
| Spain | | | | |
| Unadjusted | 3.37 | 0.0026 | 1.17 | 0.31 |
| Adjusted | 3.27 | 0.0040 | 1.13 | 0.45 |
| All combined | | | | |
| Unadjusted | 2.07 | 5.0 × 10⁻¹⁰ | 1.36 | 4.9 × 10⁻⁸ |
| Adjusted | 1.99 | 8.7 × 10⁻¹⁰ | 1.32 | 1.9 × 10⁻⁷ |

Shown are results for rs116909374 before and after being adjusted for rs944289 as well as results for rs944289 before and after being adjusted for rs116909374. The two SNPs are only correlated to a very small degree (D′ = 0.35 and r² = 0.005 based on results from 3,693 Icelanders). Results are only presented for individuals where data is available for both SNPs. Pₕₑₜ is >0.5 for both markers.
^{a} For the Ohio samples data was available for both SNPs for 155 cases and 245 controls.
The LD- and correlation information the two SNPs in this table in the four different study groups is as follows:
Iceland; D′ = 0.35 r² = 0.0050
The Netherlands D′ = 0.13 r² = 0.0003
Ohio; D′ = 0.37 r² = 0.0026
Spain; D′ = 0.63 r² = 0.0065

This notion is further supported by the fact that the association effect for Thyroid Stimulating

Hormone (TSH) levels is substantially stronger rs116909374 than for the previously reported rs944289 (effect =−0.141 standard deviation (s.d.) and P=1.1×10⁻¹⁶ for rs116909374 allele T compared to an effect=−0.022 s.d. and P=0.001 for rs944289 allele T). This results suggests that the 14q13.3 locus contains more than one variant predisposing to thyroid cancer or, possibly, that a unique variant capturing the effect of rs116909374 and rs944289 remains to be discovered.

### Example 4

High capacity DNA sequencing techniques were used to sequence the entire genomes of about 1900 Icelanders to an average depth of 10×-30× fold. This identified over 30 million SNPs and Indels. Using imputation assisted by long-range haplotype phasing, sequence data was used to determine the genotypes of the 30 million SNPs in the 71,743 Icelanders who had been genotyped on the SNP chips. Imputation was performed using one or more of four sources, the HapMap2 CEU sample (Nature 437, 1299-320 (2005)) (60 triads), the 1000 Genomes data (Durbin, R. M. et al. Nature 467, 1061-73) (179 individuals) and Icelandic samples genotyped with the Illumine Human1M-Duo and the HumanOmni1-Quad chips. Imputations were based on the IMPUTE model (Marchini, J., Howie, B., Myers, S., McVean, G. & Donnelly, P. Nat Genet 39, 906-13 (2007)) and long range phasing of chip typed Icelandic samples (Kong, A. et al. Nat Genet (2008)).

Moreover, knowledge of the Icelandic genealogy allowed for propagation of genotypic information into individuals for whom neither SNP chip nor sequence data were available, a process referred to as “genealogy-based in silico genotyping”. Reference is made to the combined method of imputing sequence-derived data into phased chromosomes from chip-typed individuals and using genealogy-based in silico genotyping to infer the sequence of un-genotyped individuals as “two-way imputation” (Sulem Pet al Nat Genet. 43(11):1127-30 (2011)). Using this methodology, genotypes for up to about 300,000 individuals may be imputed. The total number of cases entered into this process was 667 individuals with Thyroid cancer.

A two-way imputation-based genome-wide association analysis of the roughly 30 million variants was conducted. The analysis confirmed strong association of marker rs116909374 located on chromosome 14q13.3 with thyroid cancer. The allele specific odds ratio (OR) of allele T of this variant is 1.73, with a P-value of 4.43×10⁻⁰⁷, thus representing a novel risk variant for thyroid cancer. Another marker, rs334725 on chromosome 1p31.3 also showed a significant association with thyroid cancer with the odds ratio of allele G of 1.32, and a P-value of 0.00780769.

Table 6 summarizes the association results for rs113532379 and rs334725 utilizing these further improved techniques. Tables 7 and 8 show results of association of surrogate markers in linkage disequilibrium with rs334725 on chromosome 1 and rs116909374 on chromosome 14, respectively.

TABLE 6
Association results for rs334725-G and rs116909374-T and Thyroid
cancer in Iceland respectively. Results are based on imputations

| | | | | | Ice- | EU- | | | | |
|---|---|---|---|---|---|---|---|---|---|---|
| | | Pos | | | Min All | Min All | | A | A | SEQ |
| Marker | Chr | B 36 | P-Value | OR | Freq % | Freq % | Info | min | maj | ID NO |
| rs334725 | chr1 | 61382637 | 0.00780769 | 1,322 | 6.466 | 3.94 | 0.99298 | G | A | 3 |
| rs116909374 | chr14 | 35808112 | 4.43 × 10⁻⁰⁷ | 1,733 | 4.,879 | 4.46 | 0.98268 | T | C | 43 |

TABLE 7
Association results for markers on Chromosome 1 with Thyroid cancer. Shown
are marker names or ID's (chromosome followed by location in NCBI Build 36),
position in NCBI Build 36, P-values of association with thyroid cancer,
OR for the risk allele, risk allele for the association, i.e. the allele
that is associated with the disease, minor allele frequency, information
content of the imputation, linkage disequilibrium measures r² and D′ to
rs334725, other possible alleles of the marker and reference to Seq
ID No for flanking sequence of the marker.

| | Position | | | Risk | Seq | Minor | | | | | Seq |
|---|---|---|---|---|---|---|---|---|---|---|---|
| | in NCBI | | | (minor) | ID | Allele | | | | Other | ID |
| Marker | B36 | P-value | OR | Allele* | NO | freq | Info | r² | D′ | Allele* | NO |
| chr1: | 61385092 | 0.000843931 | 1.336 | GT | 54 | 9.922 | 0.99031 | 0.624537 | 1 | G | |
| 61385092 | | | | | | | | | | | |
| rs334708 | 61386184 | 0.000869938 | 1.335 | G | 5 | 9.917 | 0.99129 | 0.624066 | 1 | A | 5 |
| chr1: | 61391641 | 0.000952567 | 1.333 | — | | 9.9 | 0.99036 | 0.626257 | 1 | AGCTGTT | 213 |
| 61391641 | | | | AGCCGTT | 55 | | | | | GAT | |
| | | | | GAT | | | | | | | |
| | | | | A | 212 | | | | | | |
| rs334707 | 61388124 | 0.0010176 | 1.331 | C | 6 | 9.838 | 0.9942 | 0.622523 | 1 | T | 6 |
| rs334722 | 61410533 | 0.00267606 | 1.297 | G | 56 | 10.136 | 0.98566 | 0.594558 | 0.981933 | C | 56 |
| rs11207703 | 61401620 | 0.00313733 | 1.207 | C | 57 | 24.815 | 0.97136 | 0.215399 | 0.978551 | T | 57 |
| chr1: | 61399846 | 0.00339401 | 1.322 | AACACAC | 58 | 8.134 | 0.97229 | 0.656011 | 0.934883 | — | |
| 61399846 | | | | ACACAC | | | | | | A | 214 |
| | | | | | | | | | | AACAC | 215 |
| | | | | | | | | | | AACACAC | 216 |
| | | | | | | | | | | AACACAC | 217 |
| | | | | | | | | | | AC | |
| | | | | | | | | | | AACACAC | 218 |
| | | | | | | | | | | ACAC | |
| | | | | | | | | | | AACACAC | 219 |
| | | | | | | | | | | ACACACA | |
| | | | | | | | | | | CACACAC | |
| | | | | | | | | | | AACACAC | 220 |
| | | | | | | | | | | ACACACA | |
| | | | | | | | | | | CACACAC | |
| | | | | | | | | | | AC | |
| chr1: | 61387317 | 0.00344434 | 1.341 | CTTTT | 59 | 7.406 | 0.95582 | 0.895944 | 1 | — | |
| 61387317 | | | | | | | | | | C | 221 |
| | | | | | | | | | | CT | 222 |
| | | | | | | | | | | CTT | 223 |
| chr1: | 61400018 | 0.00355034 | 1.286 | — | | 10.222 | 0.99062 | 0.591644 | 0.981923 | CCCC | 229 |
| 61400018 | | | | CACC | 61 | | | | | | |
| | | | | CACA | 227 | | | | | | |
| | | | | CCC | 228 | | | | | | |
| rs334711 | 61397898 | 0.00361663 | 1.276 | C | 17 | 11.209 | 0.99022 | 0.557406 | 1 | T | 17 |
| rs382704 | 61360454 | 0.00377744 | 1.313 | A | 62 | 8.241 | 0.99958 | 0.71192 | 0.977831 | C | 62 |
| rs4915728 | 61346790 | 0.00378484 | 1.313 | G | 63 | 8.241 | 0.99953 | 0.71192 | 0.977831 | A | 63 |
| rs334732 | 61372987 | 0.0040299 | 1.311 | T | 64 | 8.25 | 0.99856 | 0.71192 | 0.977831 | C | 64 |
| rs334717 | 61411970 | 0.00411297 | 1.282 | C | 65 | 10.215 | 0.9901 | 0.590951 | 0.981917 | T | 65 |
| rs334720 | 61411339 | 0.00417396 | 1.281 | C | 66 | 10.209 | 0.99168 | 0.589914 | 0.981917 | T | 66 |
| chr1: | 61344358 | 0.00618958 | 1.335 | TGC | 67 | 6.386 | 0.98792 | 0.960291 | 0.986898 | — | |
| 61344358 | | | | | | | | | | TGCATCT | 230 |
| | | | | | | | | | | ATCT | |
| | | | | | | | | | | TGCATCT | 231 |
| | | | | | | | | | | TGCATCT | 232 |
| | | | | | | | | | | ATCTATC | |
| | | | | | | | | | | T | |
| | | | | | | | | | | TGCATCT | 233 |
| | | | | | | | | | | ATCTATC | |
| | | | | | | | | | | TATCT | |
| | | | | | | | | | | TGCATCT | 234 |
| | | | | | | | | | | ATCTATC | |
| | | | | | | | | | | TATCTAT | |
| | | | | | | | | | | CT | |
| | | | | | | | | | | TGCATCT | 235 |
| | | | | | | | | | | ATCTATC | |
| | | | | | | | | | | TATCTAT | |
| | | | | | | | | | | CTATCT | |
| | | | | | | | | | | TGCGCAT | 236 |
| | | | | | | | | | | CTATCTA | |
| | | | | | | | | | | TCT | |
| | | | | | | | | | | TGCTATC | 237 |
| | | | | | | | | | | TATCTAT | |
| | | | | | | | | | | CT | |
| | | | | | | | | | | TGCTCTA | 238 |
| | | | | | | | | | | TCTATCT | |
| | | | | | | | | | | ATCTATC | |
| | | | | | | | | | | TATCT | |
| rs334739 | 61364228 | 0.00625221 | 1.335 | G | 68 | 6.333 | 0.9941 | 0.96983 | 0.991231 | A | 68 |
| rs6587912 | 61364965 | 0.00625221 | 1.335 | T | 69 | 6.333 | 0.9941 | 0.96983 | 0.991231 | C | 69 |
| chr1: | 61345726 | 0.00625673 | 1.335 | A | 70 | 6.334 | 0.99412 | 0.96983 | 0.991231 | ACTTTC | 239 |
| 61345726 | | | | | | | | | | | |
| chr1: | 61344341 | 0.00625824 | 1.335 | CCT | 71 | 6.334 | 0.99412 | 0.96983 | 0.991231 | C | 240 |
| 61344341 | | | | | | | | | | | |
| chr1: | 61361965 | 0.00626276 | 1.335 | T | 72 | 6.333 | 0.99409 | 0.96983 | 0.991231 | TG | 241 |
| 61361965 | | | | | | | | | | | |
| rs440611 | 61360268 | 0.00626276 | 1.335 | G | 73 | 6.333 | 0.99409 | 0.96983 | 0.991231 | A | 73 |
| rs2807991 | 61351715 | 0.00626789 | 1.335 | A | 74 | 6.334 | 0.99407 | 0.96983 | 0.991231 | G | 74 |
| chr1: | 61352010 | 0.00627484 | 1.335 | GT | 75 | 6.334 | 0.99407 | 0.96983 | 0.991231 | GTGGAGA | 242 |
| 61352010 | | | | | | | | | | | |
| rs4915586 | 61346745 | 0.00627847 | 1.335 | G | 76 | 6.334 | 0.99412 | 0.96983 | 0.991231 | A | 76 |
| chr1: | 61391641 | 0.006435 | 1.302 | AGCCGTT | 77 | 7.808 | 0.99368 | 0.8184891 | 1 | — | |
| 61391641 | | | | GAT | | | | | | A | 243 |
| | | | | | | | | | | AGCTGTT | 244 |
| | | | | | | | | | | GAT | |
| rs334702 | 61391281 | 0.006435 | 1.302 | T | 10 | 7.808 | 0.99368 | 0.818489 | 1 | C | 10 |
| rs334737 | 61366392 | 0.00647109 | 1.333 | G | 78 | 6.364 | 0.99267 | 0.966653 | 0.991197 | A | 78 |
| rs334729 | 61381061 | 0.00664469 | 1.329 | C | 79 | 6.486 | 0.99299 | 0.991417 | 1 | G | 79 |
| rs334731 | 61374930 | 0.00666749 | 1.332 | A | 80 | 6.342 | 0.99278 | 0.969682 | 0.991197 | G | 80 |
| rs334733 | 61369246 | 0.00669133 | 1.332 | T | 81 | 6.342 | 0.99282 | 0.96983 | 0.991231 | C | 81 |
| rs334734 | 61368886 | 0.00669133 | 1.332 | T | 82 | 6.342 | 0.99282 | 0.96983 | 0.991231 | C | 82 |
| rs395936 | 61377795 | 0.00669133 | 1.332 | C | 83 | 6.342 | 0.99282 | 0.96983 | 0.991231 | T | 83 |
| rs406412 | 61377675 | 0.00669133 | 1.332 | A | 84 | 6.342 | 0.99282 | 0.96983 | 0.991231 | G | 84 |
| rs694151 | 61379533 | 0.00669133 | 1.332 | A | 85 | 6.342 | 0.99282 | 0.96983 | 0.991231 | G | 85 |
| rs694161 | 61379520 | 0.00669133 | 1.332 | A | 86 | 6.342 | 0.99282 | 0.96983 | 0.991231 | C | 86 |
| rs334712 | 61395343 | 0.00669844 | 1.3 | G | 16 | 7.815 | 0.99379 | 0.815512 | 1 | A | 16 |
| rs334730 | 61375294 | 0.0068554 | 1.332 | T | 87 | 6.336 | 0.99179 | 0.969681 | 0.991197 | C | 87 |
| rs4546954 | 61347724 | 0.00724098 | 1.329 | A | 88 | 6.328 | 0.9933 | 0.968883 | 0.991197 | G | 88 |
| rs334726 | 61382117 | 0.00766467 | 1.323 | A | 89 | 6.465 | 0.99284 | 0.995677 | 1 | C | 89 |
| chr1: | 61383184 | 0.00780769 | 1.322 | G | 90 | 6.466 | 0.99298 | 1 | 1 | GAC | 245 |
| 61383184 | | | | | | | | | | | |
| rs334725 | 61382637 | 0.00780769 | 1.322 | G | 3 | 6.466 | 0.99298 | 1 | 1 | A | 3 |
| rs334727 | 61381775 | 0.00790965 | 1.322 | A | 91 | 6.47 | 0.99297 | 1 | 1 | G | 91 |
| rs334728 | 61381595 | 0.00790965 | 1.322 | C | 92 | 6.47 | 0.99297 | 1 | 1 | T | 92 |
| rs12070080 | 61377133 | 0.0080025 | 1.324 | T | 93 | 6.389 | 0.98821 | 0.961045 | 0.982602 | C | 93 |
| rs12064543 | 61377118 | 0.00833212 | 1.322 | G | 94 | 6.387 | 0.99186 | 0.962534 | 0.986899 | A | 94 |
| rs113720032 | 61334598 | 0.0086849 | 1.319 | T | 95 | 6.361 | 0.99596 | 0.967714 | 0.991231 | C | 95 |
| rs17121598 | 61337492 | 0.0086849 | 1.319 | A | 96 | 6.361 | 0.99596 | 0.967714 | 0.991231 | G | 96 |
| rs75541763 | 61338919 | 0.0086849 | 1.319 | T | 97 | 6.361 | 0.99596 | 0.967714 | 0.991231 | C | 97 |
| rs76479717 | 61337200 | 0.0086849 | 1.319 | G | 98 | 6.361 | 0.99596 | 0.967714 | 0.991231 | A | 98 |
| rs77176619 | 61340513 | 0.0086849 | 1.319 | T | 99 | 6.361 | 0.99596 | 0.967714 | 0.991231 | A | 99 |
| rs78217318 | 61337808 | 0.0086849 | 1.319 | G | 100 | 6.361 | 0.99596 | 0.967714 | 0.991231 | T | 100 |
| rs334719 | 61411695 | 0.00902533 | 1.316 | A | 101 | 6.433 | 0.99394 | 0.969905 | 0.986952 | T | 101 |
| rs334723 | 61404617 | 0.0091636 | 1.315 | G | 102 | 6.438 | 0.99238 | 0.969905 | 0.986952 | A | 102 |
| rs334713 | 61394875 | 0.009533 | 1.312 | A | 15 | 6.492 | 0.99379 | 1 | 1 | C | 15 |
| rs334716 | 61412091 | 0.00966983 | 1.313 | G | 103 | 6.369 | 0.99894 | 0.959382 | 0.982668 | A | 103 |
| rs334709 | 61385776 | 0.00985606 | 1.31 | T | 4 | 6.502 | 0.99408 | 1 | 1 | C | 4 |
| rs334710 | 61398460 | 0.00988777 | 1.311 | C | 18 | 6.45 | 0.99363 | 0.965702 | 0.982673 | T | 18 |
| rs334703 | 61390107 | 0.0098974 | 1.31 | C | 9 | 6.502 | 0.99397 | 1 | 1 | G | 9 |
| rs334704 | 61389682 | 0.0098974 | 1.31 | G | 104 | 6.502 | 0.99397 | 1 | 1 | A | 104 |
| rs334705 | 61389660 | 0.0098974 | 1.31 | A | 105 | 6.502 | 0.99397 | 1 | 1 | G | 105 |
| rs334706 | 61388835 | 0.0098974 | 1.31 | G | 7 | 6.502 | 0.99397 | 1 | 1 | C | 7 |
| rs334698 | 61393581 | 0.00997285 | 1.31 | C | 14 | 6.502 | 0.99405 | 1 | 1 | G | 14 |
| rs334699 | 61393084 | 0.00997285 | 1.31 | A | 13 | 6.502 | 0.99405 | 1 | 1 | G | 13 |
| rs334700 | 61392051 | 0.00997285 | 1.31 | A | 12 | 6.502 | 0.99405 | 1 | 1 | G | 12 |
| chr1: | 61409172 | 0.0107077 | 1.309 | TAA | 106 | 6.415 | 0.99392 | 0.978415 | 0.995615 | — | |
| 61409172 | | | | | | | | | | T | 246 |
| | | | | | | | | | | TA | 247 |
| rs334721 | 61411109 | 0.0109294 | 1.311 | A | 107 | 6.271 | 0.9938 | 0.956876 | 0.995524 | C | 107 |
| rs3748543 | 61368577 | 0.0113542 | 1.298 | C | 2 | 6.525 | 0.99301 | 0.96983 | 0.991231 | T | 2 |
| rs77363846 | 61389642 | 0.0124282 | 1.262 | C | 108 | 9.81 | 0.90811 | 0.619369 | 0.949512 | — | |
| | | | | | | | | | | CT | |
| | | | | | | | | | | CTT | 249 |
| chr1: | 61409172 | 0.0142387 | 1.252 | — | | 9.18 | 0.99308 | 0.676876 | 0.98217 | T | 251 |
| 61409172 | | | | TAA | 109 | | | | | | |
| | | | | TA | 250 | | | | | | |
| chr1: | 61410574 | 0.0153916 | 1.289 | TA | 110 | 6.548 | 0.99387 | 0.960561 | 0.982602 | T | 252 |
| 61410574 | | | | | | | | | | | |
| rs334718 | 61411875 | 0.0154981 | 1.288 | G | 111 | 6.55 | 0.99401 | 0.961532 | 0.982668 | C | 111 |
| rs168022 | 61402041 | 0.0187949 | 1.253 | G | 21 | 8.126 | 0.9938 | 0.772974 | 0.982377 | A | 21 |
| rs334724 | 61404590 | 0.0187949 | 1.253 | G | 112 | 8.126 | 0.9938 | 0.772974 | 0.982377 | A | 112 |
| chr1: | 61436916 | 0.0299417 | 1.511 | A | 113 | 1.784 | 0.97679 | 0.253214 | 0.984202 | G | 113 |
| 61436916 | | | | | | | | | | | |
| chr1: | 61393937 | 0.0333671 | 1.257 | CTC | 114 | 7.01 | 0.92372 | 0.776249 | 0.898738 | — | |
| 61393937 | | | | | | | | | | CTA | 253 |
| | | | | | | | | | | CTCAA | 254 |
| | | | | | | | | | | CTCAAA | 255 |
| | | | | | | | | | | CTCAAAA | 256 |
| | | | | | | | | | | CTCAAAA | 257 |
| | | | | | | | | | | A | |
| | | | | | | | | | | CTCAAAA | 258 |
| | | | | | | | | | | AA | |
| rs334697 | 61393935 | 0.0333975 | 1.216 | A | 115 | 9.582 | 0.9592 | 0.669622 | 0.99107 | G | 115 |
| chr1: | 61364696 | 0.0343353 | 1.184 | GAACAC | | 15.976 | 0.859 | 0.328896 | 0.88938 | — | |
| 61364696 | | | | | | | | | | GA | 259 |
| | | | | | | | | | | GAACACA | 260 |
| | | | | | | | | | | C | |
| | | | | | | | | | | GAACACA | 261 |
| | | | | | | | | | | CAC | |
| | | | | | | | | | | GAACACA | 262 |
| | | | | | | | | | | CACAC | |
| | | | | | | | | | | GAACACA | 263 |
| | | | | | | | | | | CACACAC | |
| | | | | | | | | | | GAACACA | 264 |
| | | | | | | | | | | CACACAC | |
| | | | | | | | | | | AC | |
| | | | | | | | | | | GAACACA | 265 |
| | | | | | | | | | | CACACAC | |
| | | | | | | | | | | ACAC | |
| | | | | | | | | | | GAACACA | 266 |
| | | | | | | | | | | CACACAC | |
| | | | | | | | | | | ACACAC | |
| | | | | | | | | | | GAACACA | 267 |
| | | | | | | | | | | CACACAC | |
| | | | | | | | | | | ACACACA | |
| | | | | | | | | | | CAC | |
| | | | | | | | | | | GAACACA | 268 |
| | | | | | | | | | | GAC | |
| | | | | | | | | | | GACACAC | 269 |
| | | | | | | | | | | ACAC | |
| | | | | | | | | | | GAGAACA | 270 |
| | | | | | | | | | | CAC | |
| | | | | | 116 | | | | | GAGAACA | 271 |
| | | | | | | | | | | CACAC | |
| rs146933328 | 61248784 | 0.0397106 | 1.248 | C | 117 | 6.227 | 0.99221 | 0.831689 | 0.936287 | T | 117 |
| rs2807989 | 61350396 | 0.0427751 | 1.217 | T | 118 | 8.459 | 0.9504 | 0.764955 | 0.977905 | A | 118 |
| rs77205085 | 61263854 | 0.043566 | 1.243 | C | 119 | 6.249 | 0.9886 | 0.833316 | 0.936049 | T | 119 |
| rs75521739 | 61322945 | 0.0472728 | 1.275 | G | 120 | 4.731 | 0.99389 | 0.684624 | 0.98793 | A | 120 |
| rs12082005 | 61335642 | 0.0498606 | 1.152 | C | 121 | 16.654 | 0.99591 | 0.319036 | 0.975364 | T | 121 |
| rs334736 | 61366398 | 0.05125 | 1.151 | G | 122 | 16.544 | 0.99851 | 0.31863 | 0.975276 | A | 122 |
| rs75117939 | 61399126 | 0.0519748 | 1.271 | A | 19 | 4.633 | 0.99122 | 0.674337 | 0.99379 | T | 19 |
| chr1: | 61372499 | 0.0520665 | 1.341 | TGTGTGA | 123 | 3.135 | 0.94992 | 0.398277 | 0.877113 | — | |
| 61372499 | | | | GTGTGTG | | | | | | TGTGTGA | 272 |
| | | | | TGTGT | | | | | | TGTGTGA | 273 |
| | | | | | | | | | | GTGT | |
| | | | | | | | | | | TGTGTGA | 274 |
| | | | | | | | | | | GTGTGAG | |
| | | | | | | | | | | TGTGT | |
| | | | | | | | | | | TGTGTGA | 275 |
| | | | | | | | | | | GTGTGT | |
| | | | | | | | | | | TGTGTGA | 276 |
| | | | | | | | | | | GTGTGTG | |
| | | | | | | | | | | AGTGT | |
| | | | | | | | | | | TGTGTGA | 277 |
| | | | | | | | | | | GTGTGTG | |
| | | | | | | | | | | T | |
| | | | | | | | | | | TGTGTGA | 278 |
| | | | | | | | | | | GTGTGTG | |
| | | | | | | | | | | TGT | |
| | | | | | | | | | | TGTGTGT | 279 |
| | | | | | | | | | | GTGTGT | |
| | | | | | | | | | | TGTGTGT | 280 |
| | | | | | | | | | | GTGTGTG | |
| | | | | | | | | | | TGTGT | |
| rs10493302 | 61343980 | 0.0550131 | 1.149 | C | 1 | 16.567 | 0.99745 | 0.318637 | 0.975372 | T | 1 |
| chr1: | 61350384 | 0.0614179 | 1.263 | AT | 124 | 4.6 | 0.9916 | 0.690788 | 0.987997 | A | 281 |
| 61350384 | | | | | | | | | | | |
| rs4430360 | 61371655 | 0.0794298 | 1.134 | A | 125 | 17.065 | 0.99801 | 0.302065 | 0.975034 | T | 125 |
| rs2807990 | 61350879 | 0.0799101 | 1.133 | G | 126 | 17.218 | 0.99883 | 0.302047 | 0.975112 | A | 126 |
| rs334735 | 61366513 | 0.082531 | 1.133 | T | 127 | 17.026 | 0.99699 | 0.302771 | 0.975146 | C | 127 |
| chr1: | 61388105 | 0.0826061 | 1.409 | G | 128 | 1.584 | 0.99586 | 0.236308 | 1 | GT | 282 |
| 61388105 | | | | | | | | | | | |
| rs145491086 | 61379346 | 0.0835309 | 1.407 | T | 129 | 1.582 | 0.99547 | 0.236308 | 1 | G | 129 |
| rs384893 | 61378755 | 0.084921 | 1.132 | A | 130 | 17.092 | 0.99566 | 0.301753 | 0.975126 | G | 130 |
| rs185996257 | 61331346 | 0.0892435 | 1.409 | A | 131 | 1.627 | 0.99517 | 0.255728 | 1 | G | 131 |
| chr1: | 61422633 | 0.0895757 | 1.414 | TA | 132 | 1.601 | 0.97495 | 0.222165 | 0.982096 | T | 283 |
| 61422633 | | | | | | | | | | | |
| chr1: | 61320914 | 0.0911242 | 1.406 | A | 133 | 1.631 | 0.99487 | 0.255728 | 1 | ATT | 284 |
| 61320914 | | | | | | | | | | | |
| rs1391432 | 61331726 | 0.0924569 | 1.128 | G | 134 | 17.161 | 0.99439 | 0.302197 | 0.975114 | A | 134 |
| rs12133298 | 61334728 | 0.0924768 | 1.128 | C | 135 | 17.098 | 0.99677 | 0.302599 | 0.975121 | T | 135 |
| chr1: | 61379679 | 0.0934361 | 1.128 | GG | 136 | 17.188 | 0.99011 | 0.302747 | 0.975134 | GGA | 285 |
| 61379679 | | | | | | | | | | | |
| rs77578111 | 61315160 | 0.0941309 | 1.401 | A | 137 | 1.636 | 0.99691 | 0.255728 | 1 | G | 137 |
| rs149914613 | 61415160 | 0.0999445 | 1.23 | T | 138 | 4.576 | 0.98151 | 0.620379 | 0.96231 | C | 138 |
| rs147893626 | 61205727 | 0.10229 | 1.389 | T | 139 | 1.676 | 0.97404 | 0.245306 | 0.983966 | G | 139 |
| chr1: | 61348369 | 0.102446 | 1.125 | T | 140 | 16.893 | 0.99713 | 0.306396 | 0.975189 | TC | 286 |
| 61348369 | | | | | | | | | | | |
| rs6670604 | 61359656 | 0.107151 | 1.123 | A | 141 | 16.844 | 0.99735 | 0.30654 | 0.975205 | C | 141 |
| rs139873435 | 61234724 | 0.110128 | 1.379 | G | 142 | 1.689 | 0.97348 | 0.245306 | 0.983966 | A | 142 |
| chr1: | 61347753 | 0.11034 | 1.122 | A | 143 | 17.305 | 0.98794 | 0.304175 | 0.975146 | — | |
| 61347753 | | | | | | | | | | AT | 287 |
| | | | | | | | | | | ATT | 288 |
| rs2050544 | 61359826 | 0.11173 | 1.122 | G | 144 | 16.881 | 0.99596 | 0.306568 | 0.975191 | C | 144 |
| rs10889206 | 61331921 | 0.114874 | 1.12 | A | 145 | 16.944 | 0.99585 | 0.306223 | 0.975178 | G | 145 |
| rs1909118 | 61330593 | 0.118895 | 1.119 | A | 146 | 16.935 | 0.99332 | 0.30511 | 0.975088 | G | 146 |
| rs9436630 | 61358261 | 0.124164 | 1.117 | A | 147 | 16.843 | 0.99864 | 0.305497 | 0.975178 | G | 147 |
| chr1: | 61355769 | 0.12435 | 1.117 | G | 148 | 16.964 | 0.99454 | 0.303412 | 0.975137 | GA | 289 |
| 61355769 | | | | | | | | | | | |
| chr1: | 61379676 | 0.162306 | 1.24 | A | 149 | 2.998 | 0.99528 | 0.412574 | 0.980644 | G | 149 |
| 61379676 | | | | | | | | | | | |
| rs115882681 | 61440442 | 0.167386 | 1.17 | A | 37 | 5.869 | 0.98743 | 0.452324 | 0.706568 | G | 37 |
| chr1: | 61347753 | 0.172718 | 1.096 | — | | 21.91 | 0.96733 | 0.223854 | 0.957435 | AT | 291 |
| 61347753 | | | | A | 150 | | | | | | |
| | | | | ATT | 290 | | | | | | |
| rs334738 | 61365343 | 0.233525 | 1.09 | C | 151 | 17.406 | 0.98647 | 0.283731 | 0.949689 | A | 151 |
| chr1: | 61233843 | 0.287129 | 0.875 | T | 152 | 5.53 | 0.98858 | 0.203787 | 0.509873 | — | |
| 61233843 | | | | | | | | | | TAAA | 292 |
| | | | | | | | | | | TA | 293 |
| | | | | | | | | | | TAA | 294 |
| | | | | | | | | | | TAAAA | 295 |
| rs74088754 | 61239451 | 0.290662 | 0.905 | T | 153 | 10.214 | 0.991 | 0.273759 | 0.655209 | C | 153 |
| rs8179472 | 61237902 | 0.301107 | 0.91 | C | 154 | 10.89 | 0.9924 | 0.254646 | 0.663411 | T | 154 |
| rs12026749 | 61237872 | 0.310711 | 0.908 | C | 155 | 10.141 | 0.99008 | 0.280113 | 0.666558 | T | 155 |
| rs58439964 | 61238235 | 0.314706 | 0.91 | G | 156 | 10.231 | 0.99495 | 0.284464 | 0.671042 | C | 156 |
| rs56168787 | 61238550 | 0.317021 | 0.91 | C | 157 | 10.227 | 0.99502 | 0.281582 | 0.666662 | T | 157 |
| rs17121463 | 61241063 | 0.317609 | 0.91 | C | 158 | 10.228 | 0.9947 | 0.281582 | 0.666662 | A | 158 |
| rs74088764 | 61241718 | 0.317609 | 0.91 | A | 159 | 10.228 | 0.9947 | 0.281582 | 0.666662 | T | 159 |
| rs870751 | 61242894 | 0.317609 | 0.91 | T | 160 | 10.228 | 0.9947 | 0.281582 | 0.666662 | G | 160 |
| rs12028122 | 61236014 | 0.317672 | 0.91 | G | 161 | 10.253 | 0.99168 | 0.279795 | 0.665268 | A | 161 |
| rs74088765 | 61242084 | 0.320846 | 0.911 | T | 162 | 10.188 | 0.9967 | 0.282031 | 0.666662 | C | 162 |
| rs75453241 | 61417076 | 0.323824 | 0.913 | A | 163 | 10.557 | 0.98773 | 0.257001 | 0.65141 | G | 163 |
| rs58406226 | 61237093 | 0.325034 | 0.911 | A | 164 | 10.236 | 0.99307 | 0.281582 | 0.666662 | G | 164 |
| rs12024770 | 61236144 | 0.328569 | 0.912 | C | 165 | 10.274 | 0.99087 | 0.27855 | 0.665186 | T | 165 |
| rs12035256 | 61236413 | 0.336972 | 0.914 | T | 166 | 10.319 | 0.99101 | 0.275336 | 0.660639 | C | 166 |
| rs17121462 | 61240190 | 0.33937 | 0.917 | G | 167 | 10.954 | 0.99463 | 0.255973 | 0.663517 | T | 167 |
| rs60032994 | 61240608 | 0.341559 | 0.914 | G | 168 | 10.208 | 0.99469 | 0.281582 | 0.666662 | A | 168 |
| rs58048414 | 61240327 | 0.346301 | 0.915 | G | 169 | 10.196 | 0.9946 | 0.281852 | 0.666744 | A | 169 |
| chr1: | 61243775 | 0.346325 | 0.914 | C | 170 | 9.96 | 0.9935 | 0.284253 | 0.665894 | CT | 296 |
| 61243775 | | | | | | | | | | | |
| rs74088755 | 61239911 | 0.347673 | 0.915 | T | 171 | 10.196 | 0.99502 | 0.281582 | 0.666662 | A | 171 |
| rs74088757 | 61240076 | 0.347673 | 0.915 | C | 172 | 10.196 | 0.99502 | 0.281582 | 0.666662 | G | 172 |
| rs60799423 | 61232276 | 0.365357 | 0.892 | A | 173 | 5.432 | 0.99343 | 0.207673 | 0.521298 | G | 173 |
| rs6699611 | 61229885 | 0.365357 | 0.892 | T | 174 | 5.432 | 0.99343 | 0.207673 | 0.521298 | A | 174 |
| rs72928064 | 61231804 | 0.371648 | 0.894 | A | 175 | 5.422 | 0.99347 | 0.208993 | 0.5244 | G | 175 |
| rs76772552 | 61139231 | 0.481244 | 1.124 | G | 176 | 2.681 | 0.99276 | 0.23283 | 0.756763 | A | 176 |
| rs17121437 | 61221423 | 0.504989 | 1.103 | T | 177 | 3.408 | 0.99664 | 0.347799 | 0.852717 | C | 177 |
| rs10082014 | 61228950 | 0.537283 | 1.096 | C | 178 | 3.405 | 0.997 | 0.343837 | 0.83801 | T | 178 |
| rs77594113 | 61141238 | 0.54709 | 1.105 | G | 179 | 2.737 | 0.99267 | 0.230301 | 0.748996 | T | 179 |
| chr1: | 61422402 | 0.554222 | 1.063 | C | 180 | 8.235 | 0.92247 | 0.225419 | 0.55106 | A | 180 |
| 61422402 | | | | | | | | | | | |
| chr1: | 61364721 | 0.569652 | 1.045 | CA | 181 | 15.531 | 0.94903 | 0.34252 | 0.939852 | — | |
| 61364721 | | | | | | | | | | CAACACA | 297 |
| | | | | | | | | | | CACACAC | |
| | | | | | | | | | | ACT | |
| | | | | | | | | | | CAACACA | 298 |
| | | | | | | | | | | CACACAC | |
| | | | | | | | | | | T | |
| | | | | | | | | | | CACACAC | 299 |
| | | | | | | | | | | ACACACA | |
| | | | | | | | | | | CACA | |
| | | | | | | | | | | CACACAC | 300 |
| | | | | | | | | | | ACACACA | |
| | | | | | | | | | | CACACA | |
| | | | | | | | | | | CACACAC | 301 |
| | | | | | | | | | | ACACACA | |
| | | | | | | | | | | CACACAC | |
| | | | | | | | | | | ACT | |
| | | | | | | | | | | CACACAC | 302 |
| | | | | | | | | | | ACACACA | |
| | | | | | | | | | | CACACAC | |
| | | | | | | | | | | T | |
| | | | | | | | | | | CACACAC | 303 |
| | | | | | | | | | | ACACACA | |
| | | | | | | | | | | CACACT | |
| | | | | | | | | | | CACACAC | 304 |
| | | | | | | | | | | ACACACA | |
| | | | | | | | | | | CACT | |
| | | | | | | | | | | CACACAC | 305 |
| | | | | | | | | | | ACACACA | |
| | | | | | | | | | | CACTCT | |
| | | | | | | | | | | CACACAC | 306 |
| | | | | | | | | | | ACACACA | |
| | | | | | | | | | | CT | |
| | | | | | | | | | | CACACAC | 307 |
| | | | | | | | | | | ACACACA | |
| | | | | | | | | | | CTCT | |
| | | | | | | | | | | CACACAC | 308 |
| | | | | | | | | | | ACACACT | |
| | | | | | | | | | | CACACAC | 309 |
| | | | | | | | | | | ACACACT | |
| | | | | | | | | | | CT | |
| | | | | | | | | | | CACACAC | 310 |
| | | | | | | | | | | ACACT | |
| | | | | | | | | | | CACACAC | 311 |
| | | | | | | | | | | ACACTCT | |
| | | | | | | | | | | CACACAC | 312 |
| | | | | | | | | | | ACT | |
| | | | | | | | | | | CACACAC | 313 |
| | | | | | | | | | | ACTCT | |
| | | | | | | | | | | CACACAC | 314 |
| | | | | | | | | | | GCAAACA | |
| | | | | | | | | | | CACT | |
| | | | | | | | | | | CT | 315 |
| chr1: | 61356450 | 0.578056 | 1.048 | ACA | 182 | 15.202 | 0.83615 | 0.248694 | 0.794659 | — | |
| 61356450 | | | | | | | | | | AA | 316 |
| | | | | | | | | | | AAA | 317 |
| | | | | | | | | | | AC | 318 |
| | | | | | | | | | | ACAA | 319 |
| rs11207707 | 61426431 | 0.60531 | 0.953 | C | 183 | 10.11 | 0.98661 | 0.268919 | 0.654819 | G | 183 |
| chr1: | 61415913 | 0.608848 | 0.949 | T | 184 | 8.144 | 0.98885 | 0.296171 | 0.610426 | — | |
| 61415913 | | | | | | | | | | TTGTGTG | 320 |
| | | | | | | | | | | TTG | 321 |
| | | | | | | | | | | TTGTG | 322 |
| | | | | | | | | | | TTGTGTG | 323 |
| | | | | | | | | | | TG | |
| | | | | | | | | | | TTGTGTG | 324 |
| | | | | | | | | | | TGTG | |
| | | | | | | | | | | TTGTGTG | 325 |
| | | | | | | | | | | TGTGTG | |
| | | | | | | | | | | TTGTGTG | 326 |
| | | | | | | | | | | TGTGTGT | |
| | | | | | | | | | | G | |
| | | | | | | | | | | TTGTGTG | 327 |
| | | | | | | | | | | TGTGTGT | |
| | | | | | | | | | | GTG | |
| | | | | | | | | | | TTGTGTG | 328 |
| | | | | | | | | | | TGTGTGT | |
| | | | | | | | | | | GTGTG | |
| | | | | | | | | | | TTGTGTG | 329 |
| | | | | | | | | | | TGTGTGT | |
| | | | | | | | | | | GTGTGTG | |
| | | | | | | | | | | TTTG | 330 |
| | | | | | | | | | | TTTTGTG | 331 |
| | | | | | | | | | | TGTG | |
| rs12409605 | 61418337 | 0.6392 | 0.954 | C | 185 | 8.624 | 0.9907 | 0.319385 | 0.65822 | T | 185 |
| rs1332781 | 61426026 | 0.6436 | 0.957 | T | 186 | 9.773 | 0.99255 | 0.275639 | 0.653773 | G | 186 |
| rs1779857 | 61236747 | 0.652132 | 0.962 | T | 187 | 12.173 | 0.99118 | 0.218317 | 0.655278 | C | 187 |
| chr1: | 61424548 | 0.667986 | 0.958 | CTCAGTA | 188 | 8.629 | 0.98578 | 0.320874 | 0.65843 | — | |
| 61424548 | | | | TCTCA | | | | | | C | 332 |
| | | | | | | | | | | CTCAGTA | 333 |
| | | | | | | | | | | CTCAGTA | 334 |
| | | | | | | | | | | TC | |
| chr1: | 61424548 | 0.66887 | 0.958 | — | | 8.629 | 0.98577 | 0.320872 | 0.65843 | C | |
| 61424548 | | | | CTCAGTA | 189 | | | | | | |
| | | | | CTCAGTA | 335 | | | | | | |
| | | | | TC | | | | | | | |
| | | | | CTCAGTA | 336 | | | | | | 337 |
| | | | | TCTCA | | | | | | | |
| chr1: | 61423057 | 0.708375 | 0.963 | G | 190 | 8.482 | 0.99544 | 0.3235 | 0.658677 | GA | 338 |
| 61423057 | | | | | | | | | | | |
| rs79484896 | 61423301 | 0.712113 | 0.964 | A | 29 | 8.544 | 0.99208 | 0.325192 | 0.661727 | G | 29 |
| rs12081195 | 61419756 | 0.714224 | 0.964 | A | 26 | 8.539 | 0.99308 | 0.324545 | 0.658677 | G | 26 |
| rs12086591 | 61419744 | 0.714224 | 0.964 | G | 25 | 8.539 | 0.99308 | 0.324545 | 0.658677 | T | 25 |
| rs12091215 | 61419691 | 0.714224 | 0.964 | G | 24 | 8.539 | 0.99308 | 0.324545 | 0.658677 | A | 24 |
| rs55718193 | 61421104 | 0.714224 | 0.964 | G | 28 | 8.539 | 0.99308 | 0.324545 | 0.658677 | A | 28 |
| rs17121794 | 61424408 | 0.715021 | 0.964 | T | 34 | 8.536 | 0.99308 | 0.323032 | 0.658572 | C | 34 |
| rs12065271 | 61423409 | 0.715465 | 0.964 | T | 30 | 8.538 | 0.9928 | 0.324545 | 0.658677 | C | 30 |
| rs79529781 | 61424069 | 0.715465 | 0.964 | G | 31 | 8.538 | 0.9928 | 0.324545 | 0.658677 | A | 31 |
| rs12086218 | 61418240 | 0.743749 | 0.968 | A | 191 | 8.504 | 0.99299 | 0.325635 | 0.658782 | G | 191 |
| rs12086085 | 61417935 | 0.745057 | 0.968 | A | 192 | 8.504 | 0.99281 | 0.326816 | 0.658886 | G | 192 |
| rs75660521 | 61417263 | 0.745057 | 0.968 | T | 193 | 8.504 | 0.99281 | 0.326816 | 0.658886 | C | 193 |
| rs80195615 | 61419091 | 0.778398 | 0.972 | G | 23 | 8.201 | 0.9928 | 0.333142 | 0.659408 | A | 23 |
| rs55916522 | 61421101 | 0.831815 | 0.979 | G | 27 | 8.422 | 0.99302 | 0.326963 | 0.658886 | A | 27 |
| rs914735 | 61419013 | 0.832294 | 0.979 | T | 22 | 8.422 | 0.99294 | 0.326963 | 0.658886 | C | 22 |
| rs1332780 | 61426024 | 0.832614 | 0.979 | T | 35 | 8.422 | 0.99248 | 0.326963 | 0.658886 | C | 35 |
| rs17121791 | 61424221 | 0.833255 | 0.979 | C | 32 | 8.421 | 0.99273 | 0.326963 | 0.658886 | T | 32 |
| rs17121793 | 61424334 | 0.833577 | 0.979 | A | 33 | 8.421 | 0.99274 | 0.326939 | 0.658886 | T | 33 |
| rs11207708 | 61426709 | 0.8339 | 0.979 | G | 36 | 8.42 | 0.99242 | 0.326963 | 0.658886 | A | 36 |
| chr1: | 61422404 | 0.835522 | 0.981 | CCA | 194 | 10.631 | 0.97094 | 0.254127 | 0.650755 | — | |
| 61422404 | | | | | | | | | | CC | 339 |
| | | | | | | | | | | CA | 340 |
| rs12096226 | 61418092 | 0.861681 | 0.983 | G | 195 | 8.38 | 0.99298 | 0.32827 | 0.658991 | A | 195 |
| rs12063945 | 61416830 | 0.86286 | 0.983 | T | 196 | 8.387 | 0.99294 | 0.328746 | 0.658991 | C | 196 |
| chr1: | 61356919 | 0.897853 | 1.017 | AGTGTGT | 198 | 4.908 | 0.94566 | 0.507889 | 0.827172 | — | |
| 61356919 | | | | GTGTGTG | | | | | | AGTGTGT | 343 |
| | | | | T | | | | | | GTGTGTG | |
| | | | | | | | | | | TGTGTGT | |
| | | | | | | | | | | A | 344 |
| | | | | | | | | | | AGT | 345 |
| | | | | | | | | | | AGTGT | 346 |
| | | | | | | | | | | AGTGTGT | 347 |
| | | | | | | | | | | AGTGTGT | 348 |
| | | | | | | | | | | GTGT | |
| | | | | | | | | | | AGTGTGT | 349 |
| | | | | | | | | | | GTGTGT | |
| | | | | | | | | | | AGTGTGT | 350 |
| | | | | | | | | | | GTGTGTG | |
| | | | | | | | | | | TGAGTGT | |
| | | | | | | | | | | AGTGTGT | 351 |
| | | | | | | | | | | GTGTGTG | |
| | | | | | | | | | | TGTGTGA | |
| rs871250 | 61418964 | 0.938336 | 0.993 | C | 199 | 9.953 | 0.99301 | 0.270067 | 0.653125 | T | 199 |
| rs74088771 | 61243825 | 0.992569 | 0.999 | T | 200 | 7.775 | 0.99109 | 0.392917 | 0.675934 | C | 200 |

*The symbol “—” means that the allele can any one of the additional alleles of the marker (when marker contains >2 alleles), excluding the alternate allele.

TABLE 8
Association results for markers on Chromosome 14 with Thyroid cancer. Shown
are marker names or ID's (chromosome followed by location in NCBI Build
36), position in NCBI Build 36, P-values of association with thyroid cancer,
OR for the risk allele, risk allele for the association, i.e. the allele
that is associated with the disease, minor allele frequency, information
content of the imputation, linkage disequilibrium measures r² and D′ to
rs116909374, other possible alleles of the marker and reference to Seq ID
No for flanking sequence of the marker.

| | Position | | | Risk | Seq | Minor | | | | | Seq |
|---|---|---|---|---|---|---|---|---|---|---|---|
| | in NCBI | | | (minor) | ID | allele | | | | Other | ID |
| Marker | B36 | P-value | OR | Allele | NO | freq | Info | r² | D′ | Allele* | NO: |
| rs116909374 | 35808112 | 4.43E−07 | 1.733 | T | 43 | 4.879 | 0.98268 | 1 | 1 | C | 43 |
| chr14: | 35912388 | 9.62E−07 | 1.71 | T | 201 | 4.855 | 0.98276 | 0.989765 | 1 | TA | 352 |
| 35912388 | | | | | | | | | | | |
| rs17175276 | 35847635 | 7.36E−05 | 1.362 | G | 44 | 12.643 | 0.98169 | 0.319561 | 1 | C | 44 |
| rs28690192 | 35850167 | 0.00018559 | 1.34 | A | 202 | 12.615 | 0.98245 | 0.320317 | 1 | C | 202 |
| chr14: | 35971477 | 0.000281322 | 1.874 | T | 49 | 1.774 | 0.98142 | 0.365805 | 1 | C | 49 |
| 35971477 | | | | | | | | | | | |
| chr14:3 | 35867863 | 0.000429579 | 1.314 | TTTAATT | 203 | 13.52 | 0.96162 | 0.280359 | 0.977418 | — | |
| 5867863 | | | | | | | | | | TTTAT | 353 |
| | | | | | | | | | | TATAT | 354 |
| | | | | | | | | | | TTAAT | 355 |
| | | | | | | | | | | TTTATT | 356 |
| | | | | | | | | | | TTTTT | 357 |
| rs118044588 | 35785285 | 0.00122341 | 1.592 | G | 204 | 2.785 | 0.99059 | 0.275415 | 0.65468 | A | 204 |
| chr14: | 35976512 | 0.00176468 | 1.488 | T | 205 | 4.021 | 0.96063 | 0.644705 | 0.89968 | TAAAC | 358 |
| 35976512 | | | | | | | | | | | |
| chr14: | 35591855 | 0.0352864 | 1.632 | ATTGTGT | 206 | 0.994 | 0.98686 | 0.230443 | 1 | — | |
| 35591855 | | | | GTGTGTGT | | | | | | ATTGTGT | 359 |
| | | | | GTG | | | | | | GTGTGTG | |
| | | | | | | | | | | A | 360 |
| | | | | | | | | | | ATGTGTG | 361 |
| | | | | | | | | | | ATGTGTG | 362 |
| | | | | | | | | | | TG | |
| | | | | | | | | | | ATGTGTG | 363 |
| | | | | | | | | | | TGTG | |
| | | | | | | | | | | ATGTGTG | 364 |
| | | | | | | | | | | TGTGTG | |
| | | | | | | | | | | ATGTGTG | 365 |
| | | | | | | | | | | TGTGTGT | |
| | | | | | | | | | | G | |
| | | | | | | | | | | ATTGTGT | 366 |
| | | | | | | | | | | GTG | |
| | | | | | | | | | | ATTGTGT | 367 |
| | | | | | | | | | | GTGTG | |
| | | | | | | | | | | ATTGTGT | 368 |
| | | | | | | | | | | GTGTGTG | |
| | | | | | | | | | | TG | |
| chr14: | 35971015 | 0.0371849 | 1.379 | T | 207 | 2.671 | 0.98691 | 0.231773 | 0.639853 | C | 207 |
| 35971015 | | | | | | | | | | | |
| rs186510185 | 35554277 | 0.0574555 | 1.546 | T | 208 | 1.098 | 0.98305 | 0.206223 | 0.88112 | C | 208 |
| rs118178052 | 35601433 | 0.06815 | 1.565 | A | 209 | 0.927 | 0.98977 | 0.218343 | 1 | G | 209 |
| rs187232017 | 35589152 | 0.0811441 | 1.536 | T | 210 | 0.942 | 0.98213 | 0.216572 | 1 | C | 210 |

*The symbol “—” means that the allele can any one of the additional alleles of the marker (when marker contains >2 alleles), excluding the alternate allele.

1. A method of determining a susceptibility to Thyroid Cancer, the method comprising:
analyzing nucleic acid from a biological sample from a human individual to obtain nucleic acid sequence data for at least one at-risk allele of at least one polymorphic marker selected from the group consisting of rs116909374, rs334725 and rs28933981 and markers in linkage disequilibrium therewith;
wherein different alleles of the at least one polymorphic marker are associated with different susceptibilities to Thyroid Cancer in humans, and
determining a susceptibility to Thyroid Cancer for the human individual from the nucleic acid sequence data.

2-3. (canceled)

4. The method of claim 1, wherein the nucleic acid sequence data is obtained using a method that comprises at least one procedure selected from:
(i) amplification of nucleic acid from the biological sample;
(ii) hybridization assay using a nucleic acid probe and nucleic acid from the biological sample;
(iii) hybridization assay using a nucleic acid probe and nucleic acid obtained by amplification of the biological sample, and
(iv) nucleic acid sequencing.

5-7. (canceled)

8. The method of claim 1, wherein the determining comprises comparing the sequence data to a database containing correlation data between the at least one polymorphic marker and susceptibility to Thyroid Cancer.

9. The method of claim 1, wherein markers in linkage disequilibrium with rs334725 are selected from the group consisting of the markers listed in Table 1.

10. The method of claim 1, wherein markers in linkage disequilbrium with rs334725 are selected from the group consisting of the markers listed in Table 7.

11. The method of claim 1, wherein markers in linkage disequilibrium with rs116909374 are selected from the group consisting of the markers listed in Table 2 and Table 8.

12. (canceled)

13. The method of claim 1, wherein the at least one at-risk allele for thyroid cancer is selected from the risk alleles listed in Table 8 and Table 7.

14. (canceled)

15. The method of claim 1, wherein the at least one at-risk allele is selected from the group consisting of the G allele of rs334725, the T allele of rs116909374 and the T allele of rs28933981.

16-19. (canceled)

20. A method of predicting prognosis of an individual diagnosed with Thyroid Cancer, the method comprising
obtaining nucleic acid sequence data about a human individual about at least one polymorphic marker selected from the group consisting of rs334725, rs116909374, and rs28933981, and markers in linkage disequilibrium therewith, wherein different alleles of the at least one polymorphic marker are associated with different susceptibilities to Thyroid Cancer in humans, and
predicting prognosis of Thyroid Cancer from the nucleic acid sequence data.

21. A method of assessing probability of response of a human individual to a therapeutic agent for preventing, treating and/or ameliorating symptoms associated with Thyroid Cancer, comprising:
obtaining nucleic acid sequence data about a human individual identifying at least one allele of at least one polymorphic marker rs334725, rs116909374, and rs28933981, and markers in linkage disequilibrium therewith, wherein different alleles of the at least one polymorphic marker are associated with different probabilities of response to the therapeutic agent in humans, and
determining the probability of a positive response to the therapeutic agent from the sequence data.

22. A kit for assessing susceptibility to Thyroid Cancer in human individuals, the kit comprising:
reagents for selectively detecting at least one at-risk variant for Thyroid Cancer in the individual, wherein the at least one at-risk variant is selected from the group consisting of rs334725, rs116909374, and rs28933981, and markers in linkage disequilibrium therewith, and
a collection of data comprising correlation data between the at least one at-risk variant and susceptibility to Thyroid Cancer.

23-28. (canceled)

29. An assay for determining a susceptibility to thyroid cancer in a human subject, the assay comprising steps of:
(i) obtaining a nucleic acid sample from a biological sample from the human subject,
(ii) assaying the nucleic acid sample to determine the presence or absence of at least one at-risk allele of at least one polymorphic marker conferring increased susceptibility to thyroid cancer in humans, and
(iii) determining a susceptibility to thyroid cancer for the human subject from the presence or absence of the at least one allele,
wherein the at least one polymorphic marker is selected from the group consisting of rs116909374, rs28933981 and rs334725, and markers in linkage disequilibrium therewith,
wherein determination of the presence of the at least one at-risk allele is indicative of an increased susceptibility to thyroid cancer for the subject.

30-33. (canceled)

34. The assay of claim 29, wherein the at least one at-risk allele is selected from the group consisting of the risk alleles listed in Table 7.

35. The assay of claim 29, wherein the at least one at-risk allele is selected from the group consisting of the risk alleles listed in Table 8.

36-37. (canceled)

38. A system for identifying susceptibility to thyroid cancer in a human subject, the system comprising:
at least one processor;
at least one computer-readable medium;
a susceptibility database operatively coupled to a computer-readable medium of the system and containing population information correlating the presence or absence of at least one marker allele and susceptibility to thyroid cancer in a population of humans;
a measurement tool that receives an input about the human subject and generates information from the input about the presence or absence of the at least one allele in the human subject; and
an analysis tool that:
is operatively coupled to the susceptibility database and the measurement tool,
is stored on a computer-readable medium of the system,
is adapted to be executed on a processor of the system, to compare the information about the human subject with the population information in the susceptibility database and generate a conclusion with respect to susceptibility to thyroid cancer for the human subject;
wherein
the at least one marker allele is an allele of a marker selected from the group consisting of rs116909374, rs334725 and rs28933981, and markers correlated therewith.

39. The system according to claim 38, further including:
a communication tool operatively coupled to the analysis tool, stored on a computer-readable medium of the system and adapted to be executed on a processor of the system to communicate to the subject, or to a medical practitioner for the subject, the conclusion with respect to susceptibility to thyroid cancer for the subject.

40. The system of claim 38, wherein markers correlated with rs116909374 are selected from the group consisting of the markers listed in table 2 and table 8.

41. The system of claim 38, wherein markers correlated with rs334725 are selected from the group consisting of the markers listed in table 1 and table 7.

42. The system of claim 38, wherein the at least one marker allele is selected from the group consisting of the risk alleles listed in Table 7 and Table 8.

43. The system according to claim 38, wherein the measurement tool comprises a tool stored on a computer-readable medium of the system and adapted to be executed by a processor of the system to receive a data input about a subject and determine information about the presence or absence of the at least marker allele in a human subject from the data.

44. The system according to claim 43, wherein the data is genomic sequence information, and the measurement tool comprises a sequence analysis tool stored on a computer readable medium of the system and adapted to be executed by a processor of the system to determine the presence or absence of the at least one marker allele from the genomic sequence information.

45. The system according to claim 44, wherein the input about the human subject is a biological sample from the human subject, and wherein the measurement tool comprises a tool to identify the presence or absence of the at least one marker allele in the biological sample, thereby generating information about the presence or absence of the at least one marker allele in a human subject.

46. The system according to claim 45, wherein the measurement tool includes:
an oligonucleotide microarray containing a plurality of oligonucleotide probes attached to a solid support;
a detector for measuring interaction between nucleic acid obtained from or amplified from the biological sample and one or more oligonucleotides on the oligonucleotide microarray to generate detection data; and
an analysis tool stored on a computer-readable medium of the system and adapted to be executed on a processor of the system, to determine the presence or absence of the at least one marker allele based on the detection data.

47. The system according to claim 38, wherein the measurement tool includes:
a nucleotide sequencer capable of determining nucleotide sequence information from nucleic acid obtained from or amplified from the biological sample; and
an analysis tool stored on a computer-readable medium of the system and adapted to be executed on a processor of the system, to determine the presence or absence of the at least one marker allele based on the nucleotide sequence information.

48. The system according to claim 38, further comprising:
a medical protocol database operatively connected to a computer-readable medium of the system and containing information correlating the presence or absence of the at least one marker allele and medical protocols for human subjects at risk for thyroid cancer; and
a medical protocol routine, operatively connected to the medical protocol database and the analysis routine, stored on a computer-readable medium of the system, and adapted to be executed on a processor of the system, to compare the conclusion from the analysis routine with respect to susceptibility to thyroid cancer for the subject and the medical protocol database, and generate a protocol report with respect to the probability that one or more medical protocols in the database will:
reduce susceptibility to thyroid cancer; or
delay onset of thyroid cancer; or
increase the likelihood of detecting thyroid cancer at an early stage to facilitate early treatment.

49. The system according to claim 39, wherein the communication tool is operatively connected to the analysis routine and comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

50. The system according to claim 49, wherein the communication expresses the susceptibility to thyroid cancer in terms of odds ratio or relative risk or lifetime risk.

51. The system according to claim 49, further comprising:
a medical protocol database operatively connected to a computer-readable medium of the system and containing information correlating the presence or absence of the at least one marker allele and medical protocols for human subjects at risk for thyroid cancer; and
a medical protocol routine, operatively connected to the medical protocol database and the analysis routine, stored on a computer-readable medium of the system, and adapted to be executed on a processor of the system, to compare the conclusion from the analysis routine with respect to susceptibility to thyroid cancer for the subject and the medical protocol database, and generate a protocol report with respect to the probability that one or more medical protocols in the database will:
reduce susceptibility to thyroid cancer; or
delay onset of thyroid cancer; or
increase the likelihood of detecting thyroid cancer at an early stage to facilitate early treatment.
wherein the communication further includes the protocol report.

52. The system according to claim 39, wherein the susceptibility database further includes information about at least one parameter selected from the group consisting of age, sex, ethnicity, race, medical history, weight, diabetes status, blood pressure, family history of thyroid cancer, and smoking history in humans and impact of the at least one parameter on susceptibility to thyroid cancer.

53. A system for assessing or selecting a treatment protocol for a subject diagnosed with thyroid cancer, comprising:
at least one processor;
at least one computer-readable medium;
a medical treatment database operatively connected to a computer-readable medium of the system and containing information correlating the presence or absence of at least one allele of at least one marker selected from the group consisting of rs116909374, rs334725 and rs28933981, and markers correlated therewith, and efficacy of treatment regimens for thyroid cancer;
a measurement tool to receive an input about the human subject and generate information from the input about the presence or absence of the at least one marker allele in a human subject diagnosed with thyroid cancer; and
a medical protocol tool operatively coupled to the medical treatment database and the measurement tool, stored on a computer-readable medium of the system, and adapted to be executed on a processor of the system, to compare the information with respect to presence or absence of the at least one marker allele for the subject and the medical treatment database, and generate a conclusion with respect to at least one of:
the probability that one or more medical treatments will be efficacious for treatment of thyroid cancer for the patient; and
which of two or more medical treatments for thyroid cancer will be more efficacious for the patient.

54. The system according to claim 53, wherein the measurement tool comprises a tool stored on a computer-readable medium of the system and adapted to be executed by a processor of the system to receive a data input about a subject and determine information about the presence or absence of the at least one marker allele in a human subject from the data.

55. The system according to claim 54, wherein the data is genomic sequence information, and the measurement tool comprises a sequence analysis tool stored on a computer readable medium of the system and adapted to be executed by a processor of the system to determine the presence or absence of the at least one marker allele from the genomic sequence information.

56. The system according to claim 55, wherein the input about the human subject is a biological sample from the human subject, and wherein the measurement tool comprises a tool to identify the presence or absence of the at least one marker allele in the biological sample, thereby generating information about the presence or absence of the at least one marker allele in a human subject.

57. The system according to claim 53, further comprising a communication tool operatively connected to the medical protocol routine for communicating the conclusion to the subject, or to a medical practitioner for the subject.

58. The system according to claim 57, wherein the communication tool comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

59. The system according to claim 53, wherein markers correlated with rs116909374 are selected from the group consisting of the markers listed in table 2 and table 8.

60. The system according to claim 53, wherein markers correlated with rs334725 are selected from the group consisting of the markers listed in table 1 and table 7.

61. The system according to claim 53, wherein the at least one marker allele is selected from the group consisting of the risk alleles listed in Table 7 and Table 8.

62. (canceled)

63. The method according to claim 1, wherein linkage disequilibrium between markers is characterized by values of r² of at least 0.2.

64. The method according to claim 1, wherein linkage disequilibrium between markers is characterized by values of r² of at least 0.5.
